SEQUENCE LISTING (5 sequences)

SEQ ID NO: 1 - protein, 16 aa, Homo sapiens
KTSLHRLKPDTVPAPC

SEQ ID NO: 2 - DNA, 927 bp, Homo sapiens
atgcccgggcaagaactcaggacggtgaatggctctcagatgctcctggtgttgctggtgctctcgtggctgccgcatgggggcgccctgtctctggccgaggcgagccgcgcaagtttcccgggaccctcagagttgcactccgaagactccagattccgagagttgcggaaacgctacgaggacctgctaaccaggctgcgggccaaccagagctgggaagattcgaacaccgacctcgtcccggcccctgcagtccggatactcacgccagaagtgcggctgggatccggcggccacctgcacctgcgtatctctcgggccgcccttcccgaggggctccccgaggcctcccgccttcaccgggctctgttccggctgtccccgacggcgtcaaggtcgtgggacgtgacacgaccgctgcggcgtcagctcagccttgcaagaccccaggcgcccgcgctgcacctgcgactgtcgccgccgccgtcgcagtcggaccaactgctggcagaatcttcgtccgcacggccccagctggagttgcacttgcggccgcaagccgccagggggcgccgcagagcgcgtgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcatatga

SEQ ID NO: 3 - DNA, 5912 bp, Homo sapiens
atgcagcccccttcactgctgctgctgctgctgctgctgctgctgctatgtgtctcagtggtcagacccagagggctgctgtgtgggagtttcccagaaccctgtgccaatggaggcacctgcctgagcctgtctctgggacaagggacctgccagtgtgcccctggcttcctgggtgagacgtgccagtttcctgacccctgccagaacgcccagctctgccaaaatggaggcagctgccaagccctgcttcccgctcccctagggctccccagctctccctctccattgacacccagcttcttgtgcacttgcctccctggcttcactggtgagagatgccaggccaagcttgaagacccttgtcctccctccttctgttccaaaaggggccgctgccacatccaggcctcgggccgcccacagtgctcctgcatgcctggatggacaggtgagcagtgccagcttcgggacttctgttcagccaacccatgtgttaatggaggggtgtgtctggccacatacccccagatccagtgccactgcccaccgggcttcgagggccatgcctgtgaacgtgatgtcaacgagtgcttccaggacccaggaccctgccccaaaggcacctcctgccataacaccctgggctccttccagtgcctctgccctgtggggcaggagggtccacgttgtgagctgcgggcaggaccctgccctcctaggggctgttcgaatgggggcacctgccagctgatgccagagaaagactccacctttcacctctgcctctgtcccccaggtttcataggcccagactgtgaggtgaatccagacaactgtgtcagccaccagtgtcagaatgggggcacttgccaggatgggctggacacctacacctgcctctgcccagaaacctggacaggctgggactgctccgaagatgtggatgagtgtgagacccagggtccccctcactgcagaaacgggggcacctgccagaactctgctggtagctttcactgcgtgtgtgtgagtggctggggcggcacaagctgtgaggagaacctggatgactgtattgctgccacctgtgccccgggatccacctgcattgaccgggtgggctctttctcctgcctctgcccacctggacgcacaggactcctgtgccacttggaagacatgtgtctgagccagccgtgccatggggatgcccaatgcagcaccaaccccctcacaggctccacactctgcctgtgtcagcctggctattcggggcccacctgccaccaggacctggacgagtgtctgatggcccagcaaggcccaagtccctgtgaacatggcggttcctgcctcaacactcctggctccttcaactgcctctgtccacctggctacacaggctcccgttgtgaggctgatcacaatgagtgcctctcccagccctgccacccaggaagcacctgtctggacctacttgccaccttccactgcctctgcccgccaggcttagaagggcagctctgtgaggtggagaccaacgagtgtgcctcagctccctgcctgaaccacgcggattgccatgacctgctcaacggcttccagtgcatctgcctgcctggattctccggcacccgatgtgaggaggatatcgatgagtgcagaagctctccctgtgccaatggtgggcagtgccaggaccagcctggagccttccactgcaagtgtctcccaggctttgaagggccacgctgtcaaacagaggtggatgagtgcctgagtgacccatgtcccgttggagccagctgccttgatcttccaggagccttcttttgcctctgcccctctggtttcacaggccagctctgtgaggttcccctgtgtgctcccaacctgtgccagcccaagcagatatgtaaggaccagaaagacaaggccaactgcctctgtcctgatggaagccctggctgtgccccacctgaggacaactgcacctgccaccacgggcactgccagagatcctcatgtgtgtgtgacgtgggttggacggggccagagtgtgaggcagagctagggggctgcatctctgcaccctgtgcccatggggggacctgctacccccagccctctggctacaactgcacctgccctacaggctacacaggacccacctgtagtgaggagatgacagcttgtcactcagggccatgtctcaatggcggctcctgcaaccctagccctggaggctactactgcacctgccctccaagccacacagggccccagtgccaaaccagcactgactactgtgtgtctgccccgtgcttcaatgggggtacctgtgtgaacaggcctggcaccttctcctgcctctgtgccatgggcttccagggcccgcgctgtgagggaaagctccgccccagctgtgcagacagcccctgtaggaatagggcaacctgccaggacagccctcagggtccccgctgcctctgccccactggctacaccggaggcagctgccagactctgatggacttatgtgcccagaagccctgcccacgcaattcccactgcctccagactgggccctccttccactgcttgtgcctccagggatggaccgggcctctctgcaaccttccactgtcctcctgccagaaggctgcactgagccaaggcatagacgtctcttccctttgccacaatggaggcctctgtgtcgacagcggcccctcctatttctgccactgcccccctggattccaaggcagcctgtgccaggatcacgtgaacccatgtgagtccaggccttgccagaacggggccacctgcatggcccagcccagtgggtatctctgccagtgtgccccaggctacgatggacagaactgctcaaaggaactcgatgcttgtcagtcccaaccctgtcacaaccatggaacctgtactcccaaacctggaggattccactgtgcctgccctccaggctttgtggggctacgctgtgagggagacgtggacgagtgtctggaccagccctgccaccccacaggcactgcagcctgccactctctggccaatgccttctactgccagtgtctgcctggacacacaggccagtggtgtgaggtggagatagacccctgccacagccaaccctgctttcatggagggacctgtgaggccacagcaggatcacccctgggtttcatctgccactgccccaagggttttgaaggccccacctgcagccacagggccccttcctgcggcttccatcactgccaccacggaggcctgtgtctgccctcccctaagccaggcttcccaccacgctgtgcctgcctcagtggctatgggggtcctgactgcctgaccccaccagctcctaaaggctgtggccctccctccccatgcctatacaatggcagctgctcagagaccacgggcttggggggcccaggctttcgatgctcctgccctcacagctctccagggccccggtgtcagaaacccggagccaaggggtgtgagggcagaagtggagatggggcctgcgatgctggctgcagtggcccgggaggaaactgggatggaggggactgctctctgggagtcccagacccctggaagggctgcccctcccactctcggtgctggcttctcttccgggacgggcagtgccacccacagtgtgactctgaagagtgtctgtttgatggctacgactgtgagacccctccagcctgcactccagcctatgaccagtactgccatgatcacttccacaacgggcactgtgagaaaggctgcaacactgcagagtgtggctgggatggaggtgactgcaggcctgaagatggggacccagagtgggggccctccctggccctgctggtggtactgagccccccagccctagaccagcagctgtttgccctggcccgggtgctgtccctgactctgagggtaggactctgggtaaggaaggatcgtgatggcagggacatggtgtacccctatcctggggcccgggctgaagaaaagctaggaggaactcgggaccccacctatcaggagagagcagcccctcaaacgcagcccctgggcaaggagaccgactccctcagtgctgggtttgtggtggtcatgggtgtggatttgtcccgctgtggccctgaccacccggcatcccgctgtccctgggaccctgggcttctactccgcttccttgctgcgatggctgcagtgggagccctggagcccctgctgcctggaccactgctggctgtccaccctcatgcagggaccgcaccccctgccaaccagcttccctggcctgtgctgtgctccccagtggccggggtgattctcctggccctaggggctcttctcgtcctccagctcatccggcgtcgacgccgagagcatggagctctctggctgccccctggtttcactcgacggcctcggactcagtcagctccccaccgacgccggcccccactaggcgaggacagcattggtctcaaggcactgaagccaaaggcagaagttgatgaggatggagttgtgatgtgctcaggccctgaggagggagaggaggtgggccaggctgaagaaacaggcccaccctccacgtgccagctctggtctctgagtggtggctgtggggcgctccctcaggcagccatgctaactcctccccaggaatctgagatggaagcccctgacctggacacccgtggacctgatggggtgacacccctgatgtcagcagtttgctgtggggaagtacagtccgggaccttccaaggggcatggttgggatgtcctgagccctgggaacctctgctggatggaggggcctgtccccaggctcacaccgtgggcactggggagacccccctgcacctggctgcccgattctcccggccaaccgctgcccgccgcctccttgaggctggagccaaccccaaccagccagaccgggcagggcgcacaccccttcatgctgctgtggctgctgatgctcgggaggtctgccagcttctgctccgtagcagacaaactgcagtggacgctcgcacagaggacgggaccacacccttgatgctggctgccaggctggcggtggaagacctggttgaagaactgattgcagcccaagcagacgtgggggccagagataaatgggggaaaactgcgctgcactgggctgctgccgtgaacaacgcccgagccgcccgctcgcttctccaggccggagccgataaagatgcccaggacaacagggagcagacgccgctattcctggcggcgcgggaaggagcggtggaagtagcccagctactgctggggctgggggcagcccgagagctgcgggaccaggctgggctagcgccggcggacgtcgctcaccaacgtaaccactgggatctgctgacgctgctggaaggggctgggccaccagaggcccgtcacaaagccacgccgggccgcgaggctgggcccttcccgcgcgcacggacggtgtcagtaagcgtgcccccgcatgggggcggggctctgccgcgctgccggacgctgtcagccggagcaggccctcgtgggggcggagcttgtctgcaggctcggacttggtccgtagacttggctgcgcgggggggcggggcctattctcattgccggagcctctcgggagtaggagcaggaggaggcccgacccctcgcggccgtaggttttctgcaggcatgcgcgggcctcggcccaaccctgcgataatgcgaggaagatacggagtggctgccgggcgcggaggcagggtctcaacggatgactggccctgtgattgggtggccctgggagcttgcggttctgcctccaacattccgatcccgcctccttg

SEQ ID NO: 4 - protein, 2003 aa, Homo sapiens
MQPPSLLLLLLLLLLLCVSVVRPRGLLCGSFPEPCANGGTCLSLSLGQGTCQCAPGFLGETCQFPDPCQNAQLCQNGGSCQALLPAPLGLPSSPSPLTPSFLCTCLPGFTGERCQAKLEDPCPPSFCSKRGRCHIQASGRPQCSCMPGWTGEQCQLRDFCSANPCVNGGVCLATYPQIQCHCPPGFEGHACERDVNECFQDPGPCPKGTSCHNTLGSFQCLCPVGQEGPRCELRAGPCPPRGCSNGGTCQLMPEKDSTFHLCLCPPGFIGPDCEVNPDNCVSHQCQNGGTCQDGLDTYTCLCPETWTGWDCSEDVDECETQGPPHCRNGGTCQNSAGSFHCVCVSGWGGTSCEENLDDCIAATCAPGSTCIDRVGSFSCLCPPGRTGLLCHLEDMCLSQPCHGDAQCSTNPLTGSTLCLCQPGYSGPTCHQDLDECLMAQQGPSPCEHGGSCLNTPGSFNCLCPPGYTGSRCEADHNECLSQPCHPGSTCLDLLATFHCLCPPGLEGQLCEVETNECASAPCLNHADCHDLLNGFQCICLPGFSGTRCEEDIDECRSSPCANGGQCQDQPGAFHCKCLPGFEGPRCQTEVDECLSDPCPVGASCLDLPGAFFCLCPSGFTGQLCEVPLCAPNLCQPKQICKDQKDKANCLCPDGSPGCAPPEDNCTCHHGHCQRSSCVCDVGWTGPECEAELGGCISAPCAHGGTCYPQPSGYNCTCPTGYTGPTCSEEMTACHSGPCLNGGSCNPSPGGYYCTCPPSHTGPQCQTSTDYCVSAPCFNGGTCVNRPGTFSCLCAMGFQGPRCEGKLRPSCADSPCRNRATCQDSPQGPRCLCPTGYTGGSCQTLMDLCAQKPCPRNSHCLQTGPSFHCLCLQGWTGPLCNLPLSSCQKAALSQGIDVSSLCHNGGLCVDSGPSYFCHCPPGFQGSLCQDHVNPCESRPCQNGATCMAQPSGYLCQCAPGYDGQNCSKELDACQSQPCHNHGTCTPKPGGFHCACPPGFVGLRCEGDVDECLDQPCHPTGTAACHSLANAFYCQCLPGHTGQWCEVEIDPCHSQPCFHGGTCEATAGSPLGFICHCPKGFEGPTCSHRAPSCGFHHCHHGGLCLPSPKPGFPPRCACLSGYGGPDCLTPPAPKGCGPPSPCLYNGSCSETTGLGGPGFRCSCPHSSPGPRCQKPGAKGCEGRSGDGACDAGCSGPGGNWDGGDCSLGVPDPWKGCPSHSRCWLLFRDGQCHPQCDSEECLFDGYDCETPPACTPAYDQYCHDHFHNGHCEKGCNTAECGWDGGDCRPEDGDPEWGPSLALLVVLSPPALDQQLFALARVLSLTLRVGLWVRKDRDGRDMVYPYPGARAEEKLGGTRDPTYQERAAPQTQPLGKETDSLSAGFVVVMGVDLSRCGPDHPASRCPWDPGLLLRFLAAMAAVGALEPLLPGPLLAVHPHAGTAPPANQLPWPVLCSPVAGVILLALGALLVLQLIRRRRREHGALWLPPGFTRRPRTQSAPHRRRPPLGEDSIGLKALKPKAEVDEDGVVMCSGPEEGEEVGQAEETGPPSTCQLWSLSGGCGALPQAAMLTPPQESEMEAPDLDTRGPDGVTPLMSAVCCGEVQSGTFQGAWLGCPEPWEPLLDGGACPQAHTVGTGETPLHLAARFSRPTAARRLLEAGANPNQPDRAGRTPLHAAVAADAREVCQLLLRSRQTAVDARTEDGTTPLMLAARLAVEDLVEELIAAQADVGARDKWGKTALHWAAAVNNARAARSLLQAGADKDAQDNREQTPLFLAAREGAVEVAQLLLGLGAARELRDQAGLAPADVAHQRNHWDLLTLLEGAGPPEARHKATPGREAGPFPRARTVSVSVPPHGGGALPRCRTLSAGAGPRGGGACLQARTWSVDLAARGGGAYSHCRSLSGVGAGGGPTPRGRRFSAGMRGPRPNPAIMRGRYGVAAGRGGRVSTDDWPCDWVALGACGSASNIPIPPPCLTPSPERGSPQLDCGPPALQEMPINQGGEGKK

SEQ ID NO: 5 - protein, 308 aa, Homo sapiens
MPGQELRTVNGSQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

(19) United States
(12) Patent Application Publication
(10) Pub. No.: US 2023/0212274 A1
(43) Pub. Date: Jul. 6, 2023

(54) METHOD FOR TREATING ASTHMA OR ALLERGIC DISEASE

(71) Applicant: THE CHILDREN'S MEDICAL CENTER CORPORATION, Boston, MA (US)

(72) Inventors: Talal Amine Chatila, Belmont, MA (US); Hani Harb, Brookline, MA (US)

(73) Assignee: THE CHILDREN'S MEDICAL CENTER CORPORATION, Boston, MA (US)

(21) Appl. No.: 17/800,837

(22) PCT Filed: Feb. 16, 2021
(86) PCT No.: PCT/US2021/018174

§ 371 Date: Aug. 18, 2022

Related U.S. Application Data
(60) Provisional application No. 62/979,602, filed on Feb. 21, 2020.

(51) Int. Cl. C07K 16/22; C07K 16/32; G01N 33/68; A61P 11/00

(52) U.S. Cl. CPC C07K 16/22; A61P 11/00; C07K 16/32; G01N 33/6893; C07K 2317/24

(57) ABSTRACT

Described herein are methods and compositions for treating asthma or an allergic disease. Aspects of the invention relate to administering to a subject an agent that targets the Wnt or Hippo Signaling pathway, or Growth-differentiation factor 15 (GDF15), either alone or in combination. In certain embodiments, the subject is further administered a Notch4 inhibitor.

### CROSS-REFERENCE PARAGRAPH

This application is a 35 U.S.C. §371 National Phase Entry Application of International Patent Application No. PCT/US21/18174 filed Feb. 16, 2021, which designated the U.S., which claims benefit under 35 U.S.C. §119(e) of U.S. Provisional Application 62/979,602 filed on Feb. 21, 2020, the contents of which are incorporated herein in their entireties by reference.

### GOVERNMENT SUPPORT

This invention was made with Government support under Grant Nos R01 AI115699 and R01AI065617 awarded by the National Institutes of Health. The Government has certain rights in the invention.

### SEQUENCE LISTING

The instant application contains a Sequence Listing which has been submitted electronically in ASCII format and is hereby incorporated by reference in its entirety. Said ASCII copy, created on Mar. 9, 2021, is named 701039-096930WOPT_SL.txt and is 29,826 bytes in size.

### BACKGROUND

Exposure to traffic-related particulate matter (PM) promotes asthma and allergic diseases. However, the precise cellular and molecular mechanisms by which PM exposure acts to mediate these effects remain unclear. An understanding of cellular targets and signaling pathways critical for the augmentation of allergic airway inflammation induced by ambient ultra-fine particles (UFP) is essential for developing therapeutics to treat or prevent asthma and allergic diseases.

### SUMMARY

Notch4 expression on regulatory T cells has been identified by the inventors to be critical for immune tolerance breakdown in Asthma, leading to tissue inflammation and disease exacerbation and persistence. The invention described herein is related, in part, to the discovery of the mechanism downstream of Notch4 that promotes tissue inflammation in asthma, involving the activation by Notch4 of the Wingless signaling pathway (Wnt) and its main effector β-catenin, leading to the expression of the cytokine growth and differentiation factor 15 (GDF15). Data presented herein in the Examples show that this cytokine activates lung innate lymphoid cells type 2 (ILC2) to produce the pro-asthmatic cytokine IL-13. Inhibition of GDF15 inhibited lung inflammation, providing a novel target for therapy for asthma and related inflammatory lung disorders. Accordingly, one aspect described herein provides a method for treating asthma or an allergic disease, comprising administering to a subject having asthma or an allergic disease an effective amount of an agent that inhibits Wnt signaling.

Another aspect described herein provides a method for treating asthma or an allergic disease, comprising administering to a subject having asthma or an allergic disease an effective amount of an agent that inhibits Hippo signaling.

Yet another aspect described herein provides a method for treating asthma or an allergic disease, comprising administering to a subject having asthma or an allergic disease an effective amount of an inhibitor of Growth-differentiation factor 15 (GDF15).

In one embodiment of any aspect provided herein, the method further comprises administering an agent that inhibits Growth/differentiation factor 15 (GDF15).

In one embodiment of any aspect provided herein, the method further comprises administering an agent that inhibits Notch4.

In one embodiment of any aspect provided herein, the method further comprises administering an agent that inhibits Wnt signaling and an agent that inhibits Hippo signaling.

In one embodiment of any aspect provided herein, the method further comprises, prior to administering, diagnosing a subject as having asthma or an allergic disease.

In one embodiment of any aspect provided herein, the method further comprises, prior to administering, receiving the results of an assay that diagnoses a subject as having asthma or an allergic disease.

In one embodiment of any aspect provided herein, the asthma is selected from the list consisting of allergic asthma, asthma without allergies, aspirin exacerbated respiratory disease, exercise induced asthma, cough variant, and occupational asthma.

In one embodiment of any aspect provided herein, the allergic disease is selected from the list consisting of allergic rhinitis, sinusitis, otitis media, atopic dermatitis, urticaria, angioedema, and anaphylaxis.

In one embodiment of any aspect provided herein, the agent is selected from the group consisting of a small molecule, an antibody, a peptide, a genome editing system, an antisense oligonucleotide, and an RNAi.

In one embodiment of any aspect provided herein, the antibody is a humanized antibody.

In one embodiment of any aspect provided herein, the RNAi is a microRNA, an siRNA, or a shRNA.

In one embodiment of any aspect provided herein, the small molecule is an inhibitor of Wnt signaling, and is selected from the group consisting of XAV-939, ICG-001, IWR-1-endo, Wnt-C59 (C59), LGK-974, JW55, ETC-159, iCRT14, KY02111, IWP-2, IWP-L6, Isoquercitrin, PNU-74654, CP21R7 (CP21), Salinomycin (from Streptomyces albus), Adavivint (SM04690), FH535, IWP-O1, LF3, WIKI4, Triptonide, PRI-724, GNF-6231, KYA1797K, Methyl Vanillate, iCRT3, WAY-316606, and SKL2001.

In one embodiment of any aspect provided herein, the small molecule is an inhibitor of Hippo signaling, and is selected from the group consisting of (R)-PFI 2 hydrochloride, Verteporfin, YAP inhibitor, XMU MP 1, Ki 16425, and Ro 08-2750.

In one embodiment of any aspect provided herein, the peptide is an inhibitor of GDF15, and has a sequence of SEQ ID NO: 1.

In one embodiment of any aspect provided herein, the antibody is an anti-Notch4 antibody.

In one embodiment of any aspect provided herein, inhibiting GDF15 is inhibiting GFD15 expression level or activity. For example, GFD15 expression level or activity is reduced by at least 50%, 60%, 70%, 80%, 90%, 95%, or more are compared to an appropriate control.

In one embodiment of any aspect provided herein, inhibiting Wnt signaling reduces the population of Th2 effector cells.

In one embodiment of any aspect provided herein, inhibiting Hippo signaling reduces the population of Th17 effector cells

In one embodiment of any aspect provided herein, inhibiting GDF15 reduces the population of group 2 innate lymphoid cell (ILC2).

In one embodiment of any aspect provided herein, the population is reduced at least 50%, 60%, 70%, 80%, 90%, 95%, or more are compared to an appropriate control.

In one embodiment of any aspect provided herein, the method further comprises administering at least one additional anti-asthma therapeutic.

In one embodiment of any aspect provided herein, the method further comprises administering at least one additional anti-allergic disease therapeutic.

One aspect described herein provides a method for preventing asthma or an allergic disease, comprising administering to a subject at risk of having asthma or an allergic disease an effective amount of an agent that inhibits Wnt signaling.

One aspect described herein provides a method for preventing asthma or an allergic disease, comprising administering to a subject at risk of having asthma or an allergic disease an effective amount of an agent that inhibits Hippo signaling.

One aspect described herein provides a method for preventing asthma or an allergic disease, comprising administering to a subject at risk of having asthma or an allergic disease an effective amount of an inhibitor of Growth-differentiation factor 15 (GDF15).

In one embodiment of any aspect provided herein, the method further comprises, prior to administering, diagnosing a subject as being at risk of having asthma or an allergic disease.

In one embodiment of any aspect provided herein, the method further comprises, prior to administering, receiving the results of an assay that diagnoses a subject as being at risk of having asthma or an allergic disease.

One aspect described herein provides a composition for preventing or treating asthma or an allergic disease, comprising an agent that inhibits Wnt signaling and a pharmaceutically acceptable carrier.

One aspect described herein provides a composition for preventing or treating asthma or an allergic disease, comprising an agent that inhibits Hippo signaling and a pharmaceutically acceptable carrier.

One aspect described herein provides a composition for preventing or treating asthma or an allergic disease, comprising an agent that inhibits GDF15 and a pharmaceutically acceptable carrier.

In one embodiment of any aspect provided herein, the compositions further comprise an agent that inhibits Growth/differentiation factor 15 (GDF15).

In one embodiment of any aspect provided herein, the compositions further comprise an agent that inhibits Notch4.

In one embodiment of any aspect provided herein, the compositions further comprise an agent that inhibits Wnt signaling and an agent that inhibits Hippo signaling.

One aspect described herein provides an agent that inhibits the Wnt signaling pathway.

One aspect described herein provides an agent that inhibits the Hippo signaling pathway.

One aspect described herein provides an agent that inhibits GDF15.

One aspect described herein provides an agent that inhibits Notch4.

One aspect described herein provides a method for treating asthma or an allergic disease, the method comprising: (a) obtaining a biological sample from a subject; (b) measuring the level of Notch4 in the biological sample of (a); (c) comparing the level of (b) with a reference level, wherein a subject is identified as having asthma or an allergic disease if the level of (b) is greater than a reference level; and (d) administering to the subject identified as having at risk asthma or an allergic disease any of the compositions or agents described herein.

One aspect described herein provides a method for preventing asthma or an allergic disease, the method comprising: (a) obtaining a biological sample from a subject; (b) measuring the level of Notch4 in the biological sample of (a); (c) comparing the level of (b) with a reference level, wherein a subject is identified as being at risk of having asthma or an allergic disease if the level of (b) is greater than a reference level; and (d) administering to the subject identified as having at risk asthma or an allergic disease any of the compositions or agents described herein.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with asthma or an allergic disease. The term “treating” includes reducing or alleviating at least one adverse effect or symptom of an asthma or an allergic disease (e.g., inflamed airway). Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein “preventing” or “prevention” refers to any methodology where the disease state or disorder (e.g., asthma or an allergic disease) does not occur due to the actions of the methodology (such as, for example, administration of an agent that inhibits Wnt or Hippo signaling pathways, or GDF15, or a composition thereof described herein). In one aspect, it is understood that prevention can also mean that the disease is not established to the extent that occurs in untreated controls. For example, there can be a 5, 10, 15, 20, 25, 30, 35, 40, 50, 60, 70, 80, 90, or 100% reduction in the establishment of disease or disorder frequency relative to untreated controls. Accordingly, prevention of a disease or disorder encompasses a reduction in the likelihood that a subject will develop the disease, relative to an untreated subject (e.g. a subject who is not treated with a composition comprising a microbial consortium as described herein).

As used herein, the term “administering,” refers to the placement of a therapeutic (e.g., agent that inhibits Wnt or Hippo signaling pathways, or GDF15, or a composition thereof described herein) or pharmaceutical composition as disclosed herein into a subject by a method or route which results in at least partial delivery of the agent to the subject. Pharmaceutical compositions comprising agents as disclosed herein can be administered by any appropriate route which results in an effective treatment in the subject, e.g., via direction administration to the lung, such as inhalation.

As used herein, a “subject” means a human or animal. Usually the animal is a vertebrate such as a primate, rodent, domestic animal or game animal. Primates include, for example, chimpanzees, cynomolgus monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of disease e.g., asthma or an allergic disease. A subject can be male or female. A subject can be an infant (e.g., less than one year of age), a child (e.g., greater than one year, but less than 18 years of age), or an adult (e.g., greater than 18 years of age).

A subject can be one who has been previously diagnosed with or identified as suffering from or having a disease or disorder in need of treatment (e.g., asthma or an allergic disease) or one or more complications related to such a disease or disorder, and optionally, have already undergone treatment for the disease or disorder or the one or more complications related to the disease or disorder. Alternatively, a subject can also be one who has not been previously diagnosed as having such disease or disorder (e.g., asthma or an allergic disease) or related complications. For example, a subject can be one who exhibits one or more risk factors for the disease or disorder or one or more complications related to the disease or disorder or a subject who does not exhibit risk factors.

As used herein, an “agent” refers to e.g., a molecule, protein, peptide, antibody, or nucleic acid, that inhibits expression of a polypeptide or polynucleotide, or binds to, partially or totally blocks stimulation, decreases, prevents, delays activation, inactivates, desensitizes, or down regulates the activity of the polypeptide or the polynucleotide. Agents that inhibit, for example, Wnt of Hippo signaling pathways or GDF15, e.g., inhibit expression, e.g., translation, post-translational processing, stability, degradation, or nuclear or cytoplasmic localization of a polypeptide, or transcription, post transcriptional processing, stability or degradation of a polynucleotide or bind to, partially or totally block stimulation, DNA binding, transcription factor activity or enzymatic activity, decrease, prevent, delay activation, inactivate, desensitize, or down regulate the activity of a polypeptide or polynucleotide. An agent can act directly or indirectly.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

The agent can be a molecule from one or more chemical classes, e.g., organic molecules, which may include organometallic molecules, inorganic molecules, genetic sequences, etc. Agents may also be fusion proteins from one or more proteins, chimeric proteins (for example domain switching or homologous recombination of functionally significant regions of related or different molecules), synthetic proteins or other protein variations including substitutions, deletions, insertion and other variants.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “RNAi” as used herein refers to interfering RNA or RNA interference. RNAi refers to a means of selective post-transcriptional gene silencing by destruction of specific mRNA by molecules that bind and inhibit the processing of mRNA, for example inhibit mRNA translation or result in mRNA degradation. As used herein, the term “RNAi” refers to any type of interfering RNA, including but are not limited to, siRNA, shRNA, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein).

The term “decrease”, “reduced”, “reduction”, or “inhibit” are all used herein to mean a decrease by a statistically significant amount. In some embodiments, “decrease”, “reduced”, “reduction”, or “inhibit” typically means a decrease by at least 10% as compared to an appropriate control (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” does not encompass a complete inhibition or reduction as compared to a reference level. “Complete inhibition” is a 100% inhibition as compared to an appropriate control.

The terms “increase”, “enhance”, or “activate” are all used herein to mean an increase by a reproducible, statistically significant amount. In some embodiments, the terms “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, a 20 fold increase, a 30 fold increase, a 40 fold increase, a 50 fold increase, a 6 fold increase, a 75 fold increase, a 100 fold increase, etc. or any increase between 2-fold and 10-fold or greater as compared to an appropriate control. In the context of a marker, an “increase” is a reproducible statistically significant increase in such level.

As used herein, a “reference level” refers to a normal, otherwise unaffected cell population or tissue (e.g., a biological sample obtained from a healthy subject, or a biological sample obtained from the subject at a prior time point, e.g., a biological sample obtained from a patient prior to being diagnosed with an asthma or an allergic disease, or a biological sample that has not been contacted with an agent disclosed herein).

As used herein, an “appropriate control” refers to an untreated, otherwise identical cell or population (e.g., a patient who was not administered an agent described herein, or was administered by only a subset of agents described herein, as compared to a non-control cell).

The term “statistically significant” or “significantly” refers to statistical significance and generally means a two standard deviation (2SD) or greater difference.

As used herein the term “comprising” or “comprises” is used in reference to compositions, methods, and respective component(s) thereof, that are essential to the method or composition, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### BRIEF DESCRIPTION OF THE DRAWINGS

FIGS. 1A-1H show Notch4 expression on lung T_{reg} cells in allergic airway inflammation. (FIG. 1A) RT-PCR of Notchl-4 transcripts in lung T_{reg} and Tₑff cells isolated from PBS, OVA and OVA+UFP mouse groups. (FIG. 1B and FIG. 1C) Flow cytometric analysis, cell frequencies and mean fluorescence intensity (MFI) of Notch4 expression on lung T_{reg} and Tₑff cells in the respective mouse groups. (FIG. 1D and FIG. 1E) Flow cytometric analysis and cell frequencies of Notch4 expression on OT-II⁺CD4⁺Foxp3⁺ T cells generated in co-cultures with sham or OVA₃₂₃₋₃₃₉+UFP-pulsed alveolar macrophages without or with IL-6 or anti-IL-6R mAb. (FIG. 1F) Flow cytometric analysis and cell frequencies of Notch4 expression in Helios^{high} and Helios^{low} lung T_{reg} cells isolated from the respective mouse groups. (FIG. 1G) Flow cytometric analysis and cell frequencies of Notch4 expression on in vitro differentiated T_{reg} cells derived from naive CD4⁺ T cells isolated from Foxp3^{YFPCre}, Foxp3^{YFPCre}Il6r^{Δ/Δ} and Foxp3^{YFPCre}Stat3^{Δ/Δ} mice and either untreated or treated with IL-6. (FIG. 1H) ChIP assays for the binding of STAT3 and control (IgG) antibodies to the Notch4 promoter in lung T_{reg} cells of OVA+UFP-treated Foxp3^{YFPCre}, and Foxp3^{YFPCre}Stat3^{Δ/Δ} mice. Each symbol represents one mouse. Numbers in flow plots indicate percentages. Error bars indicate SEM. Statistical tests for all panels: One-way ANOVA with Dunnett’s post hoc analysis. *P<0.05, **P<0.01, ***P<0.001, ****P<0.0001. Data representative of two or three independent experiments. (FIGS. 1A, 1D to 1F) n=5, (FIGS. 1B and 1C) n=15 and (FIGS. 1G and 1H) n=6 replicates per group.

FIGS. 2A-2I show Notch4 expression on lung T_{reg} cells licenses allergic airway inflammation. (FIG. 2A) Representative PAS-stained sections of lung tissues isolated from Foxp3^{YFPCre}, CD4^{Cre}Notch4^{Δ/Δ} or Foxp3^{YFPCre}Notch4^{Δ/Δ} mice segregated into PBS, OVA or OVA+UFP-treated groups (200X magnification). (FIG. 2B) Inflammation scores in the respective lung tissues. (FIG. 2C) AHR in the respective mouse groups in response to methacholine. (FIG. 2D and FIG. 2E) serum total and OVA-specific IgE concentrations. (FIG. 2F and FIG. 2G), absolute numbers of lung CD4⁺ T cells and eosinophils. (FIG. 2H and FIG. 2I) IL-13 and IL-17 expression in lung Foxp3⁺CD4⁺ T_{reg} (FIG. 2H) and Foxp3⁻CD4⁺T_{eff} cells (FIG. 2I). Each symbol represents an independent sample. Numbers in flow plots indicate percentages. Error bars indicate SEM. Statistical tests: two-way ANOVA with Sidak’s post hoc analysis (FIGS. 2B-2I). ***P<0.001, ****P<0.0001. Data representative of two or three independent experiments. n=5-15 mice per group.

FIGS. 3A-3F show Notch4-dependent transcriptional programs in lung T_{reg} cells. (FIG. 3A) Volcano plot of differential gene expression in Foxp3^{YFPCre} versus Foxp3^{YFPCre}Notch4^{Δ/Δ} Treg cells. FDR, false discovery rate; log2FC, log2(fold change). (FIG. 3B) Enrichment pathway analysis of Hippo and Wnt pathways. (FIG. 3C and FIG. 3D) AHR in the respectively treated Foxp3^{YFPCre}Wwtr1^{Δ/Δ}Yapl^{Δ/Δ} (FIG. 3C) and Foxp3^{YFPCre}Ctnnbl^{Δ/Δ} mice (FIG. 3D) compared to control Foxp3^{YFPCre} mice in response to methacholine. (FIG. 3E and FIG. 3F) Absolute lung tissue eosinophils and IL-13⁺ and IL-17⁺ Tₑff cells in Foxp3^{YFPCre}Wwtrl^{Δ/Δ}Yapl^{Δ/Δ} (FIG. 3E) and Foxp3^{YFPCre}Ctnnbl^{Δ/Δ} mice (FIG. 3F) compared to control Foxp3^{YFPCre} mice. Each symbol represents an independent sample. Numbers in flow plots indicate percentages. Error bars indicate SEM. Statistical tests: two-way ANOVA with Sidak’s post hoc analysis (FIG. 3C to FIG. 3F). *P<0.05, **P<0.01, ***P<0.001, ****P<0.0001. Data representative of two or three independent experiments. (FIG. 3A and FIG. 3B): n=4-5 and (FIGS. 3C-3F) n=10 mice per group.

FIGS. 4A-4H show Notch4 destabilizes T_{reg} cell in a Hippo pathway-dependent manner. (FIG. 4A) Flow cytometric analysis and frequencies of exT_{reg} (GFP⁺YFP⁻) cells, plotted as a fraction of exT_{reg} to total T_{reg} (YFP⁺) cells in lung tissue. (FIG. 4B and FIG. 4C) Flow cytometric analysis and frequencies of IL-13 (FIG. 4B) and IL-17 (FIG. 4C) -producing exT_{reg} cells in lung tissues. (FIG. 4D) Methylation status of CpG motifs in T_{reg} cells isolated from lung tissue of sham versus OVA+UFP-sensitized and challenged mice of the respectively indicated genotypes. Numbers on the left side indicate the position of the respective motifs. (FIG. 4E) Global methylation status of Foxp3 CNS2 in the respective T_{reg} cell populations. (FIG. 4F to FIG. 4H) In vitro suppression of the proliferation of WT responder CD4⁺ T cells (T_{eff}) by the respective T_{reg} cell populations. Each symbol represents an independent sample. Numbers in flow plots indicate percentages. Error bars indicate SEM. Statistical tests: two-way ANOVA with Sidak’s post hoc analysis (FIGS. 4A to 4C and 4G to 4H); One-way ANOVA with Dunnett’s post hoc analysis (FIG. 4E). * **P<0.01, ***P<0.001, ****P<0.0001. Data representative of two or three independent experiments. n=4-6 mice per group.

FIGS. 5A-5L show Notch4 promotes ILC2 activation via a GDF15-dependent mechanism (FIG. 5A) Flow cytometric analysis and frequencies of IL13⁺ ILC2 (Lineage⁻ T1/ST2⁺ cells) in mice of respective genotypes treated as indicated. (FIG. 5B and FIG. 5C) In vitro suppression assays of ILC2 cells isolated from OVA+UFP-treated Foxp3^{YFPCre} mice and incubated with T_{reg} cells of mice of the respective genotypes treated as indicated. Additional treatment of in vitro cultures with isotype control or anti-Notch4 mAb is also indicated. (FIG. 5D) Flow cytometric analysis and frequencies of GDF15⁺ lung T_{reg} cells of mice of the respective genotypes treated as indicated. (FIG. 5E) Flow cytometric analysis and frequencies of IL-13 expression in cultures of naive ILC2 stimulated with IL-33, GDF15 or both. (FIG. 5F) IL-13 expression in naive ILC2 incubated with Notch4^{high} T_{reg} cells from OVA+UFP treated mice without or with blocking GDF15 peptide. (FIG. 5G and FIG. 5H) AHR and IL13⁺ ILC2 frequencies in Foxp3^{YFPCre} mice treated as indicated and challenged in the presence of carrier protein or GDF15 blocking peptide. (FIG. 5I to FIG. 5K) AHR and frequencies of the indicated lung cell types in sham or OVA+UFP sensitized and challenged Foxp3^{YFPCre} and Foxp3^{YFPCre}Notch4^{Δ/Δ} mice treated with recombinant GDF15, as indicated. Each symbol represents an independent sample. Numbers in flow plots indicate percentages. Error bars indicate SEM. Statistical tests: two-way ANOVA with Sidak’s post hoc analysis (FIGS. 5A to 5D, 5G and 5I); One-way ANOVA with Dunnett’s post hoc analysis (FIGS. 5E and 5F, 5H and 5J). *P<0.05, ***P<0.001, ****P<0.0001. Data representative of two or three independent experiments. n=5-15 mice per group.

FIGS. 6A-6H show Notch4 expression on circulating T_{reg} cells segregates with asthma severity. (FIGS. 6A and 6B) Flow cytometric analysis, cell frequencies and MFI of Notch4 expression on circulating T_{reg} cells (FIG. 6A) and T_{eff} cells (FIG. 6B) of control and asthmatic subjects, the latter segregated for asthma severity. (FIG. 6C) Flow cytometric analysis, cell frequencies and MFI of Notch4 expression on Helios^{high} versus Helios^{low} circulating T_{reg} cells of control and asthmatic subjects. (FIG. 6D and FIG. 6E) Flow cytometric analysis, cell frequencies and MFI of Yap (FIG. 6D) and beta-catenin (FIG. 6E) expression on circulating T_{reg} cells of control and severe asthmatic subjects. (FIG. 6F) Serum GDF15 concentrations in asthmatic subjects plotted as a function of Notch4 expression on circulating T_{reg} cells. (FIGS. 6G and 6H). In vitro suppression third party CD4⁺ T cells (T_{eff}) by the Notch4^{high} versus Notch4^{low} T_{reg} cells from severe asthmatics compared to T_{reg} cells of control subjects. Each symbol represents an independent sample. Numbers in flow plots indicate percentages. Error bars indicate SEM. Statistical tests: One-way ANOVA with Dunnett’s post hoc analysis (FIGS. 6A to 6E); two-way ANOVA with Sidak’s post hoc analysis (FIG. 6G); **P<0.01, ****P<0.0001. Data representative of two or three independent experiments. n= 11-41 probands per group (FIGS. 6A, 6B) n= 11-24 (FIGS. 6C to 6E) n= 21 (FIG. 6F) n = 4 (FIG. 6G).

FIGS. 7A-7E show Notch4 expression on lung T_{reg} cells in allergic airway inflammation. (FIGS. 7A to 7C) Flow cytometric analysis, cell frequencies and mean fluorescence intensity (MFI) of Notch1, 2 and 3 expression on lung T_{reg} and T_{eff} cells in the respective mouse groups. (FIG. 7D) Cell frequencies of Notch4 expression on OT-II⁺CD4⁺Foxp3⁺ T cells generated in co-cultures with sham or OVA₃₂₃₋₃₃₉+UFP-pulsed alveolar macrophages without or with IL-1B, IL-25, IL-33, TSLP or TNFa. (FIG. 7E) ChIP assays for the binding of STAT3 and control (IgG) antibodies to the Notch1, 2 and 3 promoters in lung T_{reg} cells of OVA+UFP-treated Foxp3^{YFPCre}, and Foxp3^{YFPCre}Stat3^{Δ/Δ} mice. Each symbol represents one mouse. Numbers in flow plots indicate percentages. Error bars indicate SEM. Statistical tests for all panels: One-way ANOVA with Dunnett’s post hoc analysis. *P<0.05, **P<0.01, ***P<0.001, ****P<0.0001. Data representative of two or three independent experiments. N = 5-10 replicas per group.

FIGS. 8A-8D Notch4 expression on lung T_{reg} cells licenses allergic airway inflammation. (FIG. 8A) RT-PCR analysis of Notch4 expression in CD4^{Cre} mice in B-cells and T-cells. (FIG. 8B) RT-PCR analysis of Notch4 expression in Foxp3^{YFPCre} mice in both T_{reg} and T_{eff} cells. (FIGS. 8C and 8D) IL-4 and IFN□ expression in lung Foxp3⁺CD4⁺ T_{reg}. (FIG. 8C) and Foxp3⁻CD4⁺T_{eff} cells. (FIG. 8D) derived from the respectively treated Foxp3^{YFPCre}, CD4^{Cre}Notch4^{Δ/Δ} and Foxp3^{YFPCre}Notch4^{Δ/Δ} mice. Each symbol represents one mouse. Numbers in flow plots indicate percentages. Error bars indicate SEM. Statistical tests for all panels: One-way ANOVA with Dunnett’s post hoc analysis. ****P<0.0001. Data representative of two or three independent experiments. (FIGS. 8A and 8B) N = 10 replicas per group; (FIGS. 8C and 8D) n= n=5-15 mice per group.

FIGS. 9A-9C show Superior function of Notch4-deficient OTII⁺ iT_{reg} in suppressing airway inflammation. (FIG. 9A) Airway hyperresponsiveness in Foxp3^{YFPCre} sensitized either with PBS or OVA, then challenged with OVA+UFP following transfer of OTII+Foxp3^{YFPCre} or OTII⁺Fox3^{YFPCre}Notch4^{Δ/Δ} iT_{reg} cells. (FIG. 9B) Eosinophils numbers for the respective mouse groups. (FIG. 9C) IL-4, IL-13, IL-17 and IFN_{Y} expression in lung Foxp3⁻CD4⁻ Tₑff cells. Error bars indicate SEM. Statistical tests. two-way ANOVA with Sidak’s post hoc analysis (FIG. 9A) One-way ANOVA with Dunnett’s post hoc analysis. (FIG. 9B and FIG. 9C). *P<0.05, **P<0.01, ***P<0.001, ****P<0.0001. Data representative of two or three independent experiments. n=5-10 mice per group.

FIGS. 10A-10I show allergic airway inflammatory responses in mice with T_{reg} cell-specific Pofut1 or Rbpjl deletion. (FIG. 10A) Representative PAS-stained sections of lung tissues isolated from Foxp3^{YFPCre}, Foxp3^{YFPCre}Pofutl^{Δ/Δ} or Foxp3^{YFPCre}Rbpjl^{Δ/Δ} mice segregated into PBS, OVA or OVA+UFP-treated groups (200X magnification). (FIG. 10B) Inflammation scores in the respective lung tissues. (FIG. 10C) AHR in the respective mouse groups in response to methacholine. (FIG. 10D and FIG. 10E) serum total and OVA-specific IgE concentrations. (FIG. 10F and FIG. 10G), absolute numbers of lung CD4⁺ T cells and eosinophils. (FIG. 10H and FIG. 10I) IL-4, IL-13, IL-17 and IFN_{Y} expression in lung Foxp3⁺CD4⁺ T_{reg} (FIG. 10H) and Foxp3⁻CD4⁺T_{eff} cells (FIG. 10I). Each symbol represents an independent sample. Error bars indicate SEM. Statistical tests. two-way ANOVA with Sidak’s post hoc analysis (FIG. 10C) One-way ANOVA with Dunnett’s post hoc analysis. (FIGS. 10A, 10D-10G). ***P<0.001, ****P<0.0001. Data representative of two or three independent experiments. n=5-10 mice per group.

FIGS. 11A-11H show allergic airway inflammatory responses in mice with T_{reg} cell-specific Notch1 or Notch2 deletion or global Notch3 deletion. (FIGS. 11A to 11C) Airway hyperresponsivness in Foxp3^{YFPCre}, Foxp3^{YFPCre}Notchl^{Δ/Δ}, Foxp3^{YFPCre}Notch2^{Δ/Δ}, or Foxp3^{YFPCre}Notch3^{-/-} mice segregated into PBS, OVA or OVA+UFP-treated groups (200X magnification). (FIG. 11D) serum OVA-specific IgE concentrations. (FIG. 11E and FIG. 11F), absolute numbers of lung CD4⁺ T cells and eosinophils. (FIG. 11G and FIG. 11H) IL-4, IL-13, and IL-17 expression in lung Foxp3⁻CD4⁺ T_{eff} (FIG. 11G) and Foxp3⁺CD4⁺T_{reg} cells (FIG. 11H). Each symbol represents an independent sample. Error bars indicate SEM. Statistical tests: two-way ANOVA with Sidak’s post hoc analysis (FIGS. 11A-11C). One-way ANOVA with Dunnett’s post hoc analysis. (FIGS. 11D-11H). Data representative of two or three independent experiments. n=5 mice per group.

FIGS. 12A-12I show T_{reg} cell-specific Il6r and stat3 deletions attenuate allergic airway inflammation. (FIG. 12A) Representative PAS-stained sections of lung tissues isolated from Foxp3^{YFPCre}, Foxp3^{YFPCre}Il6r^{Δ/Δ} or Foxp3^{YFPCre}Stat3^{Δ/Δ} mice segregated into PBS, OVA or OVA+UFP-treated groups (200X magnification). (FIG. 12B) Inflammation scores in the respective lung tissues. (FIG. 12C) AHR in the respective mouse groups in response to methacholine. (FIG. 12D and FIG. 12E) serum total and OVA-specific IgE concentrations. (FIG. 12F and FIG. 12G), absolute numbers of lung CD4⁺ T cells and eosinophils. (FIG. 12H and FIG. 12I) IL-13 and IL-17 expression in lung Foxp3⁺CD4⁺ T_{reg} (FIG. 12H) and Foxp3⁻CD4⁺ T_{eff} cells (FIG. 12I). Each symbol represents an independent sample. Error bars indicate SEM. Statistical tests: two-way ANOVA with Sidak’s post hoc analysis (FIG. 12C). One-way ANOVA with Dunnett’s post hoc analysis. (FIGS. 12B, 11D-11I) *P<0.05, **P<0.01, ***P<0.001, ****P<0.0001. Data representative of two or three independent experiments. n=5-10 mice per group.

FIGS. 13A-13J show T_{reg} cell-specific Notch4 deletion rescues HDM induced allergic airway inflammation. (FIG. 13A) scheme of the house dust mite airway inflammation protocol (FIG. 13B) Representative PAS-stained sections of lung tissues isolated from Foxp3^{YFPCre} or Foxp3^{YFPCre}Notch4^{Δ/Δ} mice segregated into PBS, OVA or OVA+UFP-treated groups (200X magnification). (FIG. 13C) Inflammation scores in the respective lung tissues. (FIG. 13D) AHR in the respective mouse groups in response to methacholine. (FIG. 13E) serum total IgE concentrations. (FIG. 13F to FIG. 13H), absolute numbers of lung CD4⁺ T cells, neutrophils and eosinophils. (FIG. 13I and FIG. 13J) IL-4, IL-13, IL-17 and IFN_{Y} expression in lung Foxp3⁺CD4⁺ T_{reg} (FIG. 13I) and Foxp3⁻CD4⁺ T_{eff} cells (FIG. 13J). Each symbol represents an independent sample. Numbers in flow plots indicate percentages. Error bars indicate SEM. Statistical tests: two-way ANOVA with Sidak’s post hoc analysis (FIG. 13D). One-way ANOVA with Dunnett’s post hoc analysis. (FIGS. 13C, 13E-13K), **P<0.01, ***P<0.001, ****P<0.0001. Data representative of two or three independent experiments. n=5 mice per group.

FIGS. 14A-14H show T_{reg} cell-specific Notch4 deletion rescues chronic allergic airway inflammation. (FIG. 14A) Scheme for the chronic airway inflammation mouse protocol (FIG. 14B) Representative Sirius-Red-stained sections of lung tissues isolated from Foxp3^{YFPCre} or Foxp3^{YFPCre}Notch4^{Δ/Δ} mice segregated into PBS, OVA or OVA+UFP-treated groups (200X magnification). (FIG. 14C) Collagen disposition measurement in the respective lung tissues. (FIG. 14D) AHR in the respective mouse groups in response to methacholine. (FIG. 14E and FIG. 14F), absolute numbers of lung CD4⁺ T cells, neutrophils and eosinophils. (FIG. 14G and FIG. 14H) IL-4, IL-13, and IL-17 expression in lung Foxp3⁺CD4⁺ T_{reg} (FIG. 14G) and Foxp3⁻CD4⁺T_{eff} cells (FIG. 14H). Serum OVA-specific IgE titers in the respective groups. Each symbol represents an independent sample. Error bars indicate SEM. Statistical tests: two-way ANOVA with Sidak’s post hoc analysis (FIG. 14D). One-way ANOVA with Dunnett’s post hoc analysis. (C, E-I), *P<0.05, ****P<0.0001. Data representative of two or three independent experiments. n=5 mice per group.

FIGS. 15A-15F show gating strategy for Notch4 high versus Notch4 low T_{reg} cells. (FIG. 15A and FIG. 15B) Forward and side scatter (FSC and SSC) analysis of lung mononuclear cells. (FIG. 15C) Amcyan viability dye versus forward scatter. (FIG. 15D) CD4 versus forward scatter gated on CD4+ live cells. (FIG. 15E) Foxp3 versus CD4 staining, gated on Foxp3⁺ T_{reg} cells. (FIG. 15F) Foxp3 versus Notch4 staining, gated on T_{reg} cells showing high versus low gates used for sorting of T_{reg} cells.

FIGS. 16A and 16B show Th cytokine expression in lung T_{reg} cells of Foxp3^{YFPCre}Yap1^{Δ/Δ}Wwtr1^{Δ/Δ} and Foxp3^{YFPCre}Ctnnb1^{Δ/Δ} mice in airway inflammation. (FIGS. 16A and 16B) IL-4, IL-13, IL-17 and IFN□ expression in lung Foxp3⁺CD4⁺ T_{reg} cells. Each symbol represents an independent sample. Error bars indicate SEM. Statistical tests: One-way ANOVA with Dunnett’s post hoc analysis. (FIGS. 16A and 16B). *P<0.05, **P<0.01, ***P<0.001, ****P<0.0001. Data representative of two or three independent experiments. n=5-10 mice per group.

FIGS. 17A-17I show gating strategy for GDF15 staining in different cell types (FIG. 17A and FIG. 17B) Forward and side scatter (FSC and SSC) analysis of lung mononuclear cells. (FIG. 17C) Amcyan viability dye versus forward scatter. (FIG. 17D) CD4 versus CD45 gated on CD4⁺ or CD4⁻live cells. (FIG. 17E) Foxp3 versus GDF15 staining, gated on CD4⁺ cells. (FIG. 17F) GDF15 versus CD45 staining, gated on CD45⁻cells (FIG. 17G) F4/80 versus CD11c staining, gated on CD45⁺CD4⁻ cells to show interstitial macrophages in A and alveolar macrophages in B. (FIG. 17H) F4/80 versus GDF15 staining, gated on B (FIG. 17I) CD11c versus GDF15 staining, gated on A.

FIGS. 18A-18E GDF15 restores airway inflammation in Foxp3^{YFPCre}Notch4^{Δ/Δ} mice. (FIG. 18A) Representative PAS-stained sections of lung tissues isolated from Foxp3^{YFPCre} and Foxp3^{YFPCre}Notch4^{Δ/Δ} with either PBS or OVA+UFP, the latter either alone or supplemented with GDF15, as indicated (200X magnification). (FIG. 18B) Inflammation score for the respective mouse groups. (FIG. 18C) Frequencies of ILC in the lungs of respective mouse groups. (FIG. 18D) IL-4, IL-13, and IL-17 expression in lung Foxp3⁻CD4⁺ T_{eff} cells in the respective groups. (FIG. 18E) IL-4, IL-13, and IL-17 expression in lung Foxp3⁺CD4⁺ T_{reg} cells in the respective groups. Error bars indicate SEM. Statistical tests. One-way ANOVA with Dunnett’s post hoc analysis. (FIGS. 18A-18E) **P<0.01, ***P<0.001, ****P<0.0001. Data representative of two or three independent experiments. n=10 mice per group.

FIGS. 19A-19G show a GDF15 blocking peptide attenuates allergic airway inflammation. (FIG. 19A) Representative PAS-stained sections of lung tissues isolated from Foxp3^{YFPCre}, treated with GDF15 blocking peptide segregated into PBS, or OVA+UFP-treated groups (200X magnification). (FIG. 19B) Inflammation score for the respective mouse groups. (FIG. 19C) Frequencies of ILC in the respective mouse groups. (FIG. 19D and FIG. 19E) Eosinophils and lymphocytes numbers in the respective mouse groups. (FIG. 19F) IL-4, IL-13, and IL-17 expression in lung Foxp3⁻CD4⁺ T_{eff} cells in the respective groups. (FIG. 19G) IL-4, IL-13, and IL-17 expression in lung Foxp3⁺CD4⁺ T_{reg} cells in the respective groups. Error bars indicate SEM. Statistical tests. One-way ANOVA with Dunnett’s post hoc analysis. (FIGS. 19A-19E) **P<0.01, ***P<0.001, ****P<0.0001. Data representative of two or three independent experiments. n=10 mice per group.

FIGS. 20A-20D show Notch receptor expression in human T_{reg} and T_{eff} cells. (FIG. 20A) Flow cytometric analysis, cell frequencies and mean fluorescence intensity (MFI) of Notch1, 2 and 3 expression in peripheral blood T_{reg} cells (FIG. 20A) and T_{eff} cells (FIG. 20B) of control and asthmatic subjects, the latter segregated for asthma severity. (FIG. 20C) Flow cytometric analysis and cell frequencies of Notch4 peripheral blood T_{reg} cells of healthy control, food allergy, eczema and food allergy+eczema (FIG. 20D) Serum GDF15 concentrations in asthmatic subjects plotted as a function of Notch4 expression on circulating T_{reg} cells. Error bars indicate SEM. Statistical tests. One-way ANOVA with Dunnett’s post hoc analysis. (FIGS. 20A-20C), **P<0.01, ***P<0.001, ****P<0.0001. Data representative of two or three independent experiments. n= 11-44 probands per group (FIGS. 20A to 20D).

### DETAILED DESCRIPTION

### Treating or Preventing Asthma or an Allergic Disease

Elucidating the mechanisms that sustain asthmatic inflammation is critical for identifying therapeutics for treating and/or preventing asthma. Data presented herein show that IL-6 and STAT3-dependent upregulation of Notch4 on lung tissue regulatory T (T_{reg}) cells is critical for allergens and particulate matter pollutants to promote airway inflammation. Notch4 subverted T_{reg} cells into Th2 and Th17 effector T (T_{eff}) cells by Wnt and Hippo pathway-dependent mechanisms, respectively. Wnt activation induced GDF15 expression in T_{reg} cells, which activated group 2 innate lymphoid cells (ILC2) to provide a feed-forward mechanism for aggravated inflammation. Notch4, Wnt and Hippo were upregulated on circulating T_{reg} cells of asthmatics as a function of disease severity, in association with reduced T_{reg} cell-mediated suppression.

Accordingly, provided herein are methods for treating a subject having asthma or an allergic disease, or preventing asthma or an allergic disease in a subject at risk of developing such disease or disorder, comprising administering to the subject an effective amount of an agent that inhibits Wnt signaling. For example, an agent that inhibits any component of the Wnt signaling pathway.

Provided herein are methods for treating a subject having asthma or an allergic disease, or preventing asthma or an allergic disease in a subject at risk of developing such disease or disorder, comprising administering to the subject an effective amount of an agent that inhibits Hippo signaling. For example, an agent that inhibits any component of the Hippo signaling pathway.

Provided herein are methods for treating a subject having asthma or an allergic disease, or preventing asthma or an allergic disease in a subject at risk of developing such disease or disorder, comprising administering to the subject an effective amount of an agent that inhibits GFD15. For example, an agent that inhibits the GDF15 gene or gene product expression level or activity.

In one embodiment, the subject having or at risk of having asthma or an allergic disease is further administered a Notch4 inhibitor, e.g., an anti-Notch4 antibody.

In one embodiment, the subject having or at risk of having asthma or an allergic disease is administered a combination of agents that inhibit a target, e.g., Wnt signaling, Hippo signaling, GDF15, and/or Notch4, described herein. Exemplary combinations of agents that inhibit a target are described herein in Table 1. The combinations presented in Table 1 are not meant to be limiting. In Table 1, “X” indicates the given inhibitor is included in the combination of agents.

TABLE 1

Combinations of Inhibitors for treatment and/or prevention of asthma or allergic disease.

| | | | |
|---|---|---|---|
| Wnt Signaling Inhibitor | Hippo Signaling Inhibitor | GDF15 Inhibitor | Notch4 Inhibitor |
| x | x | | |
| X | | X | |
| X | | | X |
| X | X | X | |
| X | | X | X |
| X | X | | X |
| X | X | X | X |
| | X | X | |
| | X | | X |
| | X | X | X |
| | | X | X |

In methods described herein, a Notch4 inhibitor is never used as a monotherapy. Similarly, no composition described herein comprises, consists of or consists essentially of only a Notch4 inhibitor. According, in one embodiment, the agent is not a Notch4 inhibitor administered as a monotherapy. Further, in one embodiment, wherein a composition comprises only one agent described herein, that agent is not a Notch4 inhibitor.

As used herein, an “asthma” refers to a disease characterized by inflammation in the airways of the lungs, reversible airways obstructions, bronchospasms, wheezing, coughing, tightness of the chest, and shortness of breath. Asthma is thought to be caused by environmental and genetic factors, include, but not limited to exposure to air pollutants and allergens, aspirin and beta blockers, and a family history of asthma.

Asthma is classified by the frequency of symptoms, the severity of symptoms, forced expiratory volume in one second (FEV1), and peak expiratory flow rate. Asthma can further be classified based on the subject’s response to a medication, e.g., atopic or non-atopic, wherein atropic refers to a predisposition towards developing a type 1 hypersensitivity.

In various embodiments, the asthma is allergic asthma (e.g., induced by exposure to allergens), asthma without allergies (e.g., induced by an upper respiratory infection, such as a cold, flu, or rhinovirus), aspirin exacerbated respiratory disease (e.g., induced by the intake of aspirin), exercised-induced asthma, cough variant (e.g., characterized by a dry, hacking cough), or occupational asthma (e.g., induced by an irritant a subject is exposed to on a job, for example, a fire fighter is exposed to smoke, and can experience smoke-inhalation, while performing their job). A skilled clinician can identify a type of asthma a subject has, or is at risk of having (e.g., a fire fighter would be at risk of having occupational asthma), using standard techniques know in the art, e.g., those methods described herein below.

As used herein, an “allergic disease” is a disease that is characterized by an immune system response to an otherwise harmless substance in the environment. For example, when a subject who has an allergic disease is exposed to common environmental substances the subject’s B lymphocytes produce specific antibodies against that substance, resulting in an immune response. Exemplary substances that, e.g., can cause an allergic disease include dust mites, pollen (e.g., from plants, trees, flowers, or grass), animal dander (e.g., from domestic or farm animals), mold, food (e.g., tree nuts, peanuts, shellfish, fish, milk, eggs, or wheat), and latex. A child whose parent(s) or sibling(s) have allergies are at an increased risk of developing an allergic disease. The specific cause of an allergic diseases (e.g., what the allergen is) can be identified by a skilled clinician using common techniques, e.g., skin prick tests and radioallergosorbent tests.

In one embodiment, the allergic disease is allergic rhinitis, sinusitis, otitis media, atopic dermatitis (e.g., eczema), urticaria, angioedema, and anaphylaxis.

A subject can be identified as having, e.g., asthma or an allergic reaction, by a skilled clinician. Diagnostic tests useful in identifying a subject having asthma or an allergic disease are known in the art, and further described herein.

As used herein a subject “at risk of having asthma or an allergic disease” refers to a subject who is in contact, or potentially in contact, with known asthma triggers (e.g., factors that can result in the onset of asthma). Non-limiting factors that can, e.g., trigger the onset of asthma or allergic disease, include airborne substances, (e.g., pollen, dust mites, mold spores, pet dander or particles of cockroach waste); respiratory infections, (e.g., the common cold, pneumonia); physical activity (e.g., can trigger exercised-induced asthma); cold air; air pollutants and irritants, (e.g., smoke and cigarette smoke); certain medications (e.g., blockers, aspirin, ibuprofen (Advil, Motrin IB, others) and naproxen (Aleve)); strong emotions or stress; sulfites and preservatives added food and/or beverages (e.g., found in shrimp, dried fruit, processed potatoes, beer, and wine); and gastroesophageal reflux disease (GERD). A subject is also considered at risk of asthma or an allergic disease if the subject has a family history of asthma or an allergic disease (e.g., if an immediate family member has had asthma or an allergic disease).

Additionally provided herein is a method for treating asthma or an allergic disease comprising: (a) obtaining a biological sample from a subject; (b) measuring the level of Notch4 in the biological sample of (a); (c) comparing the level of (b) with a reference level, wherein a subject is identified as having asthma or an allergic disease if the level of (b) is greater than a reference level; and (d) administering to the subject identified as having at risk asthma or an allergic disease any of the agents or compositions thereof described herein.

Further provided herein method for preventing asthma or an allergic disease comprising: (a) obtaining a biological sample from a subject; (b) measuring the level of Notch4 in the biological sample of (a); (c) comparing the level of (b) with a reference level, wherein a subject is identified as being at risk of having asthma or an allergic disease if the level of (b) is greater than a reference level; and (d) administering to the subject identified as having at risk asthma or an allergic disease any of the agents or compositions thereof described herein.

In one embodiment, the levels of Notch4 are measured in vitro, or ex vivo. The levels of Notch4 in the sample can be measured using standard techniques, e.g., FACS analysis, or immunofluorescence. Protein and mRNA levels of Notch4 can be assessed using western blotting or PCR-based assays, respectively, as described herein. In one embodiment, the level of Notch4 that is increased at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, at least 70-fold, at least 80-fold, at least 90-fold, at least 100-fold, or more as compared to the reference level, or at least 10%, at least 20%, at least 30%, at least 40%, at least 50%, at least 60%, at least 70%, at least 80%, at least 90%, or at least 99% or more as compared to the reference level. The reference level can be the level of Notch4 in a sample obtained from a healthy subject, e.g., a subject who is not at risk of asthma or an allergic reaction.

In one embodiment, the biological sample is a blood sample, a peripheral blood sample, a sputum sample, a lung tissue sample, a lung biopsy sample, or a bronchial lavage sample. In one embodiment, the biological sample is any sample that contains alveolar macrophages. In one embodiment, the biological sample is taken from a subject that has previously been diagnosed with asthma or an allergic disease. In one embodiment, the biological sample is taken from a subject that has previously been diagnosed with and treated for asthma or an allergic disease. In one embodiment, the biological sample is taken from a subject that has not been diagnosed with asthma or an allergic disease. Methods for collecting samples from a subject are known in art and can be performed by a skilled person.

### Wnt Signaling Pathway

Wnt proteins regulate the proliferation of cells via intercellular signaling. Wnt signals are active in numerous contexts, initially in early development and later during the growth and maintenance of various tissues. In comparison to other growth factors, Wnt signals have several unique properties, including a short range of action. Thereby, Wnts predominantly mediate signaling locally, between neighboring cells. In addition, Wnt signals give shape to tissues as cells are proliferating. This is a consequence of the ability of Wnt signaling to confer polarity and asymmetry to cells. Wnt proteins are highly conserved in evolution and are active in every branch of the animal kingdom.

Wnt signaling is often implicated in stem cell control, as a proliferative and self-renewal signal. Mutations in Wnt genes or Wnt pathway components lead to specific developmental defects, while various human diseases, including cancer, are caused by abnormal Wnt signaling.

Insights into the mechanisms of Wnt action have emerged from several systems: genetics in Drosophila and Caenorhabditis elegans; biochemistry in cell culture and ectopic gene expression in Xenopus embryos. As currently understood, Wnt proteins bind to receptors of the Frizzled and LRP families on the cell surface. Through several cytoplasmic relay components, the signal is transduced to ß-catenin, which enters the nucleus and forms a complex with TCF to activate transcription of Wnt target genes.

Components of the Wnt signaling pathway include, but are not limited to Frizzled (SFRP), Dishevelled (Dvl), TCF/Lef, LRP, APC, Axin, β-Catenin, Dickkopf, and GSK3. Homologs, paralogs, and/or orthologs of components of the Wnt signaling pathway are known in the art and can be readily identified by one skilled in the art. The Wnt Signaling pathway is further reviewed in, e.g., Routledge D, Scholpp, S. Mechanisms of intercellular Wnt transport. Development. 2019 May 15;146(10). pii: dev176073. doi: 10.1242/dev.176073; Schaefer KN, Peifer M. Wnt/Beta-Catenin Signaling Regulation and a Role for Biomolecular Condensates. Dev Cell. 2019 Feb 25;48(4):429-444; Tian, A.; Benchabane, H.; Ahmed, Y. Wingless/Wnt Signaling in Intestinal Development, Homeostasis, Regeneration and Tumorigenesis: A Drosophila Perspective. J. Dev. Biol. 2018, 6, 8; Katrin E. Wiese Roel Nusse and Renee van Amerongen. Wnt signalling: conquering complexity. Development (2018) 145, dev165902. doi:10.1242/dev.165902; Steinhart Z, Angers S. Wnt signaling in development and tissue homeostasis. Development. 2018 Jun 8;145(11). pii: dev146589. doi: 10.1242/dev.146589; Bejsovec A. Wingless Signaling: A Genetic Journey from Morphogenesis to Metastasis. Genetics. 2018 Apr;208(4):1311-1336. doi: 10.1534/genetics.117.300157; and Ashley Ceinwen Humphries, Marek Mlodzik, From instruction to output: Wnt/PCP signaling in development and cancer, Current Opinion in Cell Biology, Volume 51, April 2018, Pages 110-116; the contents of which are incorporated herein by reference in their entireties.

### Hippo Signaling Pathway

The Hippo signaling pathway, also known as the Salvador-Warts-Hippo (SWH) pathway, controls organ size in animals through the regulation of cell proliferation and apoptosis. The pathway takes its name from one of its key signaling components—the protein kinase Hippo (Hpo). Mutations in this gene lead to tissue overgrowth, or a “hippopotamus”-like phenotype.

The Hippo signaling pathway is involved in restraining cell proliferation and promoting apoptosis. As many cancers are marked by unchecked cell division, this signaling pathway has become increasingly significant in the study of human cancer. Hippo pathway also has critical role in stem cell and tissue specific progenitor cell self-renewal and expansion.

The Hippo signaling pathway appears to be highly conserved. While most of the Hippo pathway components were identified in the fruit fly (Drosophila melanogaster) using mosaic genetic screens, orthologs to these components (genes that function analogously in different species) have subsequently been found in mammals. Thus, the delineation of the pathway in Drosophila has helped to identify many genes that function as oncogenes or tumor suppressors in mammals.

The Hippo pathway consists of a core kinase cascade in which Hpo phosphorylates the protein kinase Warts (Wts). Hpo (MST1/2 in mammals) is a member of the Ste-20 family of protein kinases. This highly conserved group of serine/threonine kinases regulates several cellular processes, including cell proliferation, apoptosis, and various stress responses. Once phosphorylated, Wts (LATS1/2 in mammals) becomes active. Misshapen (Msn, MAP4K4/6/7 in mammals) and Happyhour (Hppy, MAP4K1/2/3/5 in mammals) act in parallel to Hpo to activate Wts. Wts is a nuclear DBF-2-related kinase. These kinases are known regulators of cell cycle progression, growth, and development. Two proteins are known to facilitate the activation of Wts: Salvador (Sav) and Mob as tumor suppressor (Mats). Sav (WW45 in mammals) is a WW domain-containing protein, meaning that this protein contains a sequence of amino acids in which a tryptophan and an invariant proline are highly conserved. Hpo can bind to and phosphorylate Sav, which may function as a scaffold protein because this Hpo-Sav interaction promotes phosphorylation of Wts. Hpo can also phosphorylate and activate Mats (MOBKL1A/B in mammals), which allows Mats to associate with and strengthen the kinase activity of Wts. [12]

Components of the Hippo Signaling Pathway include, but are not limited to, DCHS1, DCHS2, FAT1, FAT2, FAT3, FAT4, FRMD6, FERM, WC1, NF2, MST1, MST2, SAV1, LATS1, LATS2, MOBKL1A, MOBKL1B, YAP, TAZ, TEAD1, TEAD2, TEAD3, and TEAD4. Homologs, paralogs, and/or orthologs of components of the Hippo signaling pathway are known in the art and can be readily identified by one skilled in the art. The Hippo Signaling pathway is further reviewed in, e.g., Verghese S, Moberg K. Roles of Membrane and Vesicular Traffic in Regulation of the Hippo Pathway. Front Cell Dev Biol. 2020 Jan 10;7:384. doi: 10.3389/fcell.2019.00384. eCollection 2019; Pocaterra A, Romani P, Dupont S. YAP/TAZ functions and their regulation at a glance. J Cell Sci. 2020 Jan 29;133(2). pii: jcs230425. doi: 10. 1242/jcs.230425; Barzegari A, et al. The role of Hippo signaling pathway and mechanotransduction in tuning embryoid body formation and differentiation. J Cell Physiol. 2020 Jan 17. doi: 10.1002/jcp.29455; Shimoda M, Moroishi T. The Emerging Link between the Hippo Pathway and Non-coding RNA. Biol Pharm Bull. 2020;43(1):1-10. doi: 10.1248/bpb.b19-00795; Vania V, et al. The interplay of signaling pathway in endothelial cells-matrix stiffness dependency with targeted-therapeutic drugs. Biochim Biophys Acta Mol Basis Dis. 2019 Dec 19;1866(5):165645. doi: 10.1016/j.bbadis.2019.165645; Afify AY. A miRNA’s insight into the regenerating heart: a concise descriptive analysis. Heart Fail Rev. 2019 Dec 14. doi: 10.1007/s10741-019-09896-w; Mussell A, Frangou C, Zhang J. Regulation of the Hippo signaling pathway by deubiquitinating enzymes in cancer. Genes Dis. 2019 Jun 24;6(4):335-341. doi: 10.1016/j.gendis.2019.06.004. eCollection 2019 Dec; van Soldt BJ, Cardoso WV. Hippo-Yap/Taz signaling: Complex network interactions and impact in epithelial cell behavior. Wiley Interdiscip Rev Dev Biol. 2019 Dec 11:e371. doi: 10.1002/wdev.371; Ai J, et al. Mesenchymal stromal cells induce inhibitory effects on hepatocellular carcinoma through various signaling pathways. Cancer Cell Int. 2019 Dec 5;19:329. doi: 10.1186/s12935-019-1038-0. eCollection 2019; Zhang C, et al. Regulation of Hippo Signaling by Mechanical Signals and the Cytoskeleton. DNA Cell Biol. 2020 Feb;39(2):159-166. doi: 10.1089/dna.2019.5087. Epub 2019 Dec 10; Santos-de-Frutos K, Segrelles C, Lorz C. Hippo Pathway and YAP Signaling Alterations in Squamous Cancer of the Head and Neck. J Clin Med. 2019 Dec 3;8(12). pii: E2131. doi: 10.3390/jcm8122131; and Rausch V, Hansen CG. The Hippo Pathway, YAP/TAZ, and the Plasma Membrane. Trends Cell Biol. 2020 Jan;30(1):32-48. doi: 10.1016/j.tcb.2019.10.005. Epub 2019 Dec 2. Review; the contents of which are incorporated herein by reference in their entireties.

### Growth-Differentiation Factor 15 (GDF15)

Growth and differentiation factor 15 (GDF15) was first identified as Macrophage inhibitory cytokine-1. GDF15 is a protein belonging to the transforming growth factor beta superfamily. Under normal conditions, GDF-15 is expressed in low concentrations in most organs and upregulated because of injury of organs such as such as liver, kidney, heart and lung. The function of GDF15 appears to be, at least in part, in regulating inflammatory pathways and to be involved in regulating apoptosis, cell repair and cell growth, which are biological processes observed in cardiovascular and neoplastic disorders. GDF15 has shown to be a strong prognostic protein in patients with different diseases such as heart diseases and cancer.

Methods and compositions described herein require that the levels and/or activity of GDF15 are inhibited. As used herein, cytokine growth and differentiation factor 15, also known as “GDF15” refers to a secreted ligand of the TGF-beta (transforming growth factor-beta) superfamily of proteins. GDF15 sequences are known for a number of species, e.g., human GDF15 (NCBI Gene ID: 9518) polypeptide (e.g., NCBI Ref Seq NP_004855.2) and mRNA (e.g., NCBI Ref Seq NM_004864.4). GDF15 can refer to human GDF15, including naturally occurring variants, molecules, and alleles thereof. GDF15 refers to the mammalian GDF15 of, e.g., mouse, rat, rabbit, dog, cat, cow, horse, pig, and the like. The nucleic sequence of SEQ ID NO: 2 comprises a nucleic sequence which encodes GDF15.

SEQ ID NO: 2 contains a nucleic acid sequence that encodes GDF15.

atgcccgg gcaagaactc
aggacggtga
61 atggctctca gatgctcctg gtgttgctgg tgctctcgtg gctgccgcat
gggggcgccc
121 tgtctctggc cgaggcgagc cgcgcaagtt tcccgggacc ctcagagttg
cactccgaag
181 actccagatt ccgagagttg cggaaacgct acgaggacct gctaaccagg
ctgcgggcca
241 accagagctg ggaagattcg aacaccgacc tcgtcccggc ccctgcagtc
cggatactca
301 cgccagaagt gcggctggga tccggcggcc acctgcacct gcgtatctct
cgggccgccc
361 ttcccgaggg gctccccgag gcctcccgcc ttcaccgggc tctgttccgg
ctgtccccga
421 cggcgtcaag gtcgtgggac gtgacacgac cgctgcggcg tcagctcagc
cttgcaagac
481 cccaggcgcc cgcgctgcac ctgcgactgt cgccgccgcc gtcgcagtcg
gaccaactgc
541 tggcagaatc ttcgtccgca cggccccagc tggagttgca cttgcggccg
caagccgcca
601 gggggcgccg cagagcgcgt gcgcgcaacg gggaccactg tccgctcggg
cccgggcgtt
661 gctgccgtct gcacacggtc cgcgcgtcgc tggaagacct gggctgggcc
gattgggtgc
721 tgtcgccacg ggaggtgcaa gtgaccatgt gcatcggcgc gtgcccgagc
cagttccggg
781 cggcaaacat gcacgcgcag atcaagacga gcctgcaccg cctgaagccc
gacacggtgc
841 cagcgccctg ctgcgtgccc gccagctaca atcccatggt gctcattcaa
aagaccgaca
901 ccggggtgtc gctccagacc tatgatgact tgttagccaa agactgccac
tgcatatga
(SEQ ID NO: 2)

### Notch4

The Notch signaling pathway is an evolutionarily conserved intercellular signaling pathway that regulates interactions between physically adjacent cells. Notch signaling regulates multiple cell fate decisions; each Notch family member plays a role in a variety of developmental processes. In mammals, the Notch family is composed of four Notch receptors (Notch1-Notch4) and five ligands [Delta-like ligand 1 (DLL1), DLL3, DLL4, Jagged(Jag)1 and Jag2]. Upon binding to Jagged or Delta-like ligands on an adjacent cell, two sequential proteolytic events release the intracellular domain of Notch (NICD) allowing its translocation to the nucleus. There the NICD converts the DNA binding factor RBP-J from a transcriptional repressor to a transcriptional activator through MAML1-MAML3 binding¹.

The notch protein is cleaved in the trans-Golgi network, and then presented on the cell surface as a heterodimer. The protein functions as a receptor for membrane bound ligands, and may play a role in vascular, renal, and hepatic development.

Methods and compositions described herein require that the levels and/or activity of Notch4 are inhibited. As used herein, Neurogenic locus notch homolog 4, also known as “Notch4” refers to a type I transmembrane protein, which is a member of a family that share structural characteristics, including an extracellular domain consisting of multiple epidermal growth factor-like (EGF) repeats, and an intracellular domain consisting of multiple different domain. Notch4 sequences are known for a number of species, e.g., human Notch4 (NCBI Gene ID: 4855) polypeptide (e.g., NCBI Ref Seq NP_004548.3) and mRNA (e.g., NCBI Ref Seq NM_004557.3). Notch4 can refer to human Notch4, including naturally occurring variants, molecules, and alleles thereof. Notch4 refers to the mammalian Notch4 of, e.g., mouse, rat, rabbit, dog, cat, cow, horse, pig, and the like. The nucleic sequence of SEQ ID NO: 3 comprises a nucleic sequence which encodes Notch4.

SEQ ID NO: 3 contains a nucleic acid sequence that encodes Notch 4.

a tgcagccccc ttcactgctg ctgctgctgc tgctgctgct
gctgctatgt gtctcagtgg tcagacccag agggctgctg tgtgggagtt tcccagaacc
ctgtgccaat ggaggcacct gcctgagcct gtctctggga caagggacct gccagtgtgc
ccctggcttc ctgggtgaga cgtgccagtt tcctgacccc tgccagaacg cccagctctg
ccaaaatgga ggcagctgcc aagccctgct tcccgctccc ctagggctcc ccagctctcc
ctctccattg acacccagct tcttgtgcac ttgcctccct ggcttcactg gtgagagatg
ccaggccaag cttgaagacc cttgtcctcc ctccttctgt tccaaaaggg gccgctgcca
catccaggcc tcgggccgcc cacagtgctc ctgcatgcct ggatggacag gtgagcagtg
ccagcttcgg gacttctgtt cagccaaccc atgtgttaat ggaggggtgt gtctggccac
atacccccag atccagtgcc actgcccacc gggcttcgag ggccatgcct gtgaacgtga
tgtcaacgag tgcttccagg acccaggacc ctgccccaaa ggcacctcct gccataacac
cctgggctcc ttccagtgcc tctgccctgt ggggcaggag ggtccacgtt gtgagctgcg
ggcaggaccc tgccctccta ggggctgttc gaatgggggc acctgccagc tgatgccaga
gaaagactcc acctttcacc tctgcctctg tcccccaggt ttcataggcc cagactgtga
ggtgaatcca gacaactgtg tcagccacca gtgtcagaat gggggcactt gccaggatgg
gctggacacc tacacctgcc tctgcccaga aacctggaca ggctgggact gctccgaaga
tgtggatgag tgtgagaccc agggtccccc tcactgcaga aacgggggca cctgccagaa
ctctgctggt agctttcact gcgtgtgtgt gagtggctgg ggcggcacaa gctgtgagga
gaacctggat gactgtattg ctgccacctg tgccccggga tccacctgca ttgaccgggt
gggctctttc tcctgcctct gcccacctgg acgcacagga ctcctgtgcc acttggaaga
catgtgtctg agccagccgt gccatgggga tgcccaatgc agcaccaacc ccctcacagg
ctccacactc tgcctgtgtc agcctggcta ttcggggccc acctgccacc aggacctgga
cgagtgtctg atggcccagc aaggcccaag tccctgtgaa catggcggtt cctgcctcaa
cactcctggc tccttcaact gcctctgtcc acctggctac acaggctccc gttgtgaggc
tgatcacaat gagtgcctct cccagccctg ccacccagga agcacctgtc tggacctact
tgccaccttc cactgcctct gcccgccagg cttagaaggg cagctctgtg aggtggagac
caacgagtgt gcctcagctc cctgcctgaa ccacgcggat tgccatgacc tgctcaacgg
cttccagtgc atctgcctgc ctggattctc cggcacccga tgtgaggagg atatcgatga
gtgcagaagc tctccctgtg ccaatggtgg gcagtgccag gaccagcctg gagccttcca
ctgcaagtgt ctcccaggct ttgaagggcc acgctgtcaa acagaggtgg atgagtgcct
gagtgaccca tgtcccgttg gagccagctg ccttgatctt ccaggagcct tcttttgcct
ctgcccctct ggtttcacag gccagctctg tgaggttccc ctgtgtgctc ccaacctgtg
ccagcccaag cagatatgta aggaccagaa agacaaggcc aactgcctct gtcctgatgg
aagccctggc tgtgccccac ctgaggacaa ctgcacctgc caccacgggc actgccagag
atcctcatgt gtgtgtgacg tgggttggac ggggccagag tgtgaggcag agctaggggg
ctgcatctct gcaccctgtg cccatggggg gacctgctac ccccagccct ctggctacaa
ctgcacctgc cctacaggct acacaggacc cacctgtagt gaggagatga cagcttgtca
ctcagggcca tgtctcaatg gcggctcctg caaccctagc cctggaggct actactgcac
ctgccctcca agccacacag ggccccagtg ccaaaccagc actgactact gtgtgtctgc
cccgtgcttc aatgggggta cctgtgtgaa caggcctggc accttctcct gcctctgtgc
catgggcttc cagggcccgc gctgtgaggg aaagctccgc cccagctgtg cagacagccc
ctgtaggaat agggcaacct gccaggacag ccctcagggt ccccgctgcc tctgccccac
tggctacacc ggaggcagct gccagactct gatggactta tgtgcccaga agccctgccc
acgcaattcc cactgcctcc agactgggcc ctccttccac tgcttgtgcc tccagggatg
gaccgggcct ctctgcaacc ttccactgtc ctcctgccag aaggctgcac tgagccaagg
catagacgtc tcttcccttt gccacaatgg aggcctctgt gtcgacagcg gcccctccta
tttctgccac tgcccccctg gattccaagg cagcctgtgc caggatcacg tgaacccatg
tgagtccagg ccttgccaga acggggccac ctgcatggcc cagcccagtg ggtatctctg
ccagtgtgcc ccaggctacg atggacagaa ctgctcaaag gaactcgatg cttgtcagtc
ccaaccctgt cacaaccatg gaacctgtac tcccaaacct ggaggattcc actgtgcctg
ccctccaggc tttgtggggc tacgctgtga gggagacgtg gacgagtgtc tggaccagcc
ctgccacccc acaggcactg cagcctgcca ctctctggcc aatgccttct actgccagtg
tctgcctgga cacacaggcc agtggtgtga ggtggagata gacccctgcc acagccaacc
ctgctttcat ggagggacct gtgaggccac agcaggatca cccctgggtt tcatctgcca
ctgccccaag ggttttgaag gccccacctg cagccacagg gccccttcct gcggcttcca
tcactgccac cacggaggcc tgtgtctgcc ctcccctaag ccaggcttcc caccacgctg
tgcctgcctc agtggctatg ggggtcctga ctgcctgacc ccaccagctc ctaaaggctg
tggccctccc tccccatgcc tatacaatgg cagctgctca gagaccacgg gcttgggggg
cccaggcttt cgatgctcct gccctcacag ctctccaggg ccccggtgtc agaaacccgg
agccaagggg tgtgagggca gaagtggaga tggggcctgc gatgctggct gcagtggccc
gggaggaaac tgggatggag gggactgctc tctgggagtc ccagacccct ggaagggctg
cccctcccac tctcggtgct ggcttctctt ccgggacggg cagtgccacc cacagtgtga
ctctgaagag tgtctgtttg atggctacga ctgtgagacc cctccagcct gcactccagc
ctatgaccag tactgccatg atcacttcca caacgggcac tgtgagaaag gctgcaacac
tgcagagtgt ggctgggatg gaggtgactg caggcctgaa gatggggacc cagagtgggg
gccctccctg gccctgctgg tggtactgag ccccccagcc ctagaccagc agctgtttgc
cctggcccgg gtgctgtccc tgactctgag ggtaggactc tgggtaagga aggatcgtga
tggcagggac atggtgtacc cctatcctgg ggcccgggct gaagaaaagc taggaggaac
tcgggacccc acctatcagg agagagcagc ccctcaaacg cagcccctgg gcaaggagac
cgactccctc agtgctgggt ttgtggtggt catgggtgtg gatttgtccc gctgtggccc
tgaccacccg gcatcccgct gtccctggga ccctgggctt ctactccgct tccttgctgc
gatggctgca gtgggagccc tggagcccct gctgcctgga ccactgctgg ctgtccaccc
tcatgcaggg accgcacccc ctgccaacca gcttccctgg cctgtgctgt gctccccagt
ggccggggtg attctcctgg ccctaggggc tcttctcgtc ctccagctca tccggcgtcg
acgccgagag catggagctc tctggctgcc ccctggtttc actcgacggc ctcggactca
gtcagctccc caccgacgcc ggcccccact aggcgaggac agcattggtc tcaaggcact
gaagccaaag gcagaagttg atgaggatgg agttgtgatg tgctcaggcc ctgaggaggg
agaggaggtg ggccaggctg aagaaacagg cccaccctcc acgtgccagc tctggtctct
gagtggtggc tgtggggcgc tccctcaggc agccatgcta actcctcccc aggaatctga
gatggaagcc cctgacctgg acacccgtgg acctgatggg gtgacacccc tgatgtcagc
agtttgctgt ggggaagtac agtccgggac cttccaaggg gcatggttgg gatgtcctga
gccctgggaa cctctgctgg atggaggggc ctgtccccag gctcacaccg tgggcactgg
ggagaccccc ctgcacctgg ctgcccgatt ctcccggcca accgctgccc gccgcctcct
tgaggctgga gccaacccca accagccaga ccgggcaggg cgcacacccc ttcatgctgc
tgtggctgct gatgctcggg aggtctgcca gcttctgctc cgtagcagac aaactgcagt
ggacgctcgc acagaggacg ggaccacacc cttgatgctg gctgccaggc tggcggtgga
agacctggtt gaagaactga ttgcagccca agcagacgtg ggggccagag ataaatgggg
gaaaactgcg ctgcactggg ctgctgccgt gaacaacgcc cgagccgccc gctcgcttct
ccaggccgga gccgataaag atgcccagga caacagggag cagacgccgc tattcctggc
ggcgcgggaa ggagcggtgg aagtagccca gctactgctg gggctggggg cagcccgaga
gctgcgggac caggctgggc tagcgccggc ggacgtcgct caccaacgta accactggga
tctgctgacg ctgctggaag gggctgggcc accagaggcc cgtcacaaag ccacgccggg
ccgcgaggct gggcccttcc cgcgcgcacg gacggtgtca gtaagcgtgc ccccgcatgg
gggcggggct ctgccgcgct gccggacgct gtcagccgga gcaggccctc gtgggggcgg
agcttgtctg caggctcgga cttggtccgt agacttggct gcgcgggggg gcggggccta
ttctcattgc cggagcctct cgggagtagg agcaggagga ggcccgaccc ctcgcggccg
taggttttct gcaggcatgc gcgggcctcg gcccaaccct gcgataatgc gaggaagata
cggagtggct gccgggcgcg gaggcagggt ctcaacggat gactggccct gtgattgggt
ggccctggga gcttgcggtt ctgcctccaa cattccgatc ccgcctcctt g (SEQ ID NO:
3)

### Agents

In one aspect, an agent that inhibits a target described herein (e.g., Wnt signaling, Hippo signaling, GDF15, or Notch4) is administered to a subject having, or at risk of having asthma or an allergic disease. In one embodiment, the agent that inhibits a target is a small molecule, an antibody or antibody fragment, a peptide, an antisense oligonucleotide, a genome editing system, or an RNAi.

An agent is considered effective for inhibiting Wnt signaling if, for example, upon administration, it inhibits the presence, amount, activity and/or level of any component of the Wnt signaling pathway in the cell.

An agent is considered effective for inhibiting Hippo signaling if, for example, upon administration, it inhibits the presence, amount, activity and/or level of any component of the Hippo signaling pathway in the cell.

An agent is considered effective for inhibiting GDF15 if, for example, upon administration, it inhibits the presence, amount, activity and/or level of GDF15 in the cell.

An agent is considered effective for inhibiting Notch4 if, for example, upon administration, it inhibits the presence, amount, activity and/or level of Notch4 in the cell.

In one embodiment, an agent that inhibits Wnt signaling reduces the population of Th2 effector cells. In one embodiment, the population of Th2 effector cells is reduced by at least 10%, by at least 20%, by at least 30%, by at least 40%, by at least 50%, by at least 60%, by at least 70%, by at least 80%, by at least 90%, by at least 99% or more as compared to an appropriate control. As used herein, an “appropriate control” refers to the population of Th2 effector cells prior to administration of the agent or the population of Th2 effector cells in a subject that is not administered the agent. One skilled in the art can determine if a population of Th2 effector cells has been reduced using standard techniques, for example, by identifying the population of Th2 effectors cells using a cell sorting approach, e.g., FACS analysis or flow cytometry, via specific cell surface markers, and quantifying the size of the population, for example, by cell counts or population volume. Human Th2 effector cells can be readily identified, e.g., by the cell combinatorial staining for the surface marker CD4 and intracellular IL-4/IL-13.

In one embodiment, an agent that inhibits Hippo signaling reduces the population of Th17 effector cells. In one embodiment, the population of Th17 effector cells is reduced by at least 10%, by at least 20%, by at least 30%, by at least 40%, by at least 50%, by at least 60%, by at least 70%, by at least 80%, by at least 90%, by at least 99% or more as compared to an appropriate control. As used herein, an “appropriate control” refers to the population of Th17 effector cells prior to administration of the agent or the population of Th17 effector cells in a subject that is not administered the agent. One skilled in the art can determine if a population of Th17 effector cells has been reduced using standard techniques, for example, by identifying the population of Th17 effectors cells using a cell sorting approach, e.g., FACS analysis or flow cytometry, via specific cell surface markers, and quantifying the size of the population, for example, by cell counts or population volume. Th17 effector cells can be readily identified, e.g., by combinatorial staining for the surface marker CD4 and intracellular IL-17.

In one embodiment, an agent that inhibits GDF15 reduces the population of group 2 innate lymphoid cells (ILC2). In one embodiment, the population of ILC2 cells is reduced by at least 10%, by at least 20%, by at least 30%, by at least 40%, by at least 50%, by at least 60%, by at least 70%, by at least 80%, by at least 90%, by at least 99% or more as compared to an appropriate control. As used herein, an “appropriate control” refers to the population of ILC2 cells prior to administration of the agent or the population of ILC2 cells in a subject that is not administered the agent. One skilled in the art can determine if a population of ILC2 cells has been reduced using standard techniques, for example, by identifying the population of ILC2 cells using a cell sorting approach, e.g., FACS analysis or flow cytometry, via specific cell surface markers, and quantifying the size of the population, for example, by cell counts or population volume. ILC2 cells can be readily identified, e.g., by the following cell surface markers: CD3⁻CD4⁻CD19⁻CD11b⁻CD11c-SiglecF⁻T1/ST2+.

In one embodiment, an agent that inhibits Notch4 reduces the population of Notch4-expressing cells, for example, a T reg cell expressing Notch4. In one embodiment, the population of Notch4-expressing cells is reduced by at least 10%, by at least 20%, by at least 30%, by at least 40%, by at least 50%, by at least 60%, by at least 70%, by at least 80%, by at least 90%, by at least 99% or more as compared to an appropriate control. As used herein, an “appropriate control” refers to the population of Notch4-expressing cells prior to administration of the agent or the population of Notch4-expressing cells in a subject that is not administered the agent. One skilled in the art can determine if a population of cells expressing Notch4 has been reduced using standard techniques, for example, biological assays that detect the activity of Notch4 (e.g., Notch reporters that measure the binding of the Notch receptor and ligand) can be used to assess if programmed cell death has occurred. Alternatively, immunofluorescence detection using antibodies specific to Notch4 in combination with cell death markers (e.g., Caspase) can be used to determine if cell death has occurred following administration of an agent.

An agent can inhibit e.g., the transcription, or the translation of a target, e.g., Wnt signaling, Hippo signaling, GDF15, or Notch4, in the cell. In one embodiment, mRNA and protein levels of a given target is reduced by at least 10%, by at least 20%, by at least 30%, by at least 40%, by at least 50%, by at least 60%, by at least 70%, by at least 80%, by at least 90%, by at least 99% or more as compared to an appropriate control. As used herein, an “appropriate control” refers to the mRNA and protein levels of a given target prior to administration of the agent or the mRNA and protein levels of a given target in a cell that is not contacted with the agent. To determine if an agent is effective at inhibiting a target, mRNA and protein levels of the given target (e.g., a Wnt signaling pathway component, a Hippo signaling pathway component, GDF15, or Notch4) can be assessed using RT-PCR and western-blotting, respectively. Any known assays for measure a target’s activity, for example measuring the level of downstream targets of the Wnt or Hippo pathways, can additionally be used.

An agent can inhibit the activity or alter the activity (e.g., such that the activity no longer occurs, occurs at a reduced rate, or occurs in an ineffective manner, e.g., a target is no longer able to activate signaling) of the target in the cell (e.g., the target’s expression). In one embodiment, an agent that inhibits the activity of a target by at least 10%, by at least 20%, by at least 30%, by at least 40%, by at least 50%, by at least 60%, by at least 70%, by at least 80%, by at least 90%, by at least 100% or more as compared to an appropriate control. As used herein, an “appropriate control” refers to the activity of the target prior to administration of the agent, or the activity of target in a population of cells that was not in contact with the agent.

The agent may function directly in the form in which it is administered. Alternatively, the agent can be modified or utilized intracellularly to produce something which inhibits a target, such as introduction of a nucleic acid sequence into the cell and its transcription resulting in the production of the nucleic acid and/or protein inhibitor of the target. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be identified from a library of diverse compounds.

In various embodiments, the agent is a small molecule that inhibits the target. Methods for screening small molecules are known in the art and can be used to identify a small molecule that is efficient at, for example, inducing cell death of pathogenic Th2, Th17, ILC2, or CD4 cells, given the desired target, e.g., Wnt signaling, Hippo signaling, GDF15, and Notch4, respectively.

In various embodiments, agent that inhibits Wnt signaling is any known, or to be discovered, small molecule inhibitor of any component of the Wnt signaling pathway. Exemplary known Wnt signaling inhibitors include, but are not limited to, XAV-939, ICG-001, IWR-1-endo, Wnt-C59 (C59), LGK-974, JW55, ETC-159, iCRT14, KY02111, IWP-2, IWP-L6, Isoquercitrin, PNU-74654, CP21R7 (CP21), Salinomycin (from Streptomyces albus), Adavivint (SM04690), FH535, IWP-01, LF3, WIKI4, Triptonide, PRI-724, GNF-6231, KYA1797K, Methyl Vanillate, iCRT3, WAY-316606, and SKL2001.

In various embodiments, agent that inhibits Hippo signaling is any known, or to be discovered, small molecule inhibitor of any component of the Hippo signaling pathway. Exemplary known Hippo signaling inhibitors include, but are not limited to, (R)-PFI 2 hydrochloride, Verteporfin, YAP inhibitor, XMU MP 1, Ki 16425, and Ro 08-2750.

In various embodiments, the agent that inhibits a target is an antibody or antigen-binding fragment thereof, or an antibody reagent that is specific for the target. As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab’)2, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, nanobodies, humanized antibodies, chimeric antibodies, and the like.

In one embodiment, the agent that inhibits the target is a humanized, monoclonal antibody or antigen-binding fragment thereof, or an antibody reagent. As used herein, “humanized” refers to antibodies from non-human species (e.g., mouse, rat, sheep, etc.) whose protein sequence has been modified such that it increases the similarities to antibody variants produce naturally in humans. In one embodiment, the humanized antibody is a humanized monoclonal antibody. In one embodiment, the humanized antibody is a humanized polyclonal antibody. In one embodiment, the humanized antibody is for therapeutic use.

In one embodiment, the antibody or antibody reagent in an anti-Notch4 antibody or antibody reagent and binds to an amino acid sequence that corresponds to the amino acid sequence encoding Notch4 (SEQ ID NO: 4).

MQPPSLLLLLLLLLLLCVSVVRPRGLLCGSFPEPCANGGTCLSLSLGQGT
CQCAPGFLGETCQFPDPCQNAQLCQNGGSCQALLPAPLGLPSSPSPLTPS
FLCTCLPGFTGERCQAKLEDPCPPSFCSKRGRCHIQASGRPQCSCMPGWT
GEQCQLRDFCSANPCVNGGVCLATYPQIQCHCPPGFEGHACERDVNECFQ
DPGPCPKGTSCHNTLGSFQCLCPVGQEGPRCELRAGPCPPRGCSNGGTCQ
LMPEKDSTFHLCLCPPGFIGPDCEVNPDNCVSHQCQNGGTCQDGLDTYTC
LCPETWTGWDCSEDVDECETQGPPHCRNGGTCQNSAGSFHCVCVSGWGGT
SCEENLDDCIAATCAPGSTCIDRVGSFSCLCPPGRTGLLCHLEDMCLSQP
CHGDAQCSTNPLTGSTLCLCQPGYSGPTCHQDLDECLMAQQGPSPCEHGG
SCLNTPGSFNCLCPPGYTGSRCEADHNECLSQPCHPGSTCLDLLATFHCL
CPPGLEGQLCEVETNECASAPCLNHADCHDLLNGFQCICLPGFSGTRCEE
DIDECRSSPCANGGQCQDQPGAFHCKCLPGFEGPRCQTEVDECLSDPCPV
GASCLDLPGAFFCLCPSGFTGQLCEVPLCAPNLCQPKQICKDQKDKANCL
CPDGSPGCAPPEDNCTCHHGHCQRSSCVCDVGWTGPECEAELGGCISAPC
AHGGTCYPQPSGYNCTCPTGYTGPTCSEEMTACHSGPCLNGGSCNPSPGG
YYCTCPPSHTGPQCQTSTDYCVSAPCFNGGTCVNRPGTFSCLCAMGFQGP
RCEGKLRPSCADSPCRNRATCQDSPQGPRCLCPTGYTGGSCQTLMDLCAQ
KPCPRNSHCLQTGPSFHCLCLQGWTGPLCNLPLSSCQKAALSQGIDVSSL
CHNGGLCVDSGPSYFCHCPPGFQGSLCQDHVNPCESRPCQNGATCMAQPS
GYLCQCAPGYDGQNCSKELDACQSQPCHNHGTCTPKPGGFHCACPPGFVG
LRCEGDVDECLDQPCHPTGTAACHSLANAFYCQCLPGHTGQWCEVEIDPC
HSQPCFHGGTCEATAGSPLGFICHCPKGFEGPTCSHRAPSCGFHHCHHGG
LCLPSPKPGFPPRCACLSGYGGPDCLTPPAPKGCGPPSPCLYNGSCSETT
GLGGPGFRCSCPHSSPGPRCQKPGAKGCEGRSGDGACDAGCSGPGGNWDG
GDCSLGVPDPWKGCPSHSRCWLLFRDGQCHPQCDSEECLFDGYDCETPPA
CTPAYDQYCHDHFHNGHCEKGCNTAECGWDGGDCRPEDGDPEWGPSLALL
WLSPPALDQQLFALARVLSLTLRVGLWVRKDRDGRDMVYPYPGARAEEKL
GGTRDPTYQERAAPQTQPLGKETDSLSAGFVVVMGVDLSRCGPDHPASRC
PWDPGLLLRFLAAMAAVGALEPLLPGPLLAVHPHAGTAPPANQLPWPVLC
SPVAGVILLALGALLVLQLIRRRRREHGALWLPPGFTRRPRTQSAPHRRR
PPLGEDSIGLKALKPKAEVDEDGWMCSGPEEGEEVGQAEETGPPSTCQLW
SLSGGCGALPQAAMLTPPQESEMEAPDLDTRGPDGVTPLMSAVCCGEVQS
GTFQGAWLGCPEPWEPLLDGGACPQAHTVGTGETPLHLAARFSRPTAARR
LLEAGANPNQPDRAGRTPLHAAVAADAREVCQLLLRSRQTAVDARTEDGT
TPLMLAARLAVEDLVEELIAAQADVGARDKWGKTALHWAAAVNNARAARS
LLQAGADKDAQDNREQTPLFLAAREGAVEVAQLLLGLGAARELRDQAGLA
PADVAHQRNHWDLLTLLEGAGPPEARHKATPGREAGPFPRARTVSVSVPP
HGGGALPRCRTLSAGAGPRGGGACLQARTWSVDLAARGGGAYSHCRSLSG
VGAGGGPTPRGRRFSAGMRGPRPNPAIMRGRYGVAAGRGGRVSTDDWPCD
WVALGACGSASNIPIPPPCLTPSPERGSPQLDCGPPALQEMPINQGGEGK
K (SEQ ID NO: 4)

In another embodiment, the anti-Notch4 antibody or antibody reagent binds to an amino acid sequence that comprises the sequence of SEQ ID NO: 4; or binds to an amino acid sequence that comprises a sequence with at least 80%, at least 85%, at least 90%, at least 95%, at least 96%, at least 97%, at least 98%, at least 99% or greater sequence identity to the sequence of SEQ ID NO: 4. In one embodiment, the anti-Notch4 antibody or antibody reagent binds to an amino acid sequence that comprises the entire sequence of SEQ ID NO: 4. In another embodiment, the antibody or antibody reagent binds to an amino acid sequence that comprises a fragment of the sequence of SEQ ID NO: 4, wherein the fragment is sufficient to bind its target, e.g., Notch4, and inhibits the differentiation of a Nocth4-expressing Treg cell into a disease-promoting Th2 cell.

In one embodiment, the agent that inhibits the target (e.g., Wnt signaling, Hippo signaling, GDF15, or Notch4) is an inhibitory peptide. As used herein, an “inhibitory peptide” refers to a fragment polypeptide of a full length gene product, that when expressed in a cell, inhibits, e.g., the function, activity, and/or expression level of the full length gene product. For example, the inhibitory peptide can bind to a target of the full length gene product, preventing activation or silencing of that target by the full length gene product. An inhibitory peptide can comprise at least 5, at least 10, at least 15, at least 20, at least 25, at least 30, or more amino acids that are homologous to a portion of the amino acid sequence of the target.

In one embodiment, the agent is a GDF15 inhibitory peptide and comprises a sequence that is homologous to the amino acid sequence of GDF 15. SEQ ID NO: 5 is the amino acid sequence for GDF15.

1 MPGQELRTVN GSQMLLVLLV LSWLPHGGAL SLAEASRASF PGPSELHSED
SRFRELRKRY
61 EDLLTRLRAN QSWEDSNTDL VPAPAVRILT PEVRLGSGGH LHLRISRAAL
PEGLPEASRL
121 HRALFRLSPT ASRSWDVTRP LRRQLSLARP QAPALHLRLS PPPSQSDQLL
AESSSARPQL
181 ELHLRPQAAR GRRRARARNG DHCPLGPGRC CRLHTVRASL EDLGWADWVL
SPREVQVTMC
241 IGACPSQFRA ANMHAQIKTS LHRLKPDTVP APCCVPASYN PMVLIQKTDT
GVSLQTYDDL
301 LAKDCHCI (SEQ ID NO: 5)

In one embodiment, the GDF15 inhibitory peptide comprises the sequence of KTSLHRLKPDTVPAPC (SEQ ID NO: 1; amino acids 258-273 of the full length GDF15 gene product, SEQ ID NO: 5). In one embodiment, the GDF15 inhibitory peptide consists of, or consists essentially of the sequence of KTSLHRLKPDTVPAPC (SEQ ID NO: 1). In one embodiment, the GDF15 inhibitory peptide comprises a sequence having at least 50%, at least 60%, at least 70%, at least 80%, at least 90%, at least 95%, at least 99% or more sequence identity to (SEQ ID NO: 1), and maintains the same function as wild-type (SEQ ID NO: 1), e.g., inhibits GDF15 function in the cell.

For inhibition of Wnt signaling, the inhibitory peptide that inhibits a target may comprise at least 5, at least 10, at least 15, at least 20, at least 25, at least 30, or more amino acids to a portion of the amino acid sequence of any of the Wnt signaling pathway component genes.

For inhibition of Hippo signaling, the inhibitory peptide that inhibits a target may comprise at least 5, at least 10, at least 15, at least 20, at least 25, at least 30, or more amino acids to a portion of the amino acid sequence of any of the Hippo signaling pathway component genes.

For inhibition of Notch4, the inhibitory peptide that inhibits a target may comprise at least 5, at least 10, at least 15, at least 20, at least 25, at least 30, or more amino acids to a portion of the amino acid sequence of SEQ ID NO: 4.

In one embodiment, the agent that inhibits the target (e.g., Wnt signaling, Hippo signaling, GDF15, or Notch4) is an antisense oligonucleotide. As used herein, an “antisense oligonucleotide” refers to a synthesized nucleic acid sequence that is complementary to a DNA or mRNA sequence, such as that of a microRNA. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under cellular conditions to a gene, e.g., the target gene. Thus, oligonucleotides are chosen that are sufficiently complementary to the target, i.e., that hybridize sufficiently well and with sufficient specificity in the context of the cellular environment, to give the desired effect. For example, an antisense oligonucleotide that inhibits a target may comprise at least 5, at least 10, at least 15, at least 20, at least 25, at least 30, or more bases complementary to a portion of the coding sequence of the target.

For inhibition of Wnt signaling, the antisense oligonucleotide that inhibits a target may comprise at least 5, at least 10, at least 15, at least 20, at least 25, at least 30, or more bases complementary to a portion of the coding sequence of any of the Wnt signaling pathway component genes.

For inhibition of Hippo signaling, the antisense oligonucleotide that inhibits a target may comprise at least 5, at least 10, at least 15, at least 20, at least 25, at least 30, or more bases complementary to a portion of the coding sequence of any of the Hippo signaling pathway component genes.

For inhibition of GDF 15, the antisense oligonucleotide that inhibits a target may comprise at least 5, at least 10, at least 15, at least 20, at least 25, at least 30, or more bases complementary to a portion of the coding sequence the human GDF15 gene (SEQ ID NO: 2).

In the example of Notch4, the antisense oligonucleotide that inhibits a target may comprise at least 5, at least 10, at least 15, at least 20, at least 25, at least 30, or more bases complementary to a portion of the coding sequence the human Notch4 gene (e.g., SEQ ID NO: 3).

In one embodiment, the target is depleted from the cell’s genome using any genome editing system including, but not limited to, zinc finger nucleases, TALENS, meganucleases, and CRISPR/Cas systems. In one embodiment, the genomic editing system used to incorporate the nucleic acid encoding one or more guide RNAs into the cell’s genome is not a CRISPR/Cas system; this can prevent undesirable cell death in cells that retain a small amount of Cas enzyme/protein. It is also contemplated herein that either the Cas enzyme or the sgRNAs are each expressed under the control of a different inducible promoter, thereby allowing temporal expression of each to prevent such interference.

When a nucleic acid encoding one or more sgRNAs and a nucleic acid encoding an RNA-guided endonuclease each need to be administered, the use of an adenovirus associated vector (AAV) is specifically contemplated. Other vectors for simultaneously delivering nucleic acids to both components of the genome editing/fragmentation system (e.g., sgRNAs, RNA-guided endonuclease) include lentiviral vectors, such as Epstein Barr, Human immunodeficiency virus (HIV), and hepatitis B virus (HBV). Each of the components of the RNA-guided genome editing system (e.g., sgRNA and endonuclease) can be delivered in a separate vector as known in the art or as described herein.

In one embodiment, the agent inhibits the target by RNA inhibition. Inhibitors of the expression of a given gene can be an inhibitory nucleic acid. In some embodiments of any of the aspects, the inhibitory nucleic acid is an inhibitory RNA (iRNA). The RNAi can be single stranded or double stranded.

The iRNA can be siRNA, shRNA, endogenous microRNA (miRNA), or artificial miRNA. In one embodiment, an iRNA as described herein effects inhibition of the expression and/or activity of a target, e.g. Wnt signaling, Hippo signaling, GDF15, or Notch4. In some embodiments of any of the aspects, the agent is siRNA that inhibits the target. In some embodiments of any of the aspects, the agent is shRNA that inhibits the target.

One skilled in the art would be able to design siRNA, shRNA, or miRNA for inhibition of a target, e.g., using publically available design tools. siRNA, shRNA, or miRNA is commonly made using companies such as Dharmacon (Layfayette, CO) or Sigma Aldrich (St. Louis, MO).

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions

The RNA of an iRNA can be chemically modified to enhance stability or other beneficial characteristics. The nucleic acids featured in the invention may be synthesized and/or modified by methods well established in the art, such as those described in “Current protocols in nucleic acid chemistry,” Beaucage, S.L. et al. (Edrs.), John Wiley & Sons, Inc., New York, NY, USA, which is hereby incorporated herein by reference.

In one embodiment, the agent is miRNA that inhibits a target. microRNAs are small non-coding RNAs with an average length of 22 nucleotides. These molecules act by binding to complementary sequences within mRNA molecules, usually in the 3’ untranslated (3’UTR) region, thereby promoting target mRNA degradation or inhibited mRNA translation. The interaction between microRNA and mRNAs is mediated by what is known as the “seed sequence”, a 6-8-nucleotide region of the microRNA that directs sequence-specific binding to the mRNA through imperfect Watson-Crick base pairing. More than 900 microRNAs are known to be expressed in mammals. Many of these can be grouped into families on the basis of their seed sequence, thereby identifying a “cluster” of similar microRNAs. A miRNA can be expressed in a cell, e.g., as naked DNA. A miRNA can be encoded by a nucleic acid that is expressed in the cell, e.g., as naked DNA or can be encoded by a nucleic acid that is contained within a vector.

The agent may result in gene silencing of a target gene (e.g., Wnt signaling pathway component gene, Hippo signaling pathway component gene, GDF15 gene, or Notch4 gene), such as with an RNAi molecule (e.g. siRNA or miRNA). This entails a decrease in the mRNA level in a cell for the target by at least about 5%, about 10%, about 20%, about 30%, about 40%, about 50%, about 60%, about 70%, about 80%, about 90%, about 95%, about 99%, about 100% of the mRNA level found in the cell without the presence of the agent. In one preferred embodiment, the mRNA levels are decreased by at least about 70%, about 80%, about 90%, about 95%, about 99%, about 100%. One skilled in the art will be able to readily assess whether the siRNA, shRNA, or miRNA effectively downregulates the target gene, for example by transfecting the siRNA, shRNA, or miRNA into cells and detecting the levels of the mRNA or gene product found within the cell via PCR-based assays or western-blotting, respectively.

The agent may be contained in and thus further include a vector. Many such vectors useful for transferring exogenous genes into target mammalian cells are available. The vectors may be episomal, e.g. plasmids, virus-derived vectors such cytomegalovirus, adenovirus, etc., or may be integrated into the target cell genome, through homologous recombination or random integration, e.g. retrovirus-derived vectors such as MMLV, HIV-1, ALV, etc. In some embodiments, combinations of retroviruses and an appropriate packaging cell line may also find use, where the capsid proteins will be functional for infecting the target cells, e.g., Th2, Th17, ILC2, or Treg cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5’ untranslated (5’UTR) or “leader” sequences and 3’ UTR or “trailer” sequences, as well as intervening sequences (introns) between individual coding segments (exons).

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector.

One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector.

Another non-integrative viral vector is RNA Sendai viral vector, which can produce protein without entering the nucleus of an infected cell. The F-deficient Sendai virus vector remains in the cytoplasm of infected cells for a few passages, but is diluted out quickly and completely lost after several passages (e.g., 10 passages).

Another example of a non-integrative vector is a minicircle vector. Minicircle vectors are circularized vectors in which the plasmid backbone has been released leaving only the eukaryotic promoter and cDNA(s) that are to be expressed.

As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

### Compositions

The agent or composition of agents described herein can be incorporated into compositions or pharmaceutical compositions suitable for administration to a subject for in vivo delivery to cells, tissues, or organs of the subject, or in vitro or ex vivo use thereof.

One aspect herein provides a composition for preventing and/or treating asthma or an allergic disease comprising, consisting of, or consisting essentially of an agent that inhibits Wnt signaling. For example, an agent that inhibits any component of the Wnt signaling pathway.

One aspect herein provides a composition for preventing and/or treating asthma or an allergic disease comprising, consisting of, or consisting essentially of an agent that inhibits Hippo signaling. For example, an agent that inhibits any component of the Hippo signaling pathway.

One aspect herein provides a composition for preventing and/or treating asthma or an allergic disease comprising, consisting of, or consisting essentially of an agent that inhibits GDF15.

In one embodiment, the composition further comprises a Notch4 inhibitor, e.g., an anti-Notch4 antibody.

In one embodiment, the composition comprises, consists of, or consists essentially of a combination, e.g., more than one, of agents that inhibit a target, e.g., Wnt signaling, Hippo signaling, GDF15, and/or Notch4, described herein. Exemplary compositions comprising a combination of agents that inhibit a target are described herein in Table 2. The combinations presented in Table 2 are not meant to be limiting. In Table 2, “X” indicates the given inhibitor is included in the composition.

TABLE 2

Compositions for treatment and/or prevention of asthma or allergic disease comprising, consisting of, or consisting essentially of a combination of agents.

| | | | |
|---|---|---|---|
| Wnt Signaling Inhibitor | Hippo Signaling Inhibitor | GDF15 Inhibitor | Notch4 Inhibitor |
| X | X | | |
| X | | X | |
| X | | | X |
| X | X | X | |
| X | | X | X |
| X | X | | X |
| X | X | X | X |
| | X | X | |
| | X | | X |
| | X | X | X |
| | | X | X |

Typically, a pharmaceutical composition includes the agent or combination of agents described herein and a pharmaceutically acceptable carrier. For example, the agent or combination of agents can be incorporated into a pharmaceutical composition suitable for a desired route of therapeutic administration (e.g., parenteral administration). Passive tissue transduction via high pressure intravenous or intra-arterial infusion, as well as intracellular injection, such as intranuclear microinjection or intracytoplasmic injection, are also contemplated. Pharmaceutical compositions for therapeutic purposes can be formulated as a solution, microemulsion, dispersion, liposomes, or other ordered structure suitable to high viral vector and antibiotic concentration. Sterile injectable solutions can be prepared by incorporating the agent or combination of agents in the required amount in an appropriate buffer with one or a combination of ingredients enumerated above, as required, followed by filtered sterilization. The composition can also include a pharmaceutically acceptable carrier.

Pharmaceutical compositions for therapeutic purposes typically must be sterile and stable under the conditions of manufacture and storage. The composition can be formulated as a solution, microemulsion, dispersion, liposomes, or other ordered structure suitable to high viral vector and antibiotic concentration. Sterile injectable solutions can be prepared by incorporating the viral vector and antibiotic in the required amount in an appropriate buffer with one or a combination of ingredients enumerated above, as required, followed by filtered sterilization.

As used herein, “pharmaceutically acceptable carrier” refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation and is compatible with administration to a subject, for example a human. It must also be suitable for use in contact with any tissues or organs with which it may come in contact, meaning that it must not carry a risk of toxicity, irritation, allergic response, immunogenicity, or any other complication that excessively outweighs its therapeutic benefits. Examples of pharmaceutically acceptable carriers include, but are not limited to, a solvent or dispersing medium containing, for example, water, pH buffered solutions (e.g., phosphate buffered saline (PBS), HEPES, TES, MOPS, etc.), isotonic saline, Ringer’s solution, polyol (for example, glycerol, propylene glycol, liquid polyethylene glycol, and the like), alginic acid, ethyl alcohol, and suitable mixtures thereof. In some embodiments, the pharmaceutically acceptable carrier can be a pH buffered solution (e.g. PBS) or water.

### Administration

In some embodiments, the methods described herein relate to treating a subject having or diagnosed as having an asthma or an allergic disease comprising administering an agent or a combination of agents, or compositions thereof, that inhibits a target described herein, e.g., Wnt signaling, Hippo signaling, or GDF15. Subjects having an asthma or an allergic disease can be identified by a physician using current methods of diagnosing a condition. Symptoms and/or complications of asthma or an allergic disease, which characterize these disease and aid in diagnosis are well known in the art and include but are not limited to, persistent cough, trouble breathing, wheezing, shortness of breath, and skin rash. Tests that may aid in a diagnosis of, e.g. asthma, include but are not limited methacholine challenge, nitric oxide test, allergy testing, and sputum eosinophils. A family history of, e.g., asthma, will also aid in determining if a subject is likely to have the condition or in making a diagnosis of asthma or an allergic disease.

The agents or compositions thereof described herein can be administered to a subject having or diagnosed as having asthma or an allergic disease. In some embodiments, the methods described herein comprise administering an effective amount of an agent(s) to a subject in order to alleviate at least one symptom of, e.g., asthma. As used herein, “alleviating at least one symptom of asthma or an allergic disease” is ameliorating any condition or symptom associated with, e.g., asthma (e.g., persistent cough, trouble breathing, wheezing, shortness of breath, and skin rash). As compared with an equivalent untreated control, such reduction is by at least 5%, 10%, 20%, 40%, 50%, 60%, 80%, 90%, 95%, 99% or more as measured by any standard technique. A variety of means for administering the agents described herein to subjects are known to those of skill in the art. In one embodiment, the agent is administered systemically or locally (e.g., to the lungs). In one embodiment, the agent(s) is administered intravenously. In one embodiment, the agent is administered continuously, in intervals, or sporadically. The route of administration of the agent will be optimized for the type of agent being delivered (e.g., an antibody, a small molecule, an RNAi), and can be determined by a skilled practitioner.

In one embodiment, the agent(s), or composition thereof, is administered through inhalation.

The term “effective amount” as used herein refers to the amount of an agent or combination of agents, or compositions thereof, can be administered to a subject having or diagnosed as having asthma or an allergic disease needed to alleviate at least one or more symptom of, e.g., asthma. The term “therapeutically effective amount” therefore refers to an amount of an agent or combination of agents, or compositions thereof, that is sufficient to provide, e.g., a particular anti-asthma effect when administered to a typical subject. An effective amount as used herein, in various contexts, would also include an amount of an agent sufficient to delay the development of a symptom of, e.g., asthma, alter the course of a symptom of, e.g., asthma (e.g., slowing the progression of loss of lung function, inappropriate breathing, or wheezing), or reverse a symptom of, e.g., (e.g., improve lung function or breathing). Thus, it is not generally practicable to specify an exact “effective amount”. However, for any given case, an appropriate “effective amount” can be determined by one of ordinary skill in the art using only routine experimentation.

In one embodiment, an agent or combination of agents, or compositions thereof, is administered continuously (e.g., at constant levels over a period of time). Continuous administration of an agent can be achieved, e.g., by epidermal patches, continuous release formulations, or on-body injectors.

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the agent, which achieves a half-maximal inhibition of symptoms) as determined in cell culture, or in an appropriate animal model, e.g., an asthmatic mouse model. Levels in plasma can be measured, for example, by high performance liquid chromatography. The effects of any particular dosage can be monitored by a suitable bioassay, e.g., measuring neurological function, or blood work, among others. The dosage can be determined by a physician and adjusted, as necessary, to suit observed effects of the treatment.

### Dosage

“Unit dosage form” as the term is used herein refers to a dosage for suitable one administration. By way of example a unit dosage form can be an amount of therapeutic disposed in a delivery device, e.g., a syringe or intravenous drip bag. In one embodiment, a unit dosage form is administered in a single administration. In another, embodiment more than one unit dosage form can be administered simultaneously.

The dosage of the agent or combination of agents, or compositions thereof, as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

### Combinational Therapy

In one embodiment, an agent or combination of agents, or compositions thereof, described herein is used as a monotherapy. In one embodiment, an agent or combination of agents, or compositions thereof, described herein can be used in combination with other known agents and therapies for asthma or an allergic disease. Administered “in combination,” as used herein, means that two (or more) different treatments are delivered to the subject during the course of the subject’s affliction with the disorder, e.g., the two or more treatments are delivered after the subject has been diagnosed with the disorder or disease (asthma or an allergic disease) and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The agent or combination of agents, or compositions thereof, described herein and the at least one additional therapy can be administered simultaneously, in the same or in separate compositions, or sequentially. For sequential administration, the agent described herein can be administered first, and the additional agent can be administered second, or the order of administration can be reversed. The agent and/or other therapeutic agents, procedures or modalities can be administered during periods of active disorder, or during a period of remission or less active disease. The agent can be administered before another treatment, concurrently with the treatment, posttreatment, or during remission of the disorder.

Exemplary therapeutics used to treat asthma include, but are not limited to, inhaled corticosteroids (e.g., fluticasone (Flonase, Flovent HFA), budesonide (Pulmicort Flexhaler, Rhinocort), flunisolide (Aerospan HFA), ciclesonide (Alvesco, Omnaris, Zetonna), beclomethasone (Qnasl, Qvar), mometasone (Asmanex) and leukotriene modifiers (e.g., montelukast (Singulair), zafirlukast (Accolate) and zileuton (Zyflo)); long-acting beta agonists (e.g., salmeterol (Serevent) and formoterol (Foradil, Perforomist)); combination inhalers (e.g., fluticasone-salmeterol (Advair Diskus), budesonide-formoterol (Symbicort) and formoterol-mometasone (Dulera)); theophylline (e.g., Theophylline (Theo-24, Elixophylline)); short-acting beta agonists (e.g., albuterol (ProAir HFA, Ventolin HFA, others) and levalbuterol (Xopenex)); ipratropium (e.g., Atrovent); and oral and intravenous corticosteroids.

Exemplary therapeutics used to treat an allergic disease include, but are not limited to, anti-inflammatory therapeutics (e.g., corticosteroids, glucocorticoids, or mineralcorticoids); antihistamines (e.g., Brompheniramine (Dimetane), Cetirizine (Zyrtec), Chlorpheniramine (Chlor-Trimeton), Clemastine (Tavist), Diphenhydramine (Benadryl), Fexofenadine (Allegra), or Loratadine (Alavert, Claritin)); and adrenaline.

When administered in combination, the agent or combination of agents, or compositions thereof, and the additional agent (e.g., second or third agent), or all, can be administered in an amount or dose that is higher, lower or the same as the amount or dosage of each agent used individually, e.g., as a monotherapy. In certain embodiments, the administered amount or dosage of the agent, the additional agent (e.g., second or third agent), or all, is lower (e.g., at least 20%, at least 30%, at least 40%, or at least 50%) than the amount or dosage of each agent used individually. In other embodiments, the amount or dosage of agent, the additional agent (e.g., second or third agent), or all, that results in a desired effect (e.g., treatment of asthma or an allergic disease) is lower (e.g., at least 20%, at least 30%, at least 40%, or at least 50% lower) than the amount or dosage of each agent individually required to achieve the same therapeutic effect.

### Parenteral Dosage Forms

Parenteral dosage forms of an agent or combination of agents, or compositions thereof, described herein can be administered to a subject by various routes, including, but not limited to, subcutaneous, intravenous (including bolus injection), intramuscular, and intraarterial. Since administration of parenteral dosage forms typically bypasses the patient’s natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer’s injection, dextrose Injection, dextrose and sodium chloride injection, and lactated Ringer’s injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

### Aerosol Formulations

A composition comprising an agent or combination of agents, or compositions thereof, that inhibits a target (e.g., Wnt signaling, Hippo signaling, or GDF15) described herein can be administered directly to the airways of a subject in the form of an aerosol or by nebulization, e.g., for inhalation. For use as aerosols, an agent that inhibits a target in solution or suspension may be packaged in a pressurized aerosol container together with suitable propellants, for example, hydrocarbon propellants like propane, butane, or isobutane with conventional adjuvants. An agent that inhibits a target can also be administered in a non-pressurized form such as in a nebulizer or atomizer.

The term “nebulization” is well known in the art to include reducing liquid to a fine spray. Preferably, by such nebulization small liquid droplets of uniform size are produced from a larger body of liquid in a controlled manner. Nebulization can be achieved by any suitable means therefore, including by using many nebulizers known and marketed today. For example, an AEROMIST pneumatic nebulizer available from Inhalation Plastic, Inc. of Niles, Ill. When the active ingredients are adapted to be administered, either together or individually, via nebulizer(s) they can be in the form of a nebulized aqueous suspension or solution, with or without a suitable pH or tonicity adjustment, either as a unit dose or multidose device.

As is well known, any suitable gas can be used to apply pressure during the nebulization, with preferred gases to date being those which are chemically inert to a modulator of an agent that inhibits the target. Exemplary gases including, but are not limited to, nitrogen, argon or helium can be used to high advantage.

In some embodiments, an agent or combination of agents, or compositions thereof, that inhibits the target can also be administered directly to the airways in the form of a dry powder. For use as a dry powder, a GHK tripeptide can be administered by use of an inhaler. Exemplary inhalers include metered dose inhalers and dry powdered inhalers.

A metered dose inhaler or “MDI” is a pressure resistant canister or container filled with a product such as a pharmaceutical composition dissolved in a liquefied propellant or micronized particles suspended in a liquefied propellant. The propellants which can be used include chlorofluorocarbons, hydrocarbons or hydrofluoroalkanes. Especially preferred propellants are P134a (tetrafluoroethane) and P227 (heptafluoropropane) each of which may be used alone or in combination. They are optionally used in combination with one or more other propellants and/or one or more surfactants and/or one or more other excipients, for example ethanol, a lubricant, an anti- oxidant and/or a stabilizing agent. The correct dosage of the composition is delivered to the patient.

A dry powder inhaler (i.e. Turbuhaler (Astra AB)) is a system operable with a source of pressurized air to produce dry powder particles of a pharmaceutical composition that is compacted into a very small volume.

Dry powder aerosols for inhalation therapy are generally produced with mean diameters primarily in the range of <5 µm. As the diameter of particles exceeds 3 µm, there is increasingly less phagocytosis by macrophages. However, increasing the particle size also has been found to minimize the probability of particles (possessing standard mass density) entering the airways and acini due to excessive deposition in the oropharyngeal or nasal regions.

Suitable powder compositions include, by way of illustration, powdered preparations of an agent or combination of agents, or compositions thereof, that inhibits the target thoroughly intermixed with lactose, or other inert powders acceptable for intrabronchial administration. The powder compositions can be administered via an aerosol dispenser or encased in a breakable capsule which may be inserted by the patient into a device that punctures the capsule and blows the powder out in a steady stream suitable for inhalation. The compositions can include propellants, surfactants, and co-solvents and may be filled into conventional aerosol containers that are closed by a suitable metering valve.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic an diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S. and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

### Controlled and Delayed Release Dosage Forms

In some embodiments of the aspects described herein, an agent or combination of agents, or compositions thereof, is administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control a compound of formula (I)’s onset of action, duration of action, plasma levels within the therapeutic window, and peak blood levels. In particular, controlled- or extended-release dosage forms or formulations can be used to ensure that the maximum effectiveness of an agent is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with any an agent or combination of agents, or compositions thereof, described herein. Examples include, but are not limited to, those described in U.S. Pat. Nos.: 3,845,770; 3,916,899; 3,536,809; 3,598,123; 4,008,719; 5674,533; 5,059,595; 5,591,767; 5,120,548; 5,073,543; 5,639,476; 5,354,556; 5,733,566; and 6,365,185, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, DUOLITE® A568 and DUOLITE® AP143 (Rohm&Haas, Spring House, Pa. USA).

### Efficacy

The efficacy of an agent or combination of agents, or compositions thereof, described herein, e.g., for the treatment and/or prevention of an asthma or an allergic disease, can be determined by the skilled practitioner. However, a treatment is considered “effective treatment,” as the term is used herein, if one or more of the signs or symptoms of, e.g., asthma, are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g., decreased airway inflammation, increased lung function, restored normal breathing. Efficacy can also be measured by a failure of an individual to worsen as assessed by hospitalization, or need for medical interventions (i.e., progression of diminished lung function, complications with breathing, asthmatic attack frequencies). Methods of measuring these indicators are known to those of skill in the art and/or are described herein.

Efficacy can be assessed in animal models of a condition described herein, for example, a mouse model or an appropriate animal model of asthma or allergic disease, as the case may be. When using an experimental animal model, efficacy of treatment is evidenced when a statistically significant change in a marker is observed, e.g., decreased airway inflammation, increased lung function, restored normal breathing.

Efficacy of an agent can additionally be assessed using methods described herein.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway’s Immunobiology, Kenneth Murphy, Allan Mowat, Casey Weaver (eds.), W. W. Norton & Company, 2016 (ISBN 0815345054, 978-0815345053); Lewin’s Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

The invention can further be described in the following numbered paragraphs:
1) A method for treating asthma or an allergic disease, comprising administering to a subject having asthma or an allergic disease an effective amount of an agent that inhibits Wnt signaling.
2) A method for treating asthma or an allergic disease, comprising administering to a subject having asthma or an allergic disease an effective amount of an agent that inhibits Hippo signaling.
3) A method for treating asthma or an allergic disease, comprising administering to a subject having asthma or an allergic disease an effective amount of an inhibitor of Growth-differentiation factor 15 (GDF15).
4) The method of any preceding paragraph, further comprising administering an agent that inhibits Growth/differentiation factor 15 (GDF15).
5) The method of any preceding paragraph, further comprising administering an agent that inhibits Notch4.
6) The method of any preceding paragraph, further comprising administering an agent that inhibits Wnt signaling and an agent that inhibits Hippo signaling.
7) The method of any preceding paragraph, further comprising, prior to administering, diagnosing a subject as having asthma or an allergic disease.
8) The method of any preceding paragraph, further comprising, prior to administering, receiving the results of an assay that diagnoses a subject as having asthma or an allergic disease.
9) The methods of any preceding paragraph, wherein the asthma is selected from the list consisting of allergic asthma, asthma without allergies, aspirin exacerbated respiratory disease, exercise induced asthma, cough variant, and occupational asthma.
10) The methods of any preceding paragraph, wherein the allergic disease is selected from the list consisting of allergic rhinitis, sinusitis, otitis media, atopic dermatitis, urticaria, angioedema, and anaphylaxis.
11) The method of any preceding paragraph, wherein the agent is selected from the group consisting of a small molecule, an antibody, a peptide, a genome editing system, an antisense oligonucleotide, and an RNAi.
12) The method of any preceding paragraph, wherein the antibody is a humanized antibody.
13) The method of any preceding paragraph, wherein the RNAi is a microRNA, an siRNA, or a shRNA.
14) The method of any preceding paragraph, wherein the small molecule is an inhibitor of Wnt signaling, and is selected from the group consisting of XAV-939, ICG-001, IWR-1-endo, Wnt-C59 (C59), LGK-974, JW55, ETC-159, iCRT14, KY02111, IWP-2, IWP-L6, Isoquercitrin, PNU-74654, CP21R7 (CP21), Salinomycin (from Streptomyces albus), Adavivint (SM04690), FH535, IWP-O1, LF3, WIKI4, Triptonide, PRI-724, GNF-6231, KYA1797K, Methyl Vanillate, iCRT3, WAY-316606, and SKL2001.
15) The method of any preceding paragraph, wherein the small molecule is an inhibitor of Hippo signaling, and is selected from the group consisting of (R)-PFI 2 hydrochloride, Verteporfin, YAP inhibitor, XMU MP 1, Ki 16425, and Ro 08-2750.
16) The method of any preceding paragraph, wherein the peptide is an inhibitor of GDF15, and has a sequence of (aa258-273).
17) The method of any preceding paragraph, wherein the antibody is an anti-Notch4 antibody.
18) The method of any preceding paragraph, wherein inhibiting GDF15 is inhibiting GFD15 expression level or activity.
19) The method of any preceding paragraph, wherein inhibiting Wnt signaling reduces the population of Th2 effector cells.
20) The method of any preceding paragraph, wherein inhibiting Hippo signaling reduces the population of Th17 effector cells
21) The method of any preceding paragraph, wherein inhibiting GDF15 reduces the population of group 2 innate lymphoid cell (II,C2).
22) The method of any preceding paragraph, wherein the population is reduced at least 50%, 60%, 70%, 80%, 90%, 95%, or more are compared to an appropriate control.
23) The method of any preceding paragraph, further comprising administering at least one additional anti-asthma therapeutic.
24) The method of any preceding paragraph, further comprising administering at least one additional anti-allergic disease therapeutic.
25) A method for preventing asthma or an allergic disease, comprising administering to a subject at risk of having asthma or an allergic disease an effective amount of an agent that inhibits Wnt signaling.
26) A method for preventing asthma or an allergic disease, comprising administering to a subject at risk of having asthma or an allergic disease an effective amount of an agent that inhibits Hippo signaling.
27) A method for preventing asthma or an allergic disease, comprising administering to a subject at risk of having asthma or an allergic disease an effective amount of an inhibitor of Growth-differentiation factor 15 (GDF15).
28) The method of any preceding paragraph, further comprising administering an agent that inhibits GDF15.
29) The method of any preceding paragraph, further comprising administering an agent that inhibits Notch4.
30) The method of any preceding paragraph, further comprising administering an agent that inhibits Wnt signaling and an agent that inhibits Hippo signaling.
31) The method of any preceding paragraph, further comprising, prior to administering, diagnosing a subject as being at risk of having asthma or an allergic disease.
32) The method of any preceding paragraph, further comprising, prior to administering, receiving the results of an assay that diagnoses a subject as being at risk of having asthma or an allergic disease.
33) A composition for preventing or treating asthma or an allergic disease, comprising an agent that inhibits Wnt signaling and a pharmaceutically acceptable carrier.
34) A composition for preventing or treating asthma or an allergic disease, comprising an agent that inhibits Hippo signaling and a pharmaceutically acceptable carri er.
35) A composition for preventing or treating asthma or an allergic disease, comprising an agent that inhibits GDF15 and a pharmaceutically acceptable carrier.
36) The composition of any preceding paragraph, further comprising an agent that inhibits GDF15.
37) The composition of any preceding paragraph, further comprising an agent that inhibits Notch4.
38) The composition of any preceding paragraph, further comprising an agent that inhibits Wnt signaling and an agent that inhibits Hippo signaling.
39) An agent that inhibits the Wnt signaling pathway.
40) An agent that inhibits the Hippo signaling pathway.
41) An agent that inhibits GDF15.
42) An agent that inhibits Notch4.
43) A method for treating asthma or an allergic disease, the method comprising:
   a. obtaining a biological sample from a subject;
   b. measuring the level of Notch4 in the biological sample of (a);
   c. comparing the level of (b) with a reference level, wherein a subject is identified as having asthma or an allergic disease if the level of (b) is greater than a reference level; and
   d. administering to the subject identified as having at risk asthma or an allergic disease any of the compositions of any preceding paragraph, or agents of any preceding paragraph.
44) A method for preventing asthma or an allergic disease, the method comprising:
   a. obtaining a biological sample from a subject;
   b. measuring the level of Notch4 in the biological sample of (a);
   c. comparing the level of (b) with a reference level, wherein a subject is identified as being at risk of having asthma or an allergic disease if the level of (b) is greater than a reference level; and
   d. administering to the subject identified as having at risk asthma or an allergic disease any of the compositions of any preceding paragraph, or agents of any preceding paragraph.

### EXAMPLES

A Hallmark of asthma is a chronic inflammatory process that is associated with airway hyper-responsiveness and tissue remodeling (1, 2). The persistence of asthmatic inflammation in the face of countervailing immunoregulatory mechanisms that normally limit tissue damage suggests that the latter may become compromised (3). In agreement with this premise, subversion of allergen-specific FOXP3⁺ regulatory T (T_{reg}) cells, leading to the loss of their immune regulatory activity and their conversion in to T helper type 2 and type 17 (Th2/Th17) T effector (T_{eff})-like cells, has emerged as a key pathogenic mechanism (3-6). Elucidating the molecular mechanisms of T_{reg} cell subversion in asthma and means of restoring their function would offer novel approaches to therapy.

Relevant to immune tolerance breakdown in allergic airway inflammation are the inventors’ recent studies on mechanisms by which air polluting ambient particulate matter (PM), and especially ultrafine particles (UFP), upregulate allergic airway inflammation (7, 8). These particles are taken up by alveolar macrophages, where they activate the aryl hydrocarbon receptor to induce the expression of the Notch ligand Jagged1 (Jag1). In turn, Jag1 engages Notch receptors on CD4⁺ T cells to promote mixed T helper 2 (Th2) and Th17 cell-dependent inflammation. Surprisingly, functional antibody inhibition studies pointed to Notch4 as the Notch receptor involved in this pathway (8). The identity of the CD4⁺ T cell subpopulation(s) expressing Notch4, the signals that regulate its induction and its downstream effector pathways remained unknown. Here, the inventors identified Notch4 as a master molecular switch that subverts lung tissue T_{reg} cell function to promote allergic airway inflammation. Notch4 is mainly induced on allergen-specific induced (i)T_{reg} cells in an allergen and interleukin-6 (IL-6)-dependent manner, and acts to disrupt their function by Wnt and Hippo pathway-dependent mechanisms. Importantly, Notch4 acts via the Wnt pathway to induce the expression in lung tissue T_{reg} cells of the cytokine Growth and differentiation factor 15 (GDF15), a cytokine previously implicated in metabolic adaption to inflammation (9, 10), which the inventors have shown herein to upregulate allergic tissue inflammation by directly activating group 2 innate lymphoid cells (ILC2). These findings place Notch4 at the intersection of allergen and pollutant-driven airway inflammation and indicate a novel intervention strategies targeting Notch4 to restore long-term immune tolerance in asthma and related disorders.

### Notch4 is Inducibly Expressed on T_{reg} Cells in Allergic Airway Inflammation

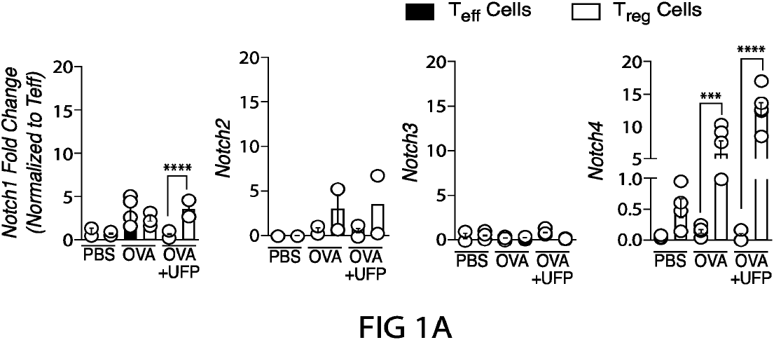

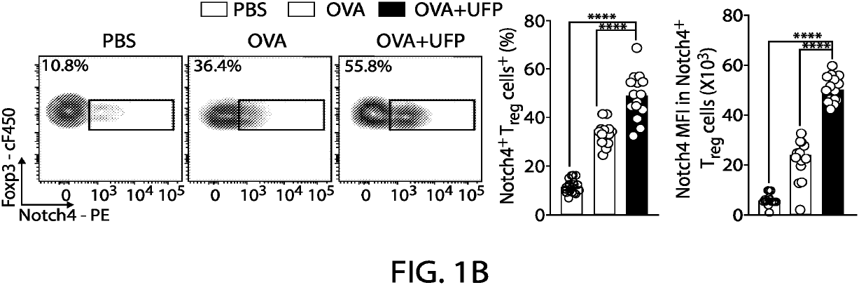

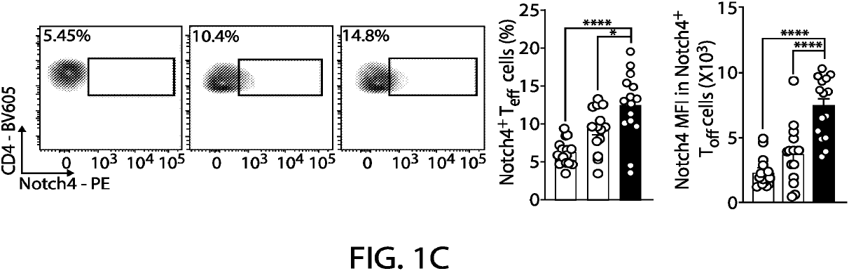

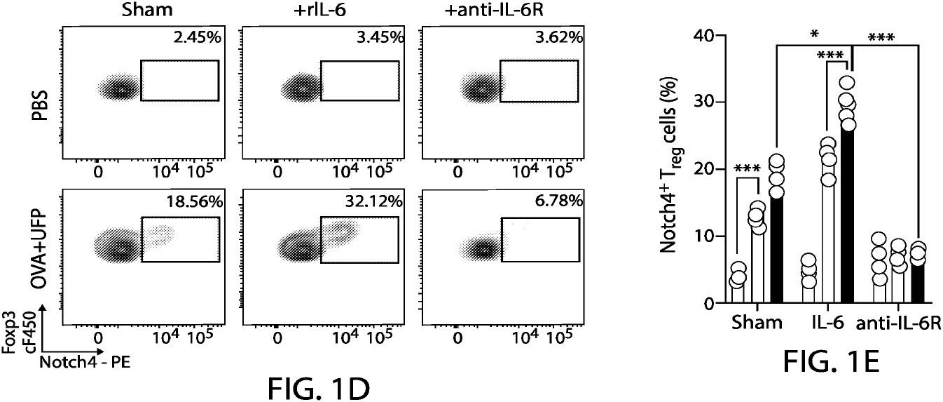

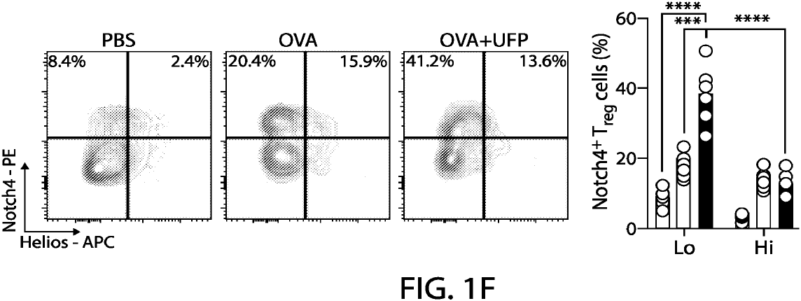

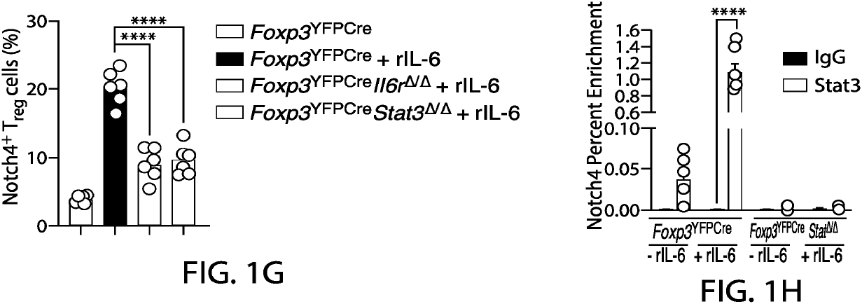

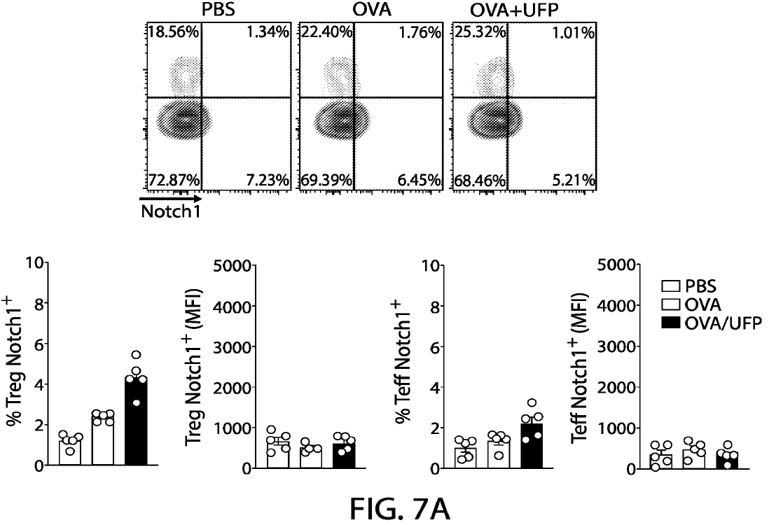

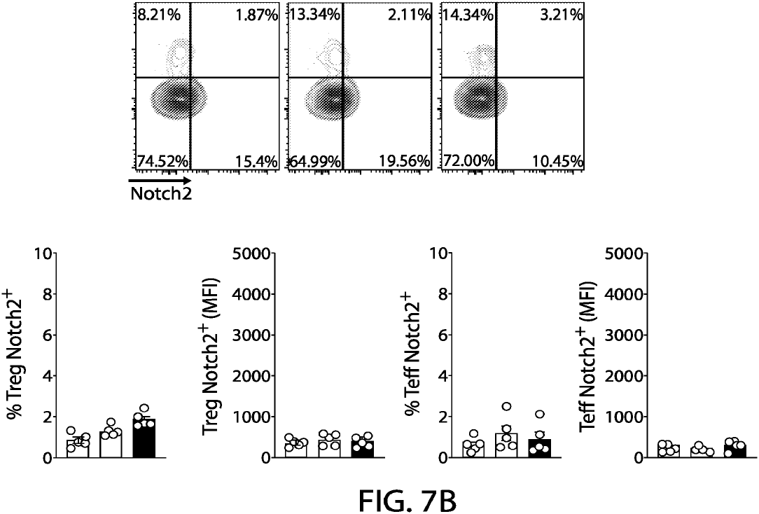

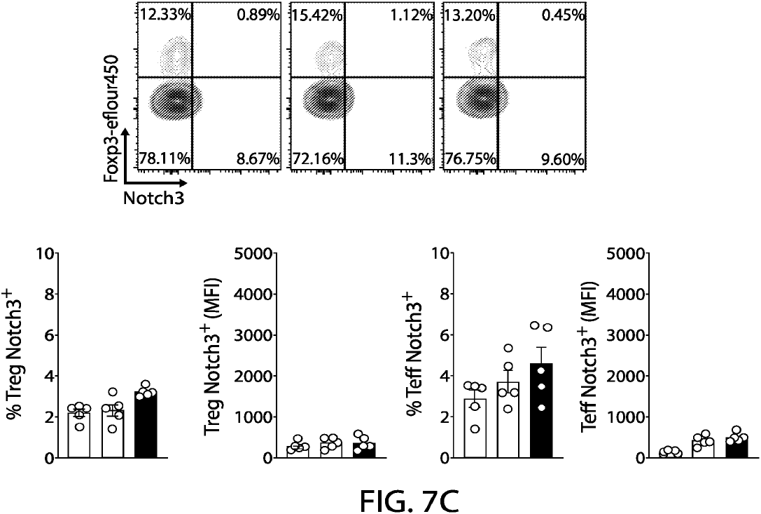

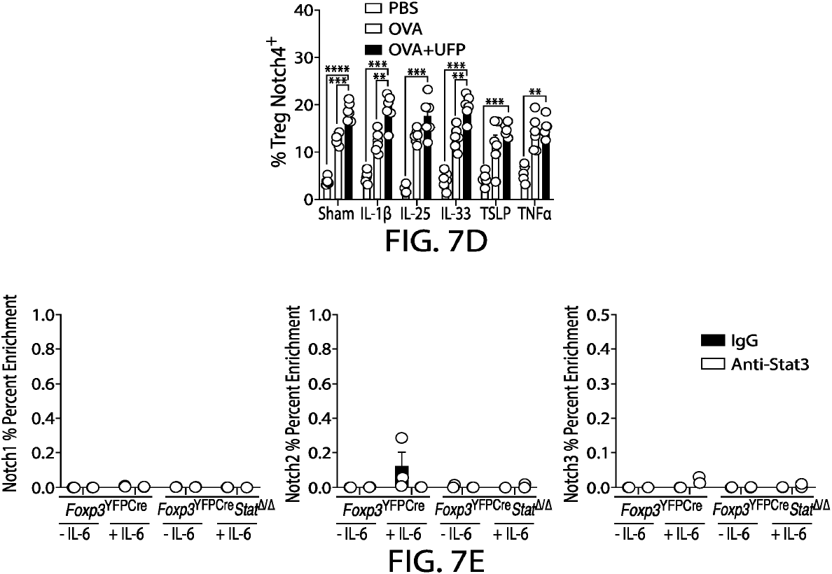

The inventors determined by real time (RT-)PCR the identity of the Notch receptor species expressed in lung tissue T_{reg} and T_{eff} cells isolated from sham and ovalbumin (OVA)-sensitized mice following their challenge with OVA, and from OVA-sensitized mice co-treated with intranasal UFP during the OVA challenge phase. Results showed that Notch4 expression was enriched in lung T_{reg} cells at baseline and was sharply upregulated in OVA and especially OVA+UFP treated mice relative to other Notch receptor species (FIG. 1A). These results were supported by flow cytometric analysis of the expression of respective Notch receptors in T_{reg} and T_{eff} cells, which confirmed the differential upregulation of Notch4 on lung T_{reg} cells in allergic airway inflammation (FIGS. 1B and C; FIG. 7A). Notch4 localized differentially to Helios⁻ T_{reg} cells, consistent with its enrichment on iT_{reg} cells (FIG. 1F).

To examine signals driving the induction of Notch4 on iT_{reg} cells, the inventors employed an in vitro iT_{reg} cell differentiation system in which OT-II T cell receptor (TCR) transgenic T cells, specific for the OVA₃₂₃-₃₃₉ peptide, were incubated with isolated primary alveolar macrophages (AM). The latter cell type potently drives iT_{reg} cell differentiation under non-inflammatory conditions, and at the same time critically promotes allergic airway inflammation by allergens and UFP by virtue of their inducible expression of Notch ligands, most notably Jagged 1 (Jag1) (8). Results revealed a step-wise increase in Notch4 expression in differentiating iT_{reg} cells in co-cultures with OVA₃₂₃-₃₃₉- and OVA₃₂₃-₃₃₉ +UFP-pulsed AM (FIGS. 1D and 1E). Addition of IL-6 to the cell cultures, but not IL-1β, TNFa, IL-25 and TSLP, resulted in super-induction of Notch4 expression on differentiating iT_{reg} cells in an OVA₃₂₃-₃₃₉ antigen-dependent manner. IL-33 treatment did not induce Notch4 on its own, but further upregulated the expression of Notch4 induced by IL-6 (FIGS. 1D and E; FIGS. 7B and 7C). In contrast, treatment with an anti-IL-6 receptor (IL-6R) mAb suppressed Notch4 expression induced by OVA₃₂₃-₃₃₉- and OVA₃₂₃-₃₃₉ +UFP-pulsed AM. Furthermore, in an antigen presenting cell-free system of in vitro iT_{reg} cell differentiation by treatment of naive T cells with anti-CD3+anti-CD28 mAbs in the presence of transforming growth factor beta 1 (TGF-β1) (11), addition of IL-6 to the cell culture induced Notch4 on differentiating T_{reg} cells, which was attenuated by T_{reg} cell-specific deletion of IL-6R alpha chain or the downstream transcription factor STAT3 using a Foxp3-driven Cre recombinase and floxed target alleles (Foxp3^{YFPCre}Il6ra^{Δ/Δ} and Foxp3^{YFPCre}Stat3^{Δ/Δ}, respectively) (FIG. 1H). Chromatin immunoprecipitation assays confirmed IL-6-dependent inducible binding of STAT3 to the Notch4 promoter in T_{reg} cells, but not to those of Notch1, Notch2 or Notch3, which was abrogated by Stat3 deletion (FIG. 1I; FIG. 7D). These results identified IL-6 is a key inducer of Notch4 expression on differentiating allergen-specific lung tissue iT_{reg} cells.

### Notch4 Subverts T_{reg} Cell-Mediated Immune Tolerance in Allergic Airway Inflammation

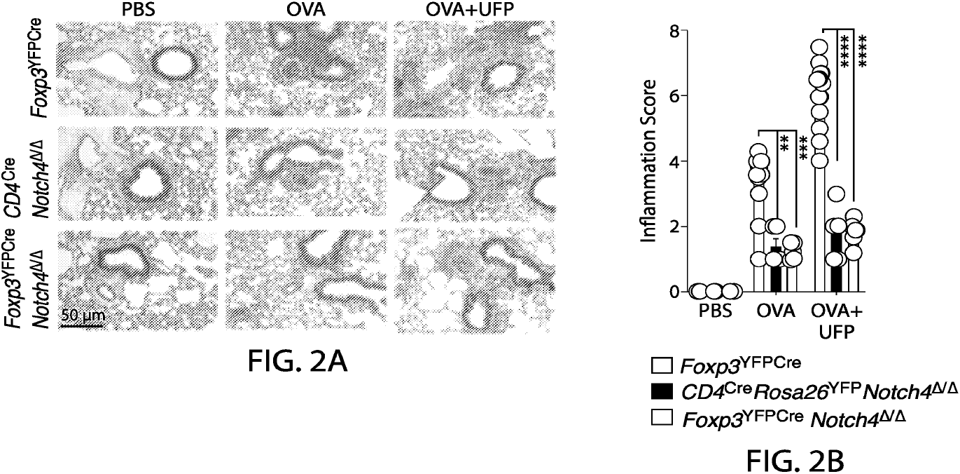

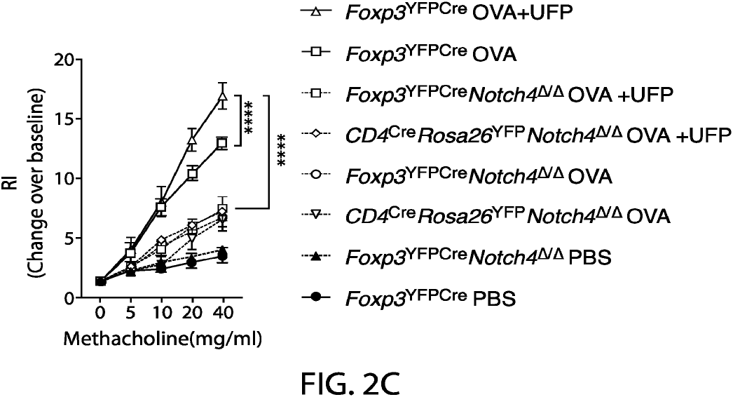

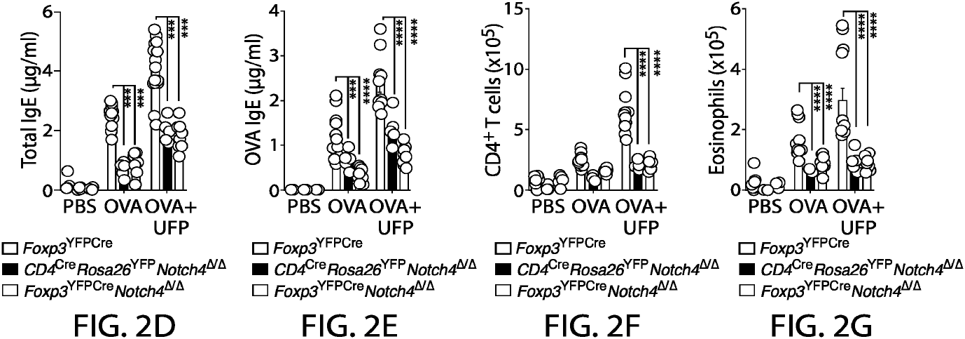

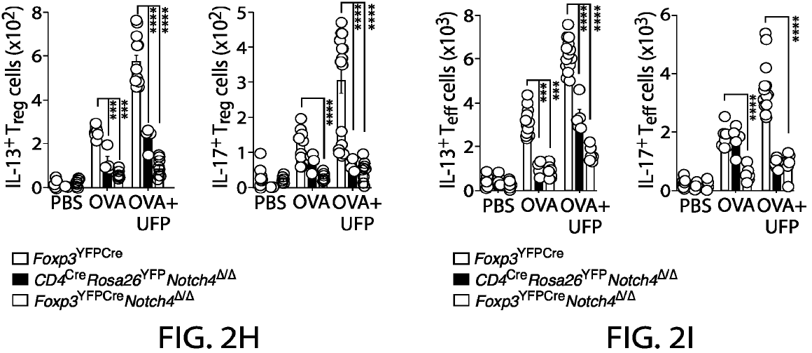

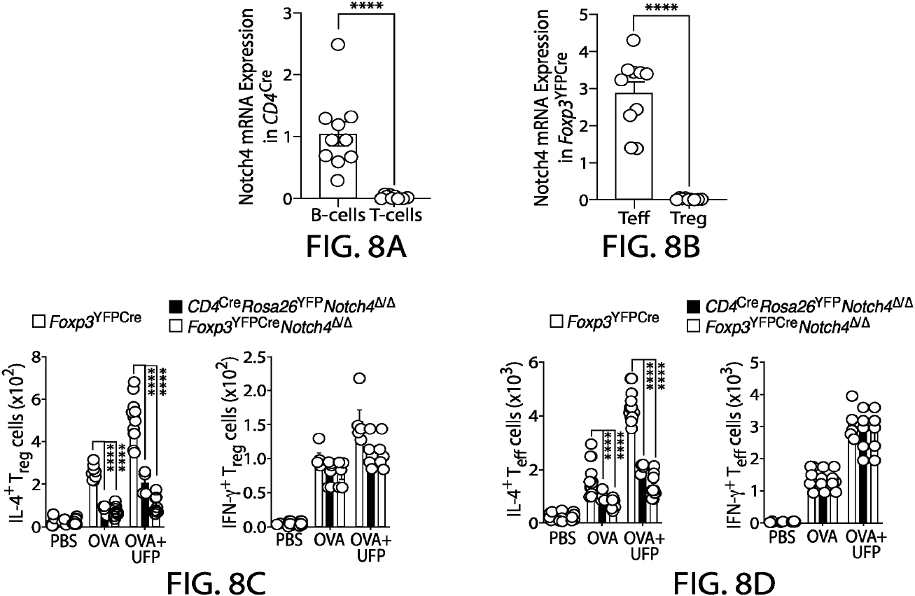

To elucidate the role of inducible Notch4 expression on lung allergen-specific iT_{reg} cells in allergic airway inflammation, the inventors employed CD4^{Cre}Notch4^{Δ/Δ} mice, in which a floxed Notch4 allele is specifically deleted in all T cells (FIGS. 8A-8D). Results showed that deletion of Notch4 in T cells greatly attenuated airway inflammation induced in OVA-sensitized and challenged mice, without or with UFP treatment (FIGS. 2A and 2B). Notch4 deletion largely suppressed the increase in airway hyper-responsiveness induced by OVA, and its super induction by UFP co-treatment (FIG. 2C). To dissect the contribution of Notch4 expression on iT_{reg} cells to the inflammatory response, the inventors employed Foxp3^{YFPCre}Notch4^{Δ/Δ} mice, in which the floxed Notch4 allele is specifically deleted in T_{reg} but not T_{eff} cells (FIGS. 8A-8D). The attenuated allergic airway inflammation noted in CD4^{Cre}Notch4^{Δ/Δ} mice was completely reproduced in Foxp3^{YFPCre}Notch4^{Δ/Δ} mice, indicating that the effect of Notch4 deletion localized to T_{reg} but not T_{eff} cells (FIGS. 2A to 2C). Notch4 deletion in CD4⁺ T cells or in specifically T_{reg} cells suppressed the total and OVA-specific IgE responses, and T cell and eosinophil infiltration of lung tissues (FIGS. 2D to G). It suppressed the lung tissue Th2 and Th17 cell responses, and reversed the destabilization of lung tissue T_{reg} cells towards Th2 and Th17 cell-like phenotypes, while keeping the T_{eff} and T_{reg} cell IFN_{Y} response unaltered (FIGS. 2H and I; FIGS. 8A-8D).

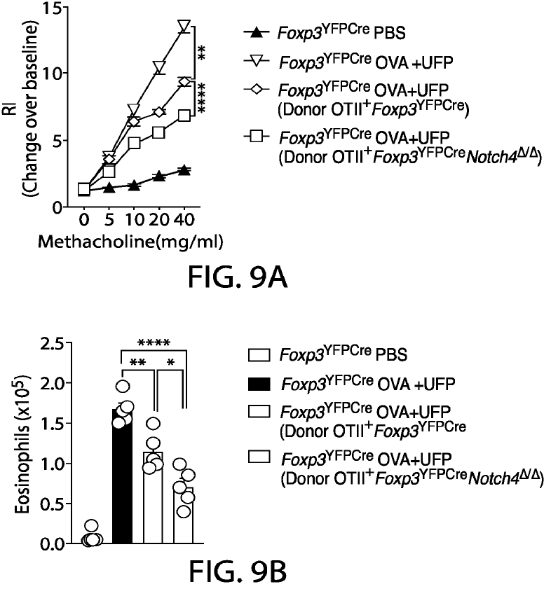

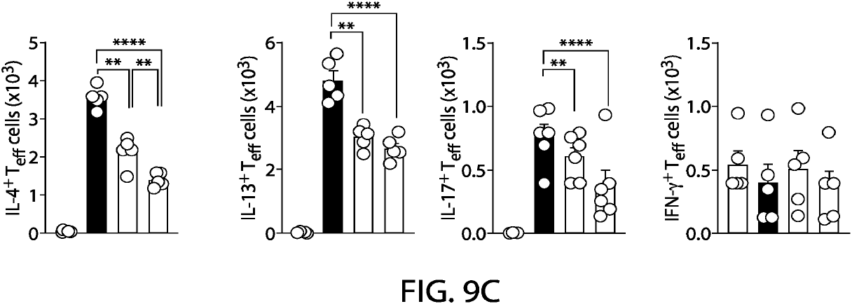

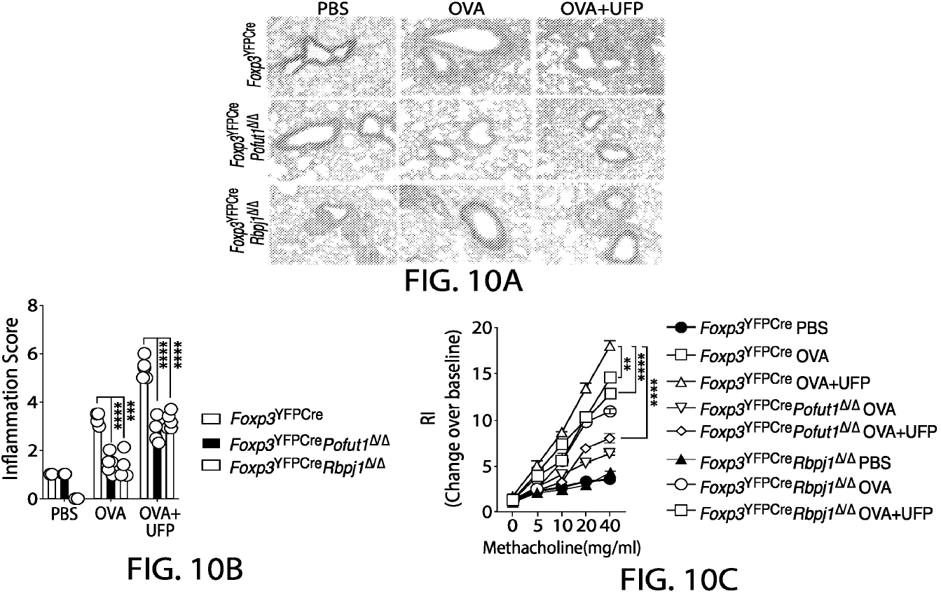

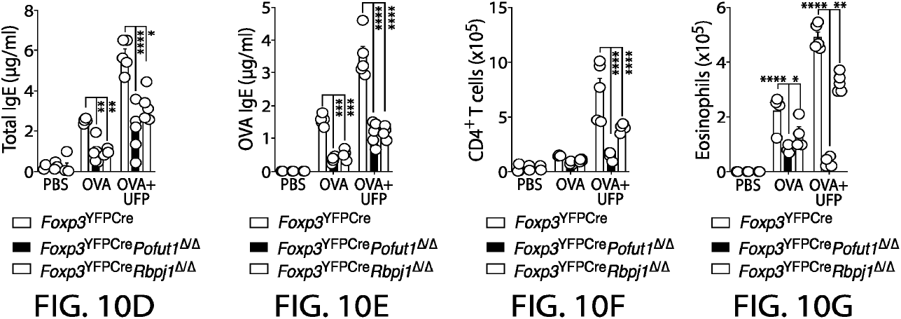

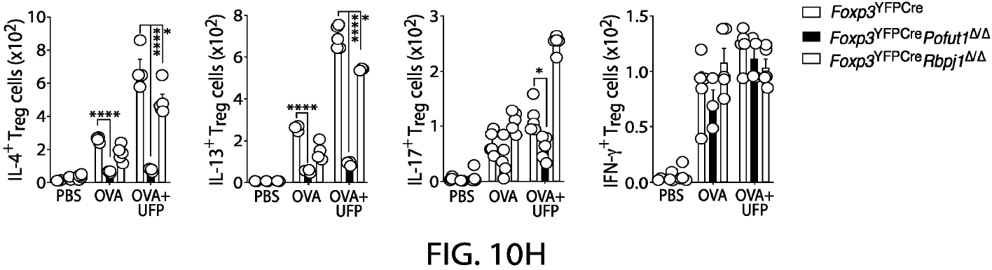

The salutary effects of Notch4 deletion on allergic airway inflammation was fully reproduced by T_{reg} cell-specific deletion of Pofutl, encoding an enzyme that mediates o-fucosylation of Notch receptors, a requisite event in their glycosylation and essential to their function (FIGS. 9A-9C) (12, 13). To determine the role of the canonical versus non-canonical pathways in mediating the effects of Notch signaling on T_{reg} cells in airway inflammation, the inventors examined the impact of deleting Rbpj, encoding the canonical Notch factor RBPJ, in T_{reg} cells on airway responses (13, 14). Results revealed that mice with T_{reg} cell specific deletion of Rpbj (Foxp3^{YFPCre}Rpbj^{Δ/Δ}) exhibited an intermediate phenotype of decreased airway hyperresponsiveness and tissue eosinophilia in-between those of Foxp3^{YFPCre}Pofutl^{Δ/Δ} and Foxp3^{YFPCre} mice (FIGS. 9A-9C). In contrast, T_{reg} cell-specific deletion of floxed Notchl or Notch2 alleles, or global deletion of Notch3, had no impact on allergic airway inflammation (FIGS. 10A-10I).

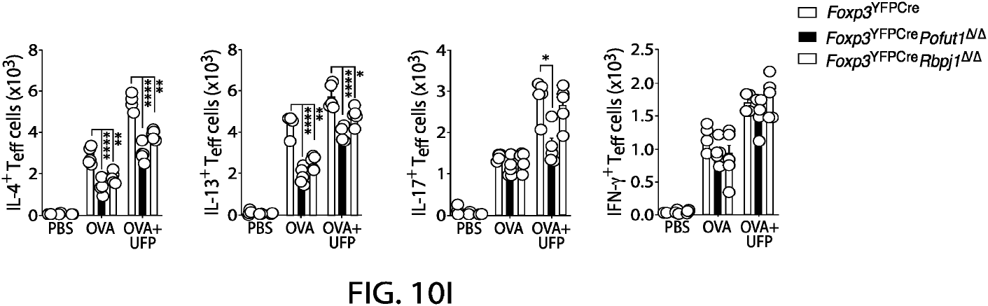

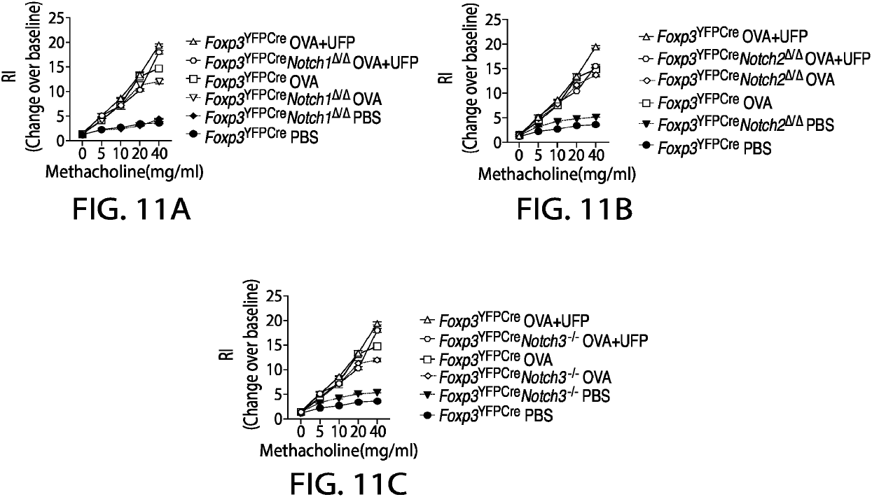

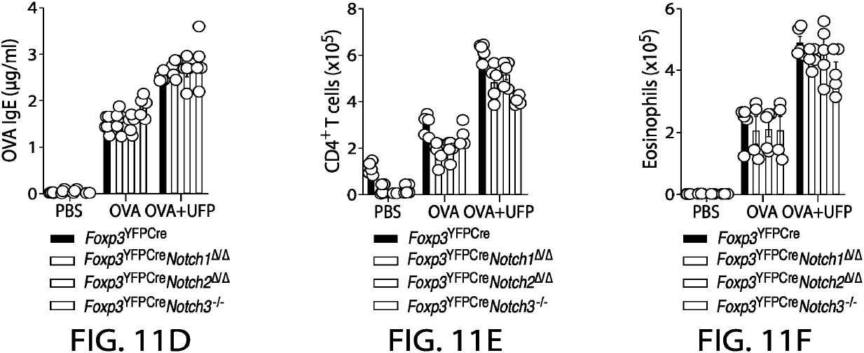

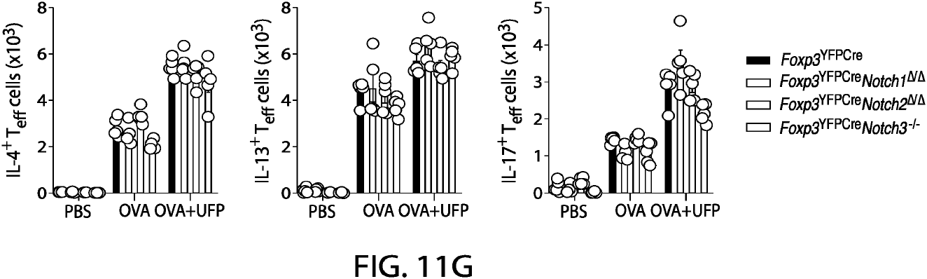

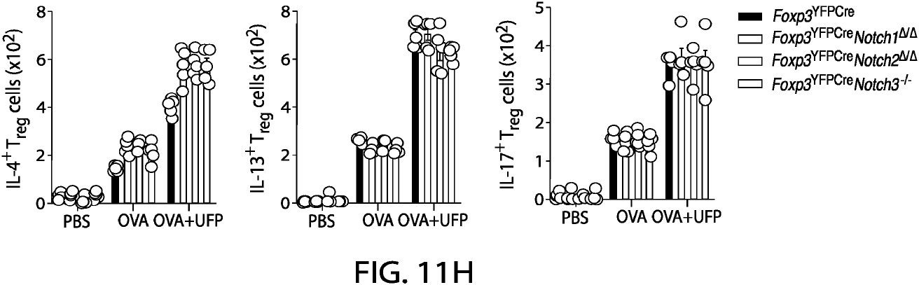

The relationship between Notch4 expression in T_{reg} cells and airway inflammation was also investigated by interrupting upstream pathways regulating its expression. T_{reg} cell-specific deletion of Il6ra or Stat3 recapitulated the protective effect of T_{reg} cell Notch4 deficiency. Both deletions attenuated OVA-induced allergic airway inflammation, with decreased AHR, airway eosinophilia, total and OVA specific IgE, and T_{H}2 and T_{H}17 cell responses, in association with markedly decreased Notch4 expression on T_{reg} cells (FIGS. 11A-11H).

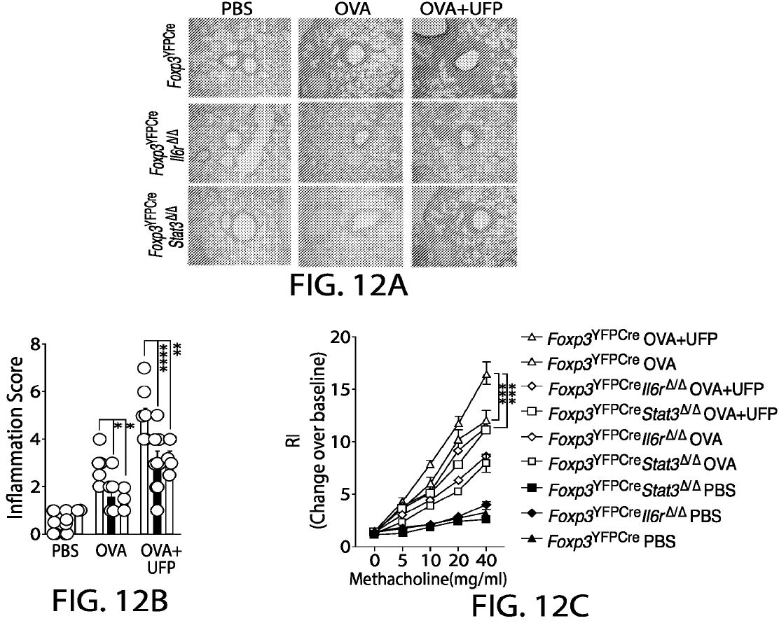

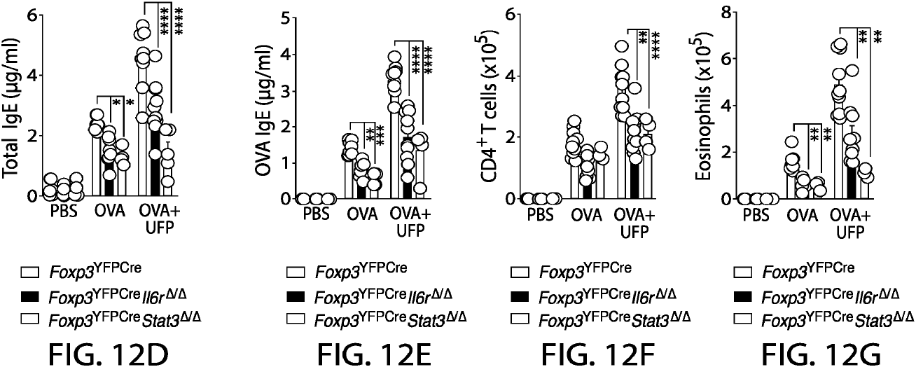

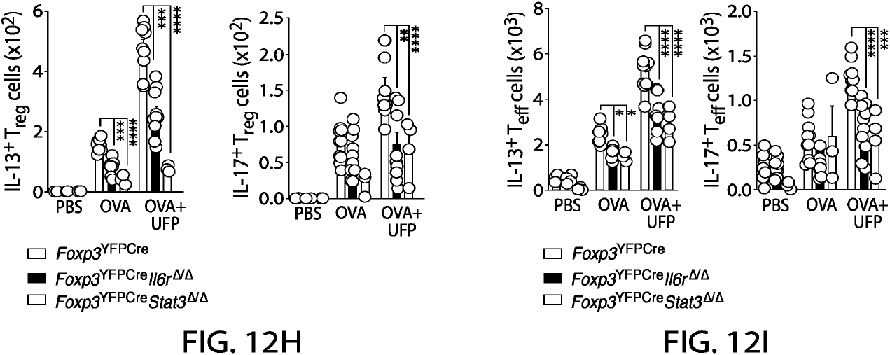

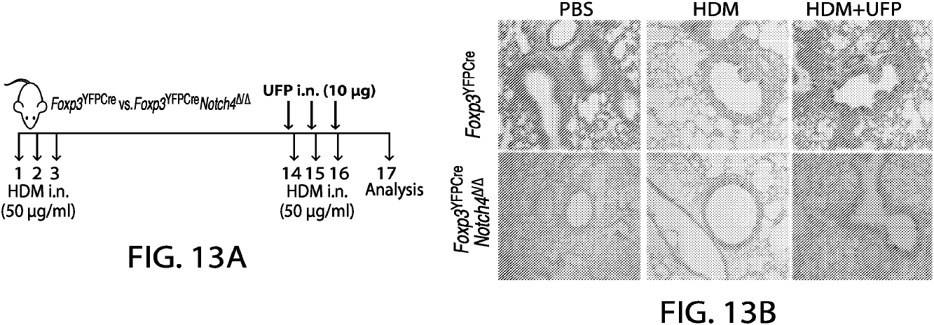

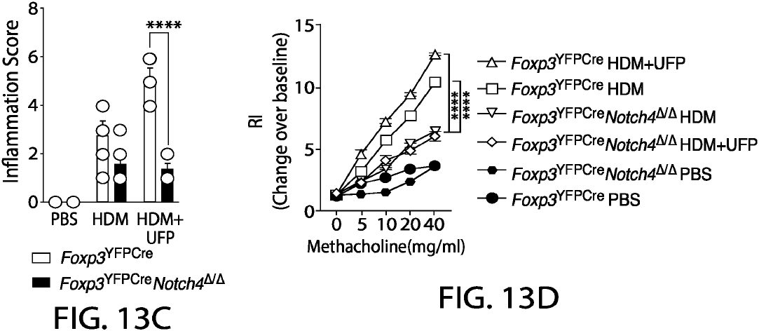

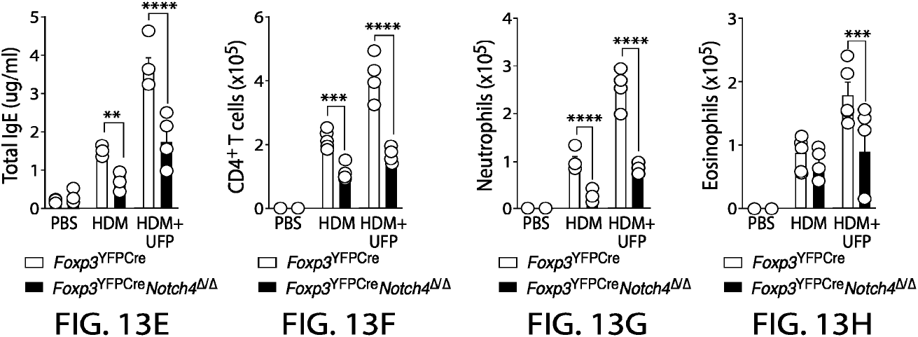

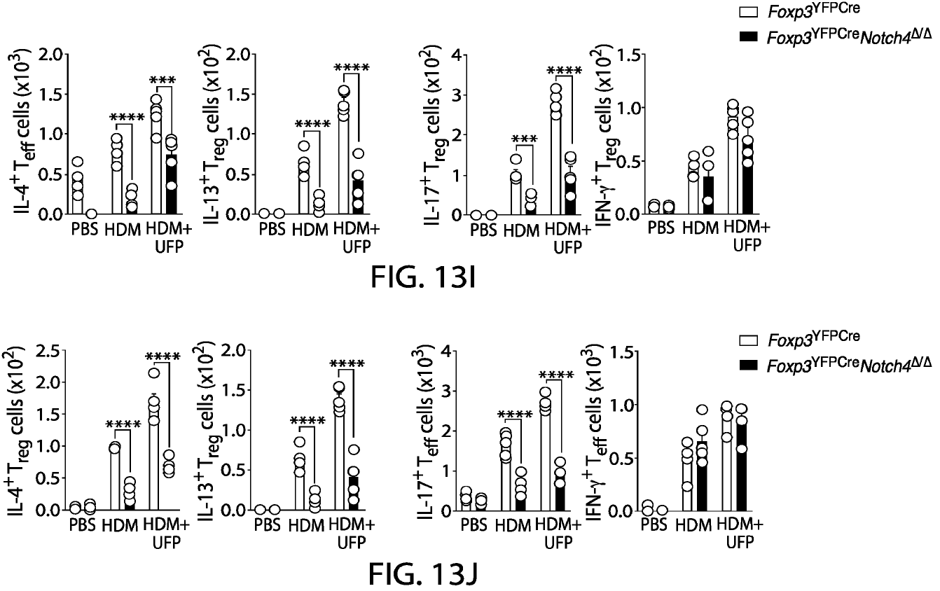

T_{reg} cell-specific Notch4 deletion was also found similarly protective in dust mite-induced allergic airway inflammation, where it also suppressed airway inflammation and hyper-responsiveness, tissue eosinophilia and neutrophilia and T_{H}2/T_{H}17 cell responses (FIGS. 12A-12I). It was also protective in a chronic model of allergic airway inflammation in which, in addition to suppressing the inflammatory and allergic responses noted above, it also suppressed sub-epithelial collagen deposition, a hallmark of airway remodeling due to chronic inflammation (FIGS. 13A-13K) (15).

### Notch4 Activates the Hippo and Wnt Pathways to Disrupt T_{reg} Cell Functions

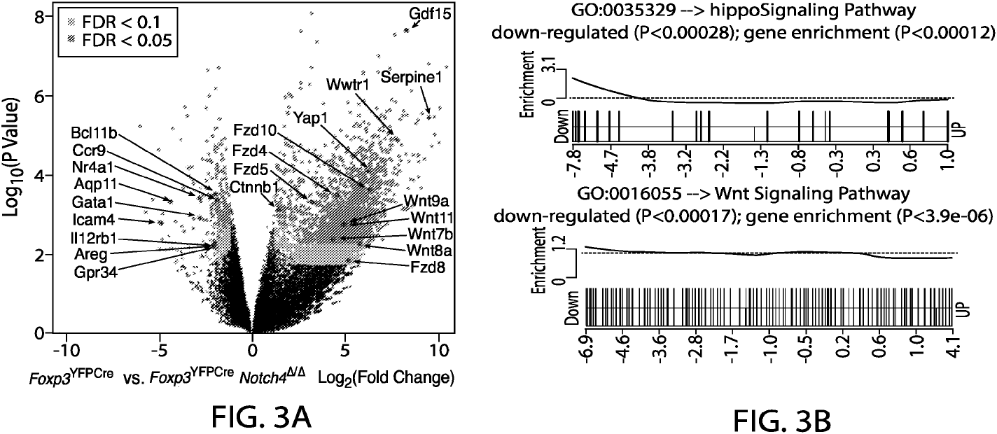

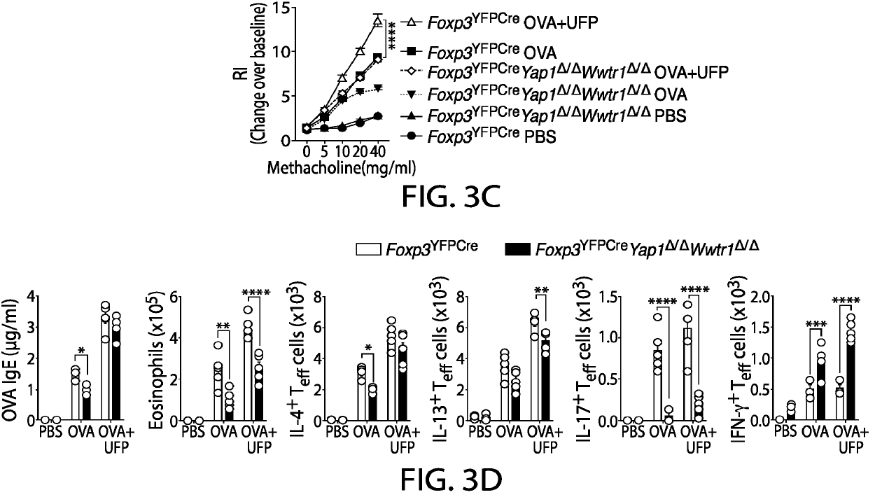

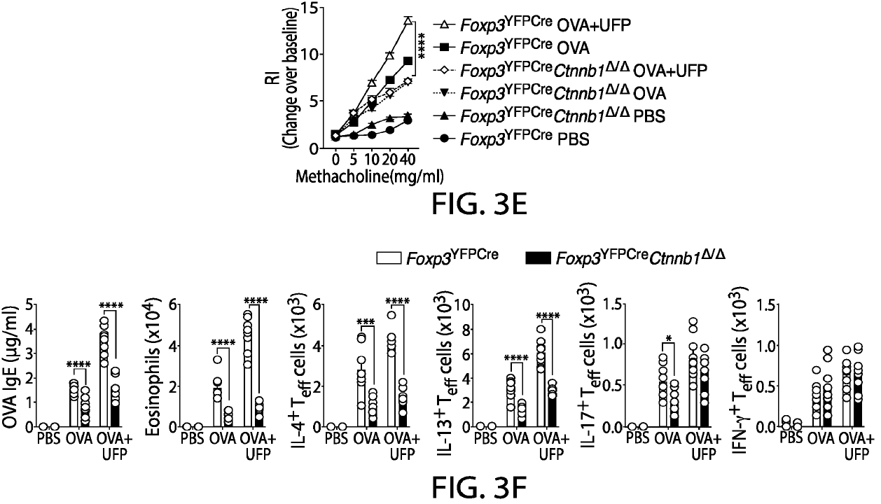

To further investigate the mechanisms by which Notch4 disrupted T_{reg} cell function, the inventors analyzed the transcriptional profiles of T_{reg} cells isolated from the lungs of sham and OVA+UFP treated Foxp3^{YFPCre} and Foxp3^{YFPCre}Notch4^{Δ/Δ} mice. Results revealed Notch4-dependent dysregulation of several pathways in OVA+UFP treated mice previously shown to impact T_{reg} cell stability and/or function, with particularly prominent changes in the hippo (Wwtrl, Yap1, Tead1/2/3/4, Foxo6) (16, 17), and Wnt pathways (Ctnnbl, Serpinel, Fzd5,8,10 and Wnt4,5a, 8a,9a, 9b,11) (18, 19) (FIGS. 3A and 3B; data not shown).

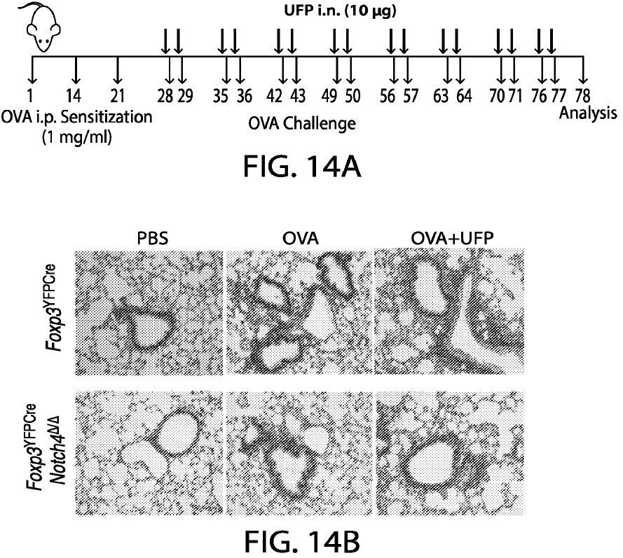

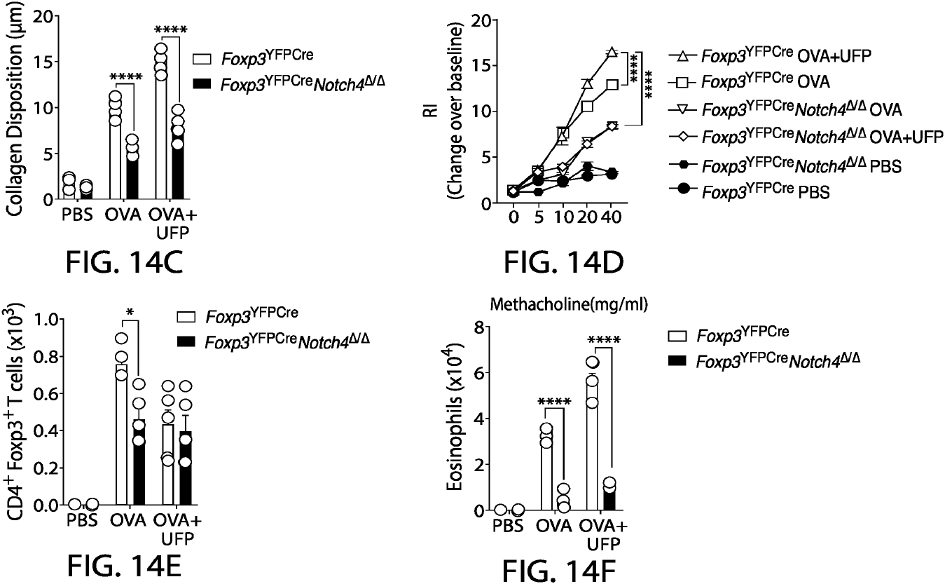

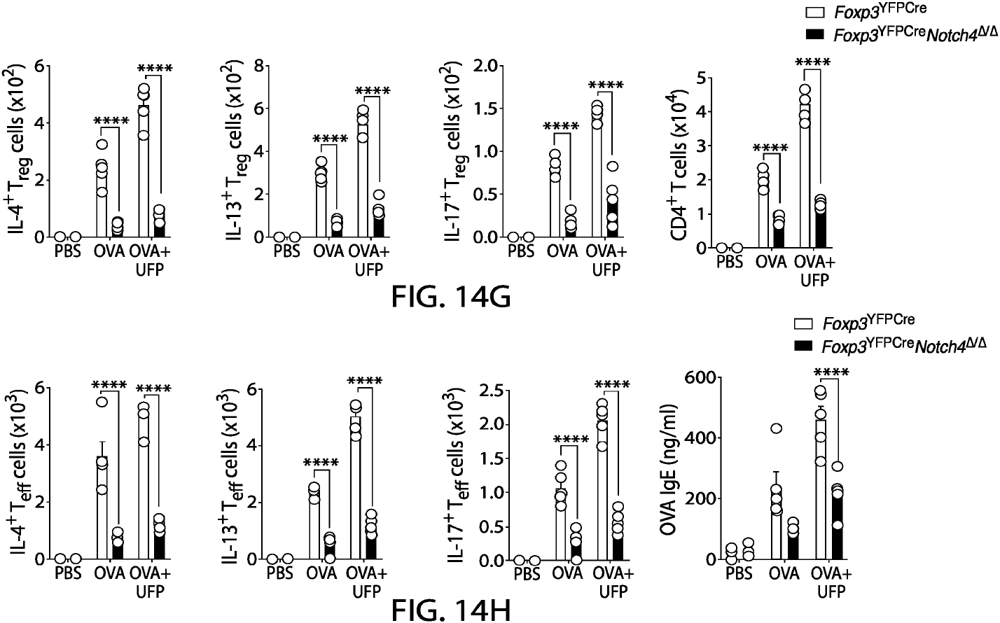

To determine the role of the Hippo and Wnt pathways in mediating T_{reg} cell subversion by Notch4, the inventors examined the consequences of T_{reg} cell-specific deletion of genes encoding key components of the respective pathways on allergic airway inflammation induced by OVA+UFP. Combined T_{reg} cell-specific deletion of Yap1 and Wwtrl, encoding the hippo pathway transcriptional regulators Yap and Taz (20), partially attenuated inflammation and AHR, whereas T_{reg} cell-specific deletion of Ctnnbl, encoding β-catenin (21), largely recapitulated the effect of T_{reg} cell-specific Notch4 deletion in suppressing those parameters, with neither deletions affecting Notch4 expression (FIGS. 3C and 3D; FIGS. 14A-14I). Yap1 and Wwtr1 deletion suppressed T_{H}17 and, to a lesser extent, T_{H}2 cell responses in the airways while upregulating T_{H}1 cell responses (FIG. 3E; FIGS. 14A-14I). In contrast, Ctnnbl deletion profoundly suppressed the T_{H}2 cell-like reprogramming of Notch4^{high} T_{reg} cells and the airway conventional T_{H}2 cell response but left the T_{H}17 cell responses unaffected (FIG. 3F; FIGS. 14A-14I). These results indicated that the Hippo and Wnt pathways mediated distinct, complimentary aspect of Notch4 signaling in disrupting immune tolerance in allergen and pollutant-induced allergic airway inflammation.

### Notch4 Promotes T_{reg} Cell Destabilization Towards T_{H}2 and T_{H}17 Cell Fates

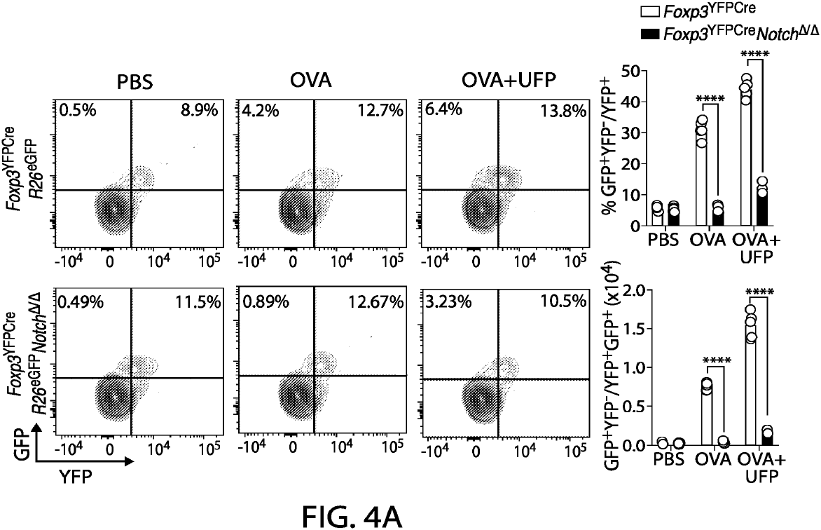

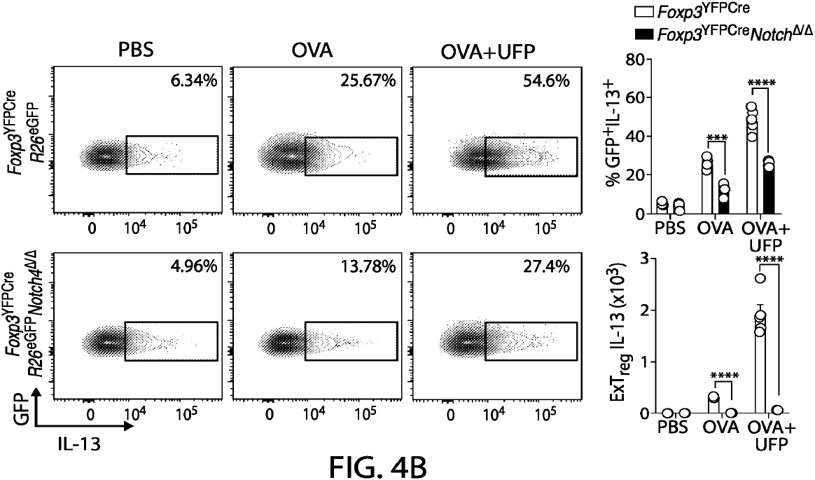

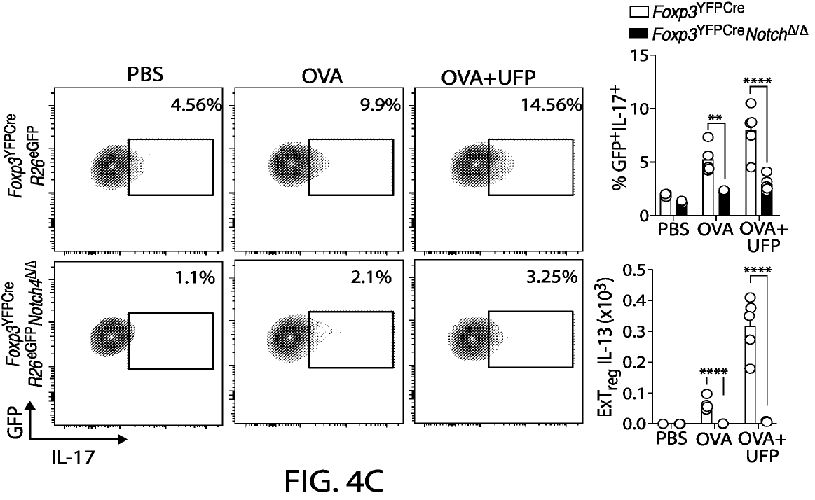

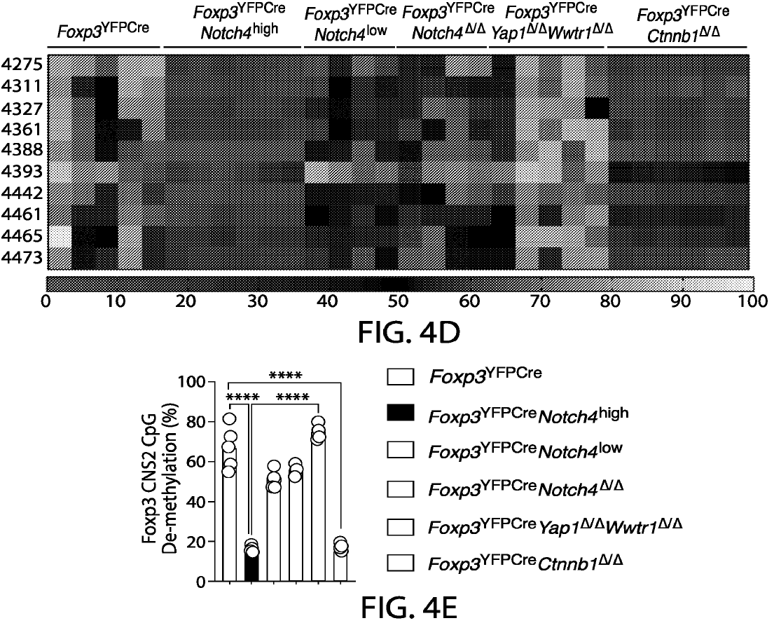

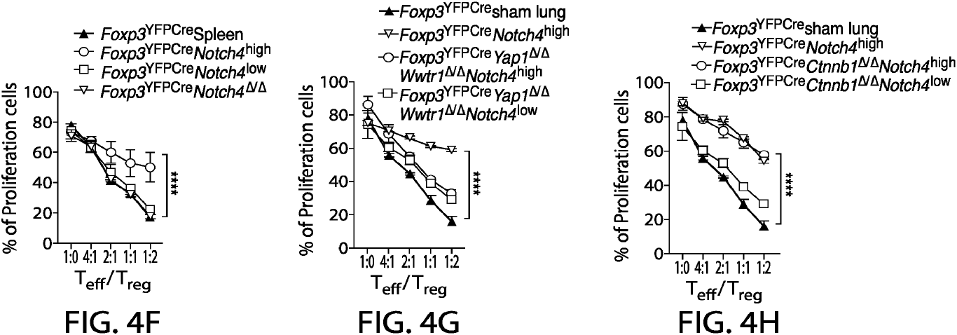

To determine whether Notch4 acted to destabilize lung T_{reg} cells in to give rise to Foxp3⁻ T_{H}2 and T_{H}17 ex-T_{reg} cells, the inventors employed a lineage tracing approach using a Rosa26 Stop-flox EGFP reporter (R26^{EGFP}) crossed to Foxp^{YFPCre}. Foxp3^{YFPCre}Notch4^{Δ/Δ}R26^{EGFP} and control Foxp3^{YFPCre}R26^{EGFP} mice were either sham or OVA sensitized than challenged with aerosolized OVA without or with intranasal UFP treatment. Cytokine expression was examined in T_{reg} (YFP⁺EGFP⁺), exT_{reg} (YFP⁻EGFP⁺) and CD4⁺ T_{eff} cells (YFP⁻EGFP⁻). It was found that the frequencies of YFP⁻EGFP⁺ exT_{reg} cells were markedly increased in the lungs of Foxp3^{YFPCre}R26^{EGFP} OVA sensitized and challenged group, and were further increased in the OVA+UFP treated group, with the exT_{reg} cells reaching up to a third of the total T_{reg} lineage-derived (EGFP⁺) cells in the lung (FIG. 4A). In contrast, the ex-T_{reg} cells were markedly decreased in the equivalent Foxp3^{YFPCre}Notch4^{Δ/Δ}R26^{EGFP} groups, indicative of heightened T_{reg} cell instability mediated by Notch4 (FIG. 4A). About half of the ex-T_{reg} cells were T_{H}2 and T_{H}17-skewed cells at a ratio of 3:1, with both being suppressed in Foxp3^{YFPCre}Notch4^{Δ/Δ}R26^{EGFP} mice (FIGS. 4B and 4C).

To investigate the source of Notch4-mediated T_{reg} cell instability, the inventors examined the epigenetic methylation signature of the Foxp3 CNS2 promoter region, which inversely affects T_{reg} cell lineage stability (22, 23). There was increased methylation of the Foxp3 CNS2 in T_{reg} cells isolated from the lungs of OVA+UFP-treated mice as compared to those of sham treated mice, which segregated with high but not low Notch4 expression (Notch4^{high} versus Notch4^{low}) (FIGS. 4D and 4E). Increased CNS methylation was also reversed upon T_{reg} cell-specific deletion of Notch4. Combined Yap1 and Wwtrl but not Ctnnbl deletion fully reversed the increased methylation of the Foxp3 CNS2 in lung T_{reg} cells of OVA/UFP-treated mice, indicating that the destabilization of T_{reg} cells by Notch4 signaling was mediated by the Hippo but not the Wnt pathway (FIGS. 4D and 4E).

The inventors furhter investigated the role of Notch4 expression in impairing T_{reg} cell function by sorting out Notch4^{high} versus Notch4^{low} T_{reg} cells from the lungs of Foxp3^{YFPCre} OVA+UFP treated group as well as T_{reg} cells from untreated control Foxp3^{YFPCre} mice and examining the three groups of T_{reg} cells for their suppressive capacity. Whereas the Notch4^{lo} lung T_{reg} cells from OVA+UFP treated mice were equivalent to control lung T_{reg} cells in their capacity to inhibit in vitro T cell proliferation, the suppressive function of Notch4^{high}T_{reg} cells was profoundly impaired (FIG. 4F). Combined Yap1 and Wwtrl but not Ctnnbl deletion fully restored the in vitro suppressive function of Notch4^{high} T_{reg} cells, consistent with the impact of the respective pathways on CNS2 demethylation (FIGS. 4G and 4H). Overall, these results indicated that Notch4 induced T_{reg} cell instability and T_{H}2/T_{H}17-cell-like programming in the context of allergic airway inflammation, and that this destabilization was associated with epigenetic methylation at the Foxp3 CNS2 locus mediated by the Hippo pathway.

### A T_{reg} Cell Notch4/Wnt/GDF15 Pathway Promotes ILC2 Expansion and Activation

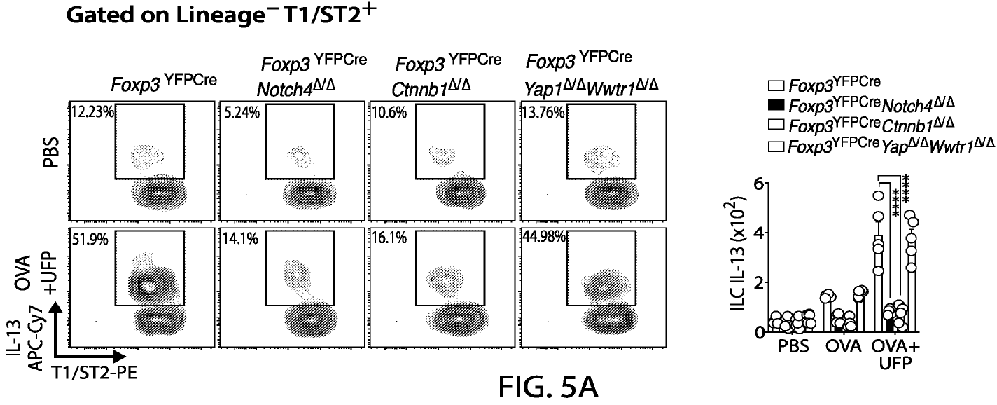

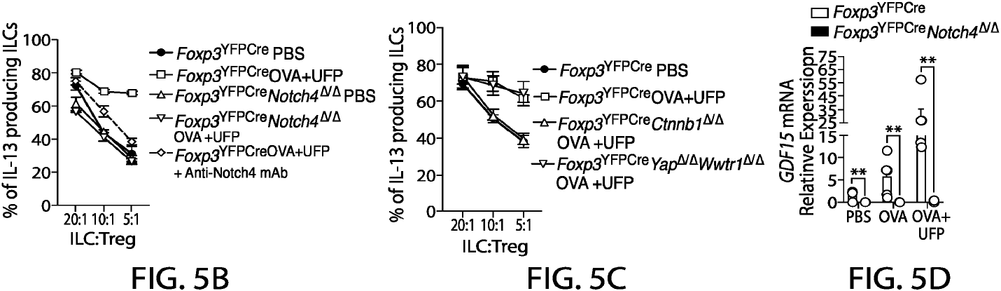

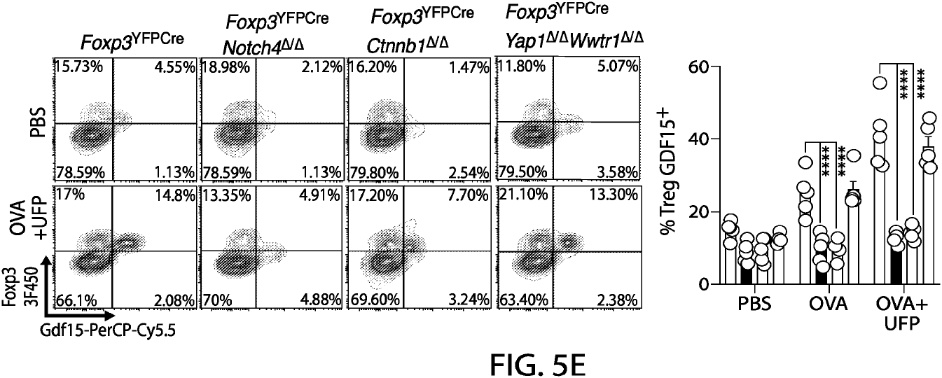

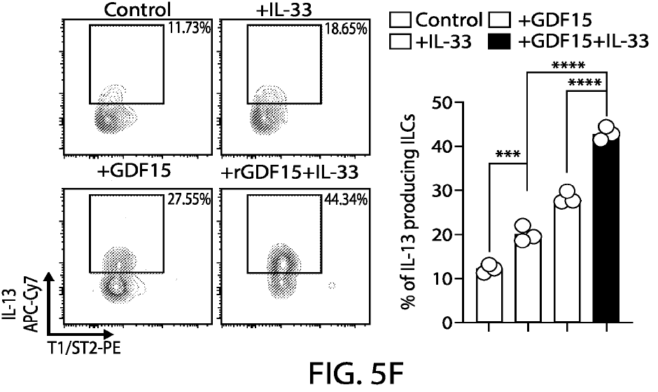

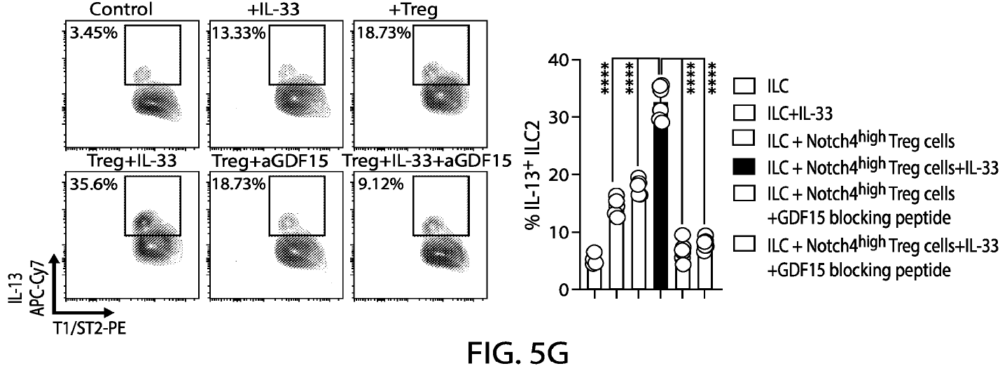

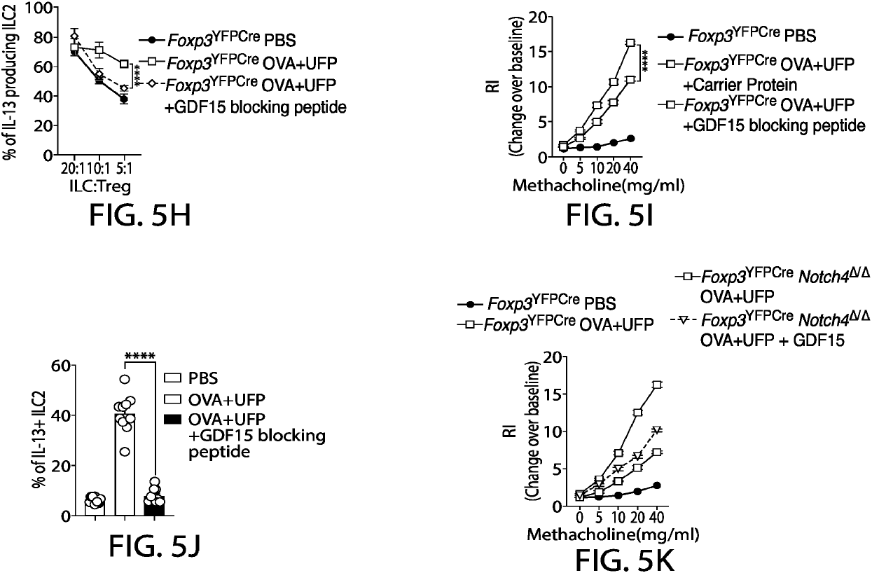

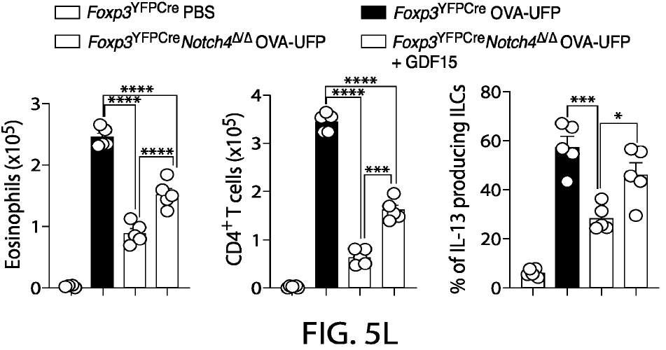

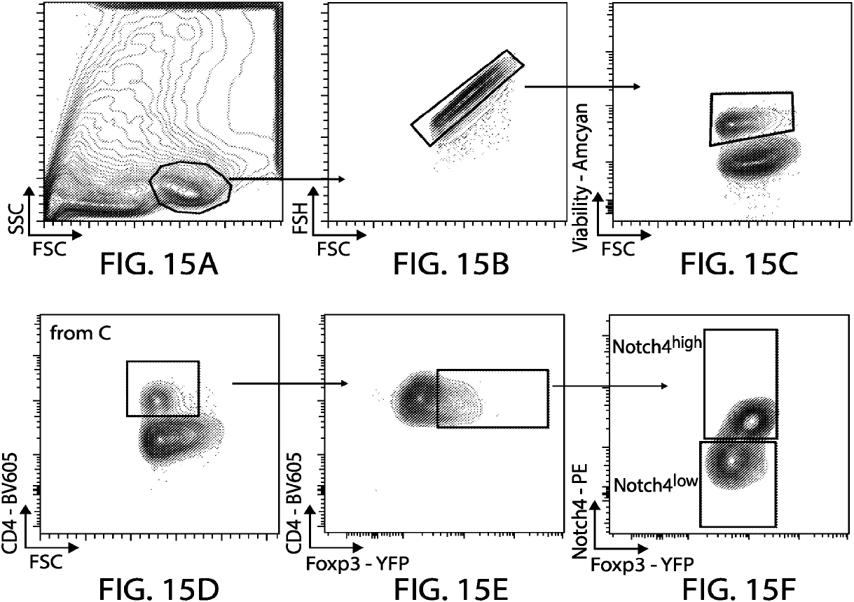

IL,C2 play a key role in allergic airway inflammation by virtue of copious secretion of T_{H}2 cytokines, most prominently IL-13 (24). Total ILC2 as well as IL-13-expressing ILC2 were sharply increased in OVA and especially OVA+UFP-treated mice but were dramatically reduced upon deletion of Notch4 in T_{reg} cells (FIG. 5A and FIGS. 15A-15F). The effect of T_{reg} cell-specific Notch4 deletion on ILC2 expansion and activation was reproduced by Ctnnbl but not Yap1/Wwtr1 deletion (FIG. 5A and FIGS. 15A-15F). In vitro studies revealed that Notch4^{high} T_{reg} cells derived from OVA+UFP-treated Foxp3^{YFPCre} mice failed to suppress the upregulation of IL13 expression in ILC2 derived from the inflamed lungs of the same mice (FIGS. 15A-15F). In contrast, Notch4^{low} lung T_{reg} cells derived from the OVA+UFP-treated Foxp3^{YFPCre} mice or Notch4-deficient T_{reg} cells derived from OVA+UFP-treated Foxp3^{YFPCre}Notch4^{Δ/Δ} mice potently suppressed IL-13 expression, as did treatment of Notch4^{high} T_{reg} cells with an anti-Notch4 mAb (FIG. 5B). Further analysis revealed that T_{reg} cell-specific Ctnnbl but not Yap/Taz deletion restored the ILC2 suppressive function of Notch4^{high} T_{reg} cells, thus implicating the Wnt pathway in the failure of IL,C2 regulation (FIG. 5C).

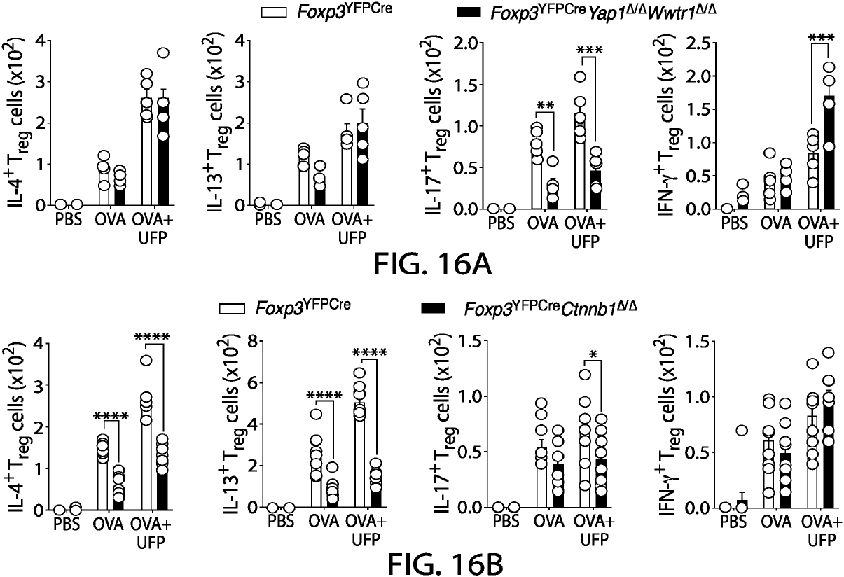

Transcripts encoding the cytokine GDF15 were highly induced in Notch4^{high} lung T_{reg} cell in a β-catenin-dependent manner (FIGS. 15A-15F). Flow cytometric analysis confirmed that T_{reg} cells were the primary source of GDF15 in the inflamed lungs of OVA and OVA+UFP treated mice, whereas it was sharply downregulated in Notch4- and β-catenin-deficient, but not Yap/Taz-deficient, T_{reg} cells (FIG. 5D; FIGS. 16A-16B). Addition of recombinant GDF15 to in vitro cultures of IL,C2 derived from naive mice upregulated IL-13 expression alone and especially in synergy with IL-33 (FIG. 5E). Furthermore, the in vitro co-culture of GDF15-expressing Notch4^{high} T_{reg} cells, isolated from lungs of OVA+UFP-treated mice, with naive ILC2 cells upregulated the expression of IL13 in the latter, an effect that was reversed by the addition of a GDF15 blocking peptide (FIG. 5F). Intra-tracheal Instillation of recombinant GDF15 upregulated the AHR and tissue inflammation in OVA+UFP treated Foxp3^{YFPCre}Notch4^{Δ/Δ} mice, as well as increasing IL-4 and IL-13 expression in T_{eff} cells (FIGS. 5G and H; FIGS. 15A-15F). Reciprocally, instillation of the GDF15 blocking peptide downregulated the aforementioned changes in OVA+UFP treated Foxp3^{YFPCre} mice (FIG. 5I and J; FIGS. 15A-15F). Overall, these findings confirmed that Notch4 expression abrogated the capacity of lung T_{reg} cells to suppress ILC2 and activated a T_{reg} cell-intrinsic Wnt-GDF15 axis that promoted ILC2 activation.

### T_{reg} Cell Notch4 Expression Segregates With Disease Severity in Asthmatics

To determine the relevance of Notch4 in asthma, the inventors analyzed the expression of Notch4 on peripheral blood mononuclear cells (PBMC) of asthmatic and control subjects.

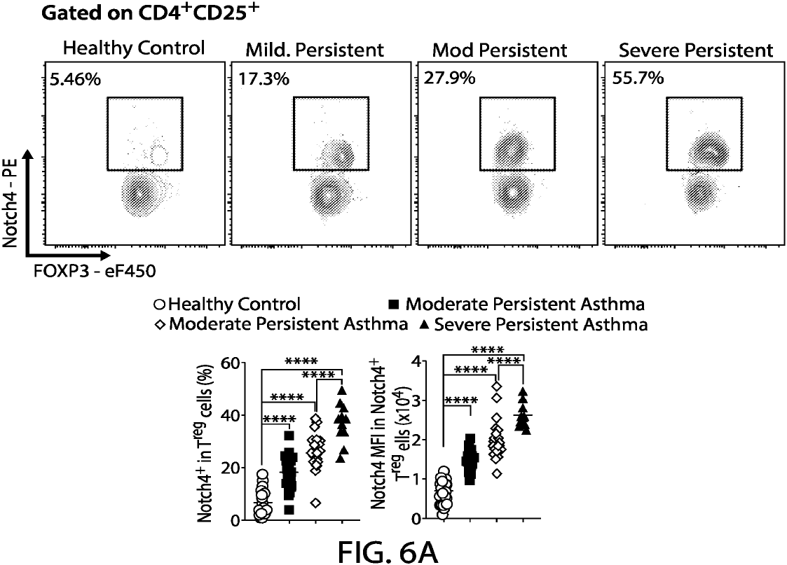

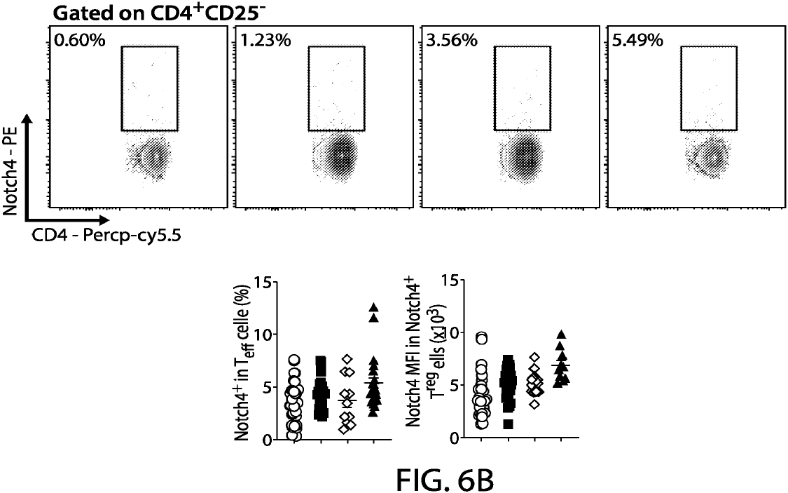

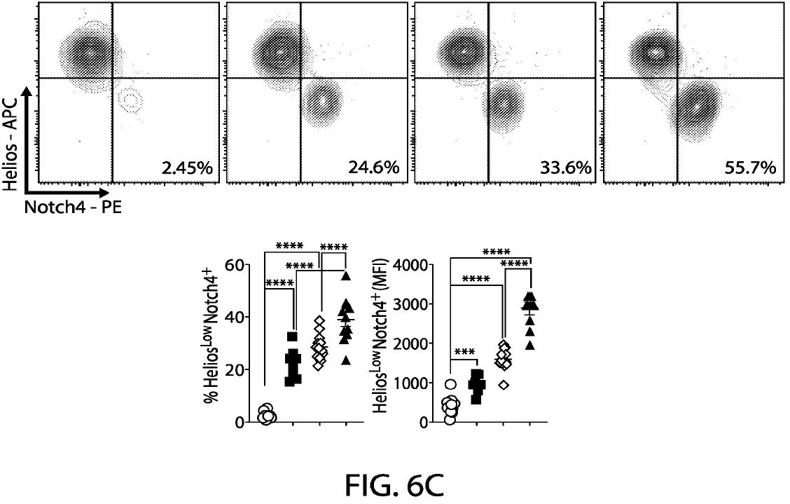

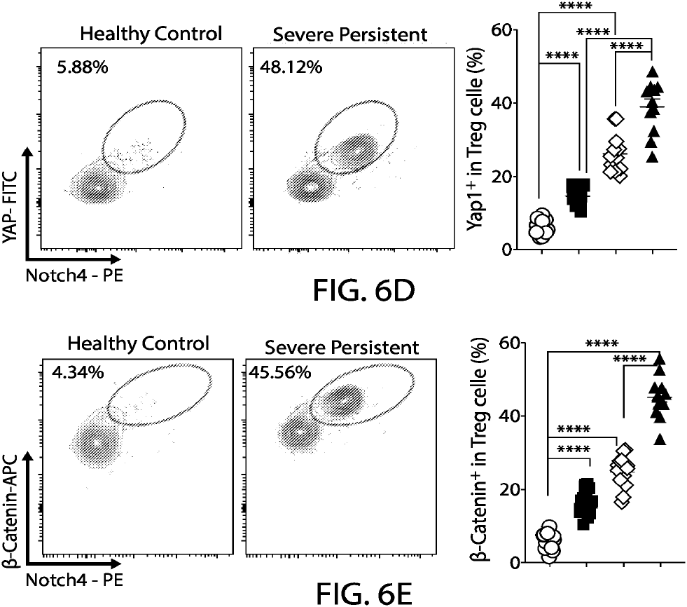

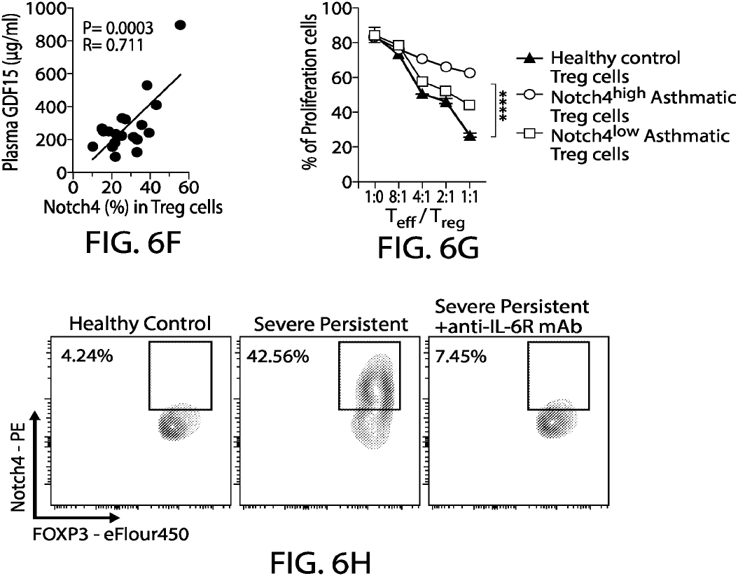

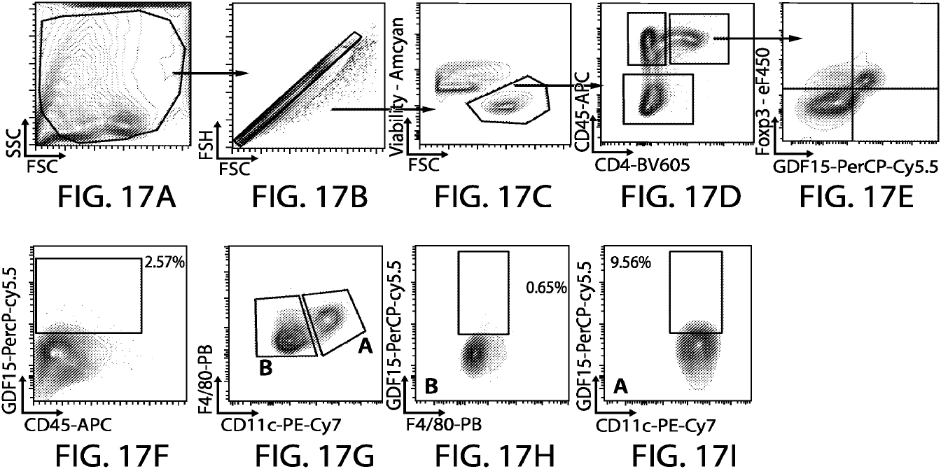

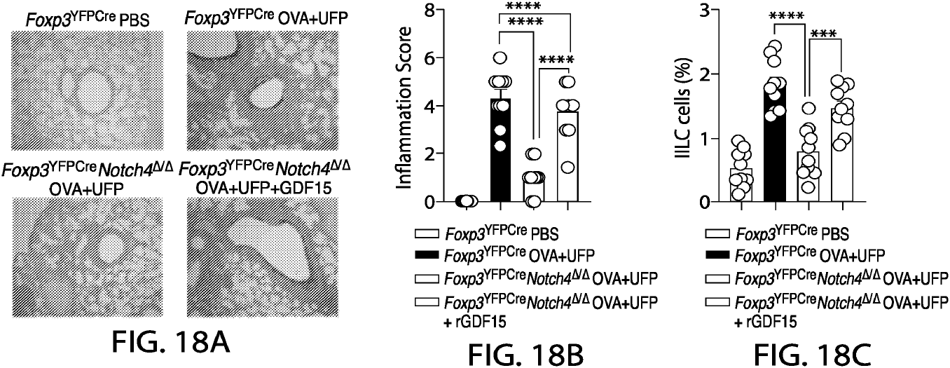

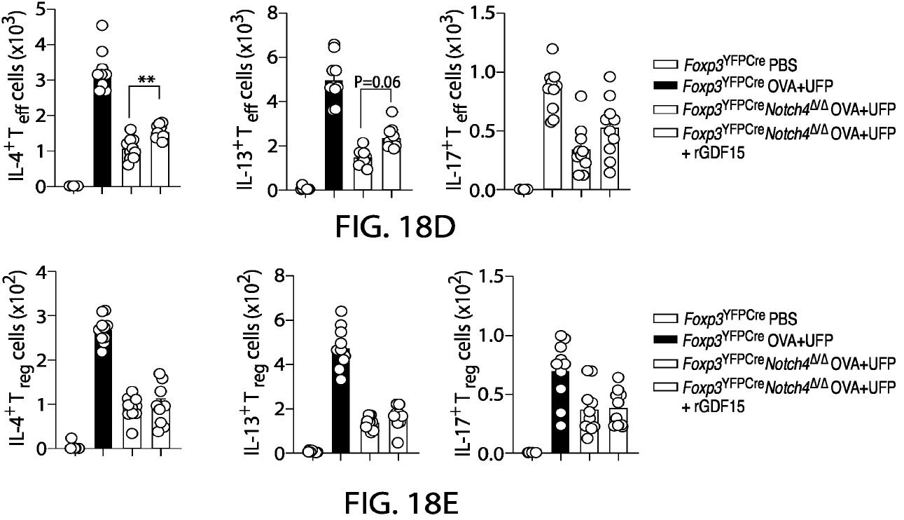

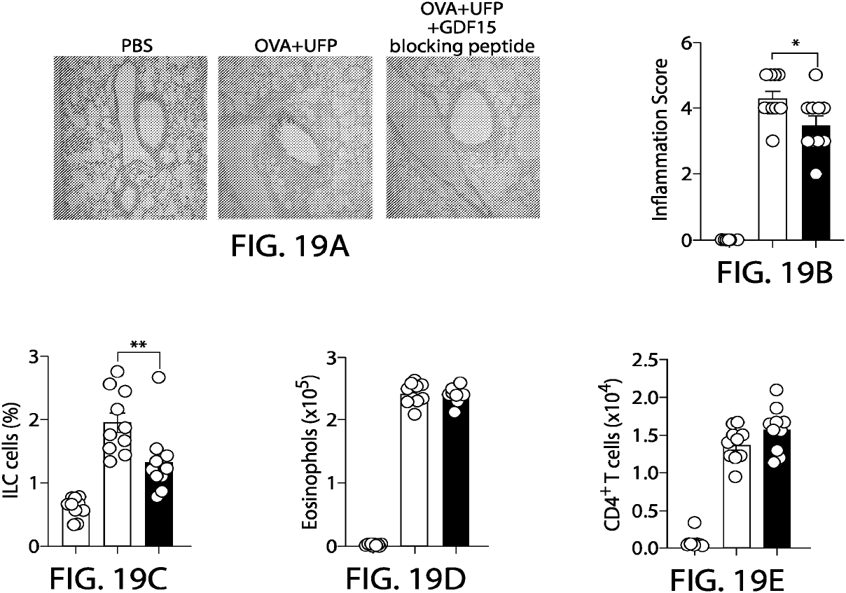

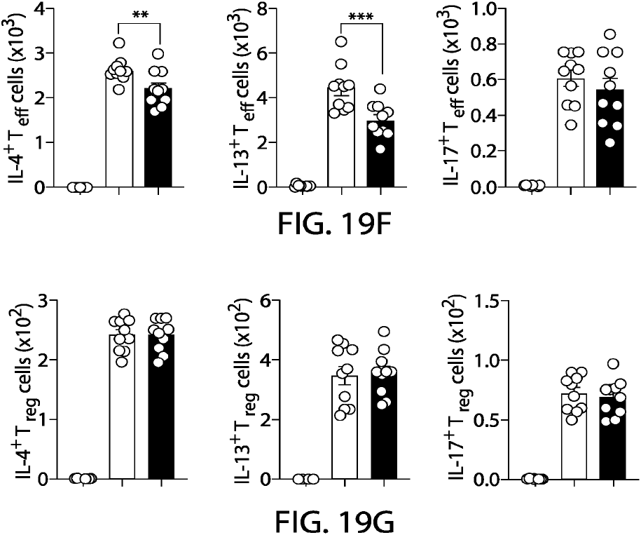

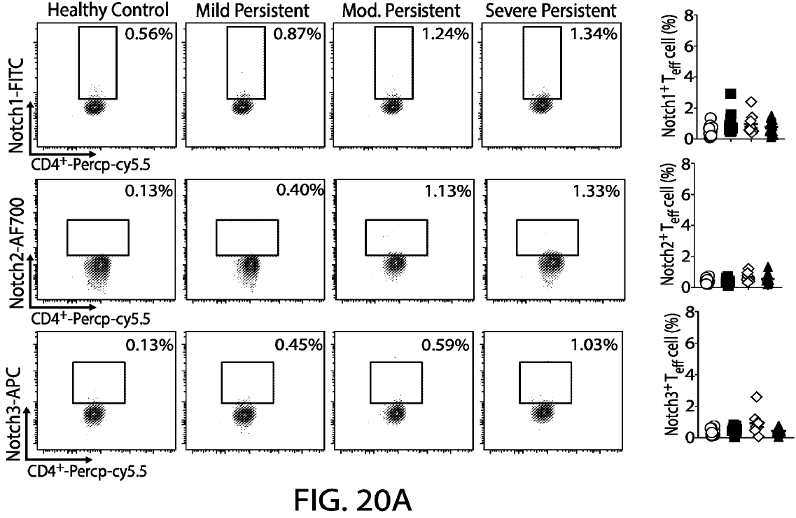

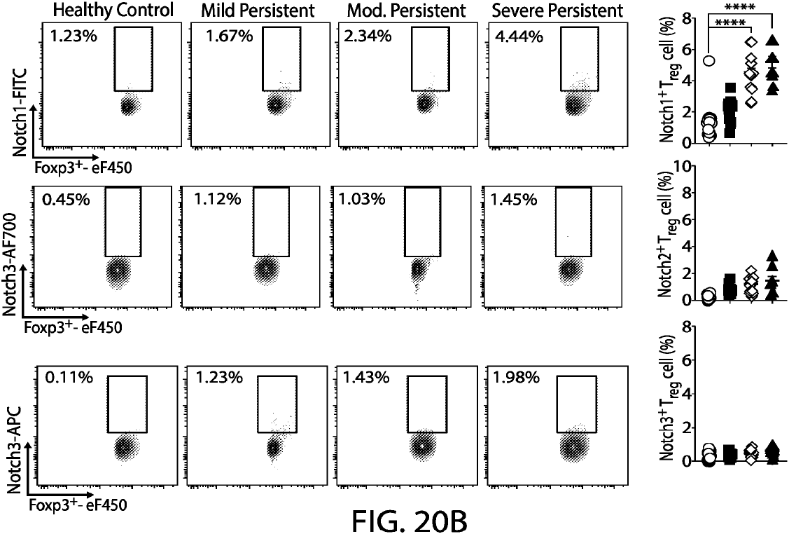

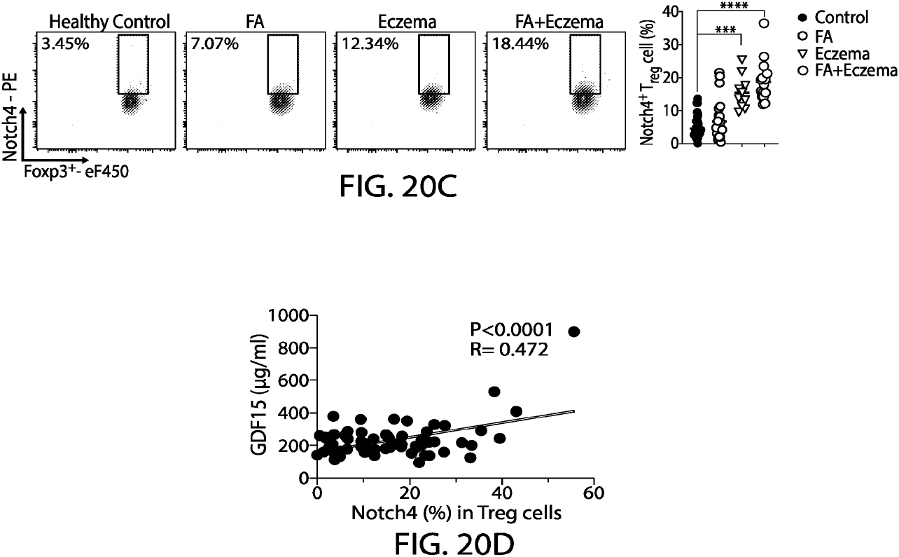

Results revealed that asthmatics had elevated frequencies of circulating Notch4⁺ T_{reg} cells, with both the cell frequencies and expression intensity progressively increasing as a function of asthma severity, reaching up to 50% of circulating T_{reg} cells in severe asthmatics (FIG. 6A). In contrast, Notch4 expression on circulating CD4⁺ T_{eff} cells was low and remained relatively flat as a function of asthma severity (FIG. 6B). Also, expression of Notchl-3 on T_{reg} and T_{eff} cells was not increased in asthmatics as compared to control subjects (FIGS. 17A-17I). Notch4 expression was restricted to the Helios^{low} iT_{reg} cell subpopulation (FIG. 6C). Further analysis revealed increased T_{reg} cell expression of the Hippo and Wnt pathway effector proteins Yap/Taz and β-catenin, respectively, which localized to Notch4⁺ T_{reg} cells and similarly increased as a function of asthma severity (FIGS. 6D and E). Also, there were increased concentrations of GDF 15 in the sera of moderate and severe asthmatics that positively correlated with circulating T_{reg} cell Notch4 expression (FIG. 6F). The contribution of Notch4 signaling to T_{reg} cell dysfunction was ascertained by the demonstration that Notch4^{high} peripheral blood T_{reg} cells poorly suppressed in vitro T cell proliferation as compared to Notch4^{low} T_{reg} cells isolated from the same asthmatic subjects or to T_{reg} cells isolated from healthy control subjects, which were overwhelmingly Notch4^{low} (FIG. 6G). Analysis of peripheral blood cells of a severe asthmatic subject treated with the anti-IL-6R mAb Tocilizumab revealed decreased Notch4 expression on the patient T_{reg} cells post therapy, consistent with the requirement for IL-6R signaling to upregulate Notch4 expression (FIG. 6H) (25). These results, which mirror those obtained in the mouse system, indicate that Notch4 expression may similarly serve as an immune regulatory switch that licenses allergic inflammation in human asthmatics.

### Discussion

A novel pathway central to the pathogenesis of asthmatic airway inflammation involving the inducible expression of Notch4 on allergen-specific T_{reg} cells is described in the data presented herein. This induction, synergistically mediated by allergens and ambient pollutant particles, activates downstream Hippo and Wnt pathways to subvert T_{reg} cell stability and functions. Inhibition of Notch4 expression in T_{reg} cells, but not that of other Notch receptors, suppressed airway inflammation and restored immune tolerance. Critically, Notch4 signaling upregulated the expression in T_{reg} cells of GDF 15, a cytokine that the inventors demonstrate to reinforce airway inflammation by a novel mechanism involving ILC2 activation. Notch4 and its downstream effector pathways were upregulated on T_{reg} cells of asthmatic subjects as a function of disease severity, thus identifying Notch4 as an immune regulatory switch mechanism that licenses asthmatic inflammation and highlighting the therapeutic potential for tolerance restoration in asthma.

Induction of Notch4 on T_{reg} cells in the airway involved coordinate allergen peptide-specific TCR activation and IL-6/STAT3 signaling, a process further upregulated by IL-33. Remarkably, this pathway thus integrates several genetic loci, including NOTCH4, IL6 and IL33, identified to impart susceptibility to asthma incidence and/or disease severity (26-30). T_{reg} cell-specific deletion of Il6ra or Stat3 substantially attenuated Notch4 expression in T_{reg} cells in allergic airway inflammation, as did treatment of a severe asthmatic subject with the anti-IL-6Ra chain mAb tocilizumab (25). STAT3 was demonstrated to bind to the Notch4 promoter, consistent with direct upregulation of Notch4 expression by IL,-6/STAT3 signaling. The specificity of Notch4 induction on lung T_{reg} cells may relate to a “niche effect”, in which the interaction with alveolar macrophages normally drives differentiation of naive allergen specific T cells into T_{reg} cells (8, 31). The uptake of allergens and ambient particulate matter is associated with the production of IL-6 and upregulation of Notch ligands including Jagged 1 (8), driving T_{reg} skewing towards T_{eff} cell phenotypes in a Notch4-dependent manner.

Notch4 expression on lung T_{reg} cells mobilized several downstream pathways, notably Hippo and Wnt, to derail T_{reg} cell stability and function. Expression of effectors of both pathways, including Yap and Taz (Hippo) and β-catenin (Wnt) also segregated with Notch4 expression in peripheral blood T_{reg} cells of human asthmatics and correlated with asthma severity. The two pathways acted to disrupt different aspects of T_{reg} cell functions. Whereas the Hippo pathway impaired lung T_{reg} cell in vitro suppressor function and promoted their skewing towards the Th17 cell lineage, the Wnt pathway promoted their Th2 cell-like reprogramming and was essential to the Th2 effector T cell response in the airways. Thus, Notch4 mobilizes distinct signaling pathways within lung T_{reg} cells that act in a modular fashion to disrupt immune tolerance in the airways.

An important pathogenic mechanism mobilized by Notch4 is the potentiation of ILC2 activation, which proceeded by a T_{reg} cell-intrinsic, beta catenin-dependent pathway. Notch4-beta catenin signaling impaired the suppression by T_{reg} cells of activated ILC2. Furthermore, it positively promoted the activation of resting ILC2 by inducing the expression in T_{reg} cells of GDF15, which activated ILC2 in synergy with IL-33. Antagonism of GDF15 augmented the in vitro suppression by Notch4^{high} T_{reg} cells of ILC2 activation and downregulated airway inflammation in vivo. These results indicated a critical role for GDF15 in mediating an ILC2-dependent forward amplificatory loop by which Notch4^{high} T_{reg} cells actively promote asthmatic inflammation.

Analysis of Notch4 expression on circulating T_{reg} cells of a pediatric cohort of asthmatic subjects demonstrated a step-wise increase in Notch4 expression as a function of asthma severity, with the T_{reg} cells of severe asthmatics especially marked by high expression of Notch4 and its downstream effectors Yap/Taz and beta catenin. Similar to the mouse studies, Notch4-expressing human T_{reg} cells showed impaired in vitro suppressive function. Remarkably, there was minimal heterogeneity in Notch4 expression within each disease severity subgroup, highlighting Notch4 as a common pathogenic mechanism operative in these patients whose amplitude is highly informative of disease activity. These results emphasize the usefulness of Notch4 and its down-stream Hippo/Wnt effectors as novel biomarkers to monitor disease activity and response to therapy, and therapeutic targets for treating and/or preventing the disease.

In conclusion, identified herein is a novel mechanism central to the pathogenesis of asthmatic inflammation.

### References

1. B. N. Lambrecht, H. Hammad, The immunology of asthma. Nat Immunol 16, 45-56 (2015).
2. F. D. Martinez, D. Vercelli, Asthma. Lancet 382, 1360-1372 (2013).
3. M. Noval Rivas, T. A. Chatila, Regulatory T cells in allergic diseases. J Allergy Clin Immunol 138, 639-652 (2016).
4. N. Krishnamoorthy et al., Early infection with respiratory syncytial virus impairs regulatory T cell function and increases susceptibility to allergic asthma. Nat Med 18, 1525-1530 (2012).
5. M. Noval Rivas et al., Regulatory T cell reprogramming toward a Th2-cell-like lineage impairs oral tolerance and promotes food allergy. Immunity 42, 512-523 (2015).
6. A. H. Massoud et al., An asthma-associated IL,4R variant exacerbates airway inflammation by promoting conversion of regulatory T cells to TH17-like cells. Nat Med 22, 1013-1022 (2016).
7. M. Xia et al., Vehicular exhaust particles promote allergic airway inflammation through an aryl hydrocarbon receptor-notch signaling cascade. J Allergy Clin Immunol 136, 441-453 (2015).
8. M. Xia, H. Harb, A. Saffari, C. Sioutas, T. A. Chatila, A Jagged 1-Notch 4 molecular switch mediates airway inflammation induced by ultrafine particles. J Allergy Clin Immunol142, 1243-1256 e1217 (2018).
9. V. W. W. Tsai, Y. Husaini, A. Sainsbury, D. A. Brown, S. N. Breit, The MIC-⅟GDF15-GFRAL Pathway in Energy Homeostasis: Implications for Obesity, Cachexia, and Other Associated Diseases. Cell Metab 28, 353-368 (2018).
10. H. H. Luan et al., GDF15 Is an Inflammation-Induced Central Mediator of Tissue Tolerance. Cell 178, 1231-1244 e1211 (2019).
11. W. Chen et al., Conversion of peripheral CD4+CD25- naive T cells to CD4+CD25+ regulatory T cells by TGF-beta induction of transcription factor Foxp3. J Exp Med 198, 1875-1886 (2003).
12. S. Shi, P. Stanley, Protein O-fucosyltransferase 1 is an essential component of Notch signaling pathways. Proc Natl Acad Sci USA 100, 5234-5239 (2003).
13. L. M. Charbonnier, S. Wang, P. Georgiev, E. Sefik, T. A. Chatila, Control of peripheral tolerance by regulatory T cell-intrinsic Notch signaling. Nat Immunol 16, 1162-1173 (2015).
14. H. Han et al., Inducible gene knockout of transcription factor recombination signal binding protein-J reveals its essential role in T versus B lineage decision. Int Immunol 14, 637-645 (2002).
15. R. Tachdjian et al., Pathogenicity of a disease-associated human IL-4 receptor allele in experimental asthma. J Exp Med 206, 2191-2204 (2009).
16. H. Shi et al., Hippo Kinases Mst1 and Mst2 Sense and Amplify IL-2R-STAT5 Signaling in Regulatory T Cells to Establish Stable Regulatory Activity. Immunity 49, 899-914 e896 (2018).
17. J. Geng et al., The transcriptional coactivator TAZ regulates reciprocal differentiation of TH17 cells and Treg cells. Nat Immunol 18, 800-812 (2017).
18. J. van Loosdregt et al., Canonical Wnt signaling negatively modulates regulatory T cell function. Immunity 39, 298-310 (2013).
19. J. van Loosdregt, P. J. Coffer, The Role of WNT Signaling in Mature T Cells: T Cell Factor Is Coming Home. J Immunol 201, 2193-2200 (2018).
20. J. R. Misra, K. D. Irvine, The Hippo Signaling Network and Its Biological Functions. Annu Rev Genet 52, 65-87 (2018).
21. H. Clevers, R. Nusse, Wnt/beta-catenin signaling and disease. Cell 149, 1192-1205 (2012).
22. Y. Feng et al., Control of the inheritance of regulatory T cell identity by a cis element in the Foxp3 locus. Cell 158, 749-763 (2014).
23. X. Li, Y. Liang, M. LeBlanc, C. Benner, Y. Zheng, Function of a Foxp3 cis-element in protecting regulatory T cell identity. Cell 158, 734-748 (2014).
24. E. Vivier et al., Innate Lymphoid Cells: 10 Years On. Cell 174, 1054-1066 (2018).
25. B. Esty et al., Treatment of severe persistent asthma with IL-6 receptor blockade. J Allergy Clin Immunol Pract 7, 1639-1642 e1634 (2019).
26. T. Hirota et al., Genome-wide association study identifies three new susceptibility loci for adult asthma in the Japanese population. Nat Genet 43, 893-896 (2011).
27. M. A. Ferreira et al., Identification of IL6R and chromosome 11q13.5 as risk loci for asthma. Lancet 378, 1006-1014 (2011).
28. O. Gruzieva et al., Prenatal Particulate Air Pollution and DNA Methylation in Newborns: An Epigenome-Wide Meta-Analysis. Environ Health Perspect 127, 57012 (2019).
29. X. Li et al., Genome-wide association study identifies TH1 pathway genes associated with lung function in asthmatic patients. J Allergy Clin Immunol 132, 313-320 e315 (2013).
30. O. E. Savenije et al., Association of IL33-IL-1 receptor-like 1 (IL1RL1) pathway polymorphisms with wheezing phenotypes and asthma in childhood. J Allergy Clin Immunol 134, 170-177 (2014).
31. P. Soroosh et al., Lung-resident tissue macrophages generate Foxp3+ regulatory T cells and promote airway tolerance. J Exp Med 210, 775-788 (2013).
32. C. N. Magee et al., Notch-1 Inhibition Promotes Immune Regulation in Transplantation via Treg-Dependent Mechanisms. Circulation, (2019).

1. A method for treating asthma or an allergic disease, comprising administering to a subject having asthma or an allergic disease an effective amount of an agent that inhibits Wnt signaling.

2. A method for treating asthma or an allergic disease, comprising administering to a subject having asthma or an allergic disease an effective amount of an agent that inhibits Hippo signaling.

3. A method for treating asthma or an allergic disease, comprising administering to a subject having asthma or an allergic disease an effective amount of an inhibitor of Growth-differentiation factor 15 (GDF15).

4. The method of claims 1–2, further comprising administering an agent that inhibits Growth/differentiation factor 15 (GDF15).

5. The method of any of claims 1–3, further comprising administering an agent that inhibits Notch4.

6. The method of claim 3, further comprising administering an agent that inhibits Wnt signaling and an agent that inhibits Hippo signaling.

7. The method of any of claims 1–6, further comprising, prior to administering, diagnosing a subject as having asthma or an allergic disease.

8. The method of any of claims 1–6, further comprising, prior to administering, receiving the results of an assay that diagnoses a subject as having asthma or an allergic disease.

9. The methods of any of claims 1–8, wherein the asthma is selected from the list consisting of allergic asthma, asthma without allergies, aspirin exacerbated respiratory disease, exercise induced asthma, cough variant, and occupational asthma.

10. The methods of any of claims 1–8, wherein the allergic disease is selected from the list consisting of allergic rhinitis, sinusitis, otitis media, atopic dermatitis, urticaria, angioedema, and anaphylaxis.

11. The method of any of claims 1–6, wherein the agent is selected from the group consisting of a small molecule, an antibody, a peptide, a genome editing system, an antisense oligonucleotide, and an RNAi.

12. The method of claim 11, wherein the antibody is a humanized antibody.

13. The method of claim 11, wherein the RNAi is a microRNA, an siRNA, or a shRNA.

14. The method of claim 11, wherein the small molecule is an inhibitor of Wnt signaling, and is selected from the group consisting of XAV-939, ICG-001, IWR-1-endo, Wnt-C59 (C59), LGK-974, JW55, ETC-159, iCRT14, KY02111, IWP-2, IWP-L6, Isoquercitrin, PNU-74654, CP21R7 (CP21), Salinomycin (from Streptomyces albus), Adavivint (SM04690), FH535, IWP-O1, LF3, WIKI4, Triptonide, PRI-724, GNF-6231, KYA1797K, Methyl Vanillate, iCRT3, WAY-316606, and SKL2001.

15. The method of claim 11, wherein the small molecule is an inhibitor of Hippo signaling, and is selected from the group consisting of (R)-PFI 2 hydrochloride, Verteporfin, YAP inhibitor, XMU MP 1, Ki 16425, and Ro 08-2750.

16. The method of claim 11, wherein the peptide is an inhibitor of GDF 15, and has a sequence of (aa258-273).

17. The method of claims 11–12, wherein the antibody is an anti-Notch4 antibody.

18. The method of claim 3, wherein inhibiting GDF15 is inhibiting GFD 15 expression level or activity.

19. The method of claim 1, 6, and 14, wherein inhibiting Wnt signaling reduces the population of Th2 effector cells.

20. The method of claim 2, 6, and 15, wherein inhibiting Hippo signaling reduces the population of Th17 effector cells.

21. The method of claim 3, 6, and 15, wherein inhibiting GDF15 reduces the population of group 2 innate lymphoid cell (ILC2).

22. The method of any of claims 19–21, wherein the population is reduced at least 50%, 60%, 70%, 80%, 90%, 95%, or more are compared to an appropriate control.

23. The method of any of claims 1–22, further comprising administering at least one additional anti-asthma therapeutic.

24. The method of any of claims 1–22, further comprising administering at least one additional anti-allergic disease therapeutic.

25. A method for preventing asthma or an allergic disease, comprising administering to a subject at risk of having asthma or an allergic disease an effective amount of an agent that inhibits Wnt signaling.

26. A method for preventing asthma or an allergic disease, comprising administering to a subject at risk of having asthma or an allergic disease an effective amount of an agent that inhibits Hippo signaling.

27. A method for preventing asthma or an allergic disease, comprising administering to a subject at risk of having asthma or an allergic disease an effective amount of an inhibitor of Growth-differentiation factor 15 (GDF15).

28. The method of claims 25–27, further comprising administering an agent that inhibits GDF15.

29. The method of any of claims 25–28, further comprising administering an agent that inhibits Notch4.

30. The method of claim 27, further comprising administering an agent that inhibits Wnt signaling and an agent that inhibits Hippo signaling.

31. The method of any of claims 25–30, further comprising, prior to administering, diagnosing a subject as being at risk of having asthma or an allergic disease.

32. The method of any of claims 25–30, further comprising, prior to administering, receiving the results of an assay that diagnoses a subject as being at risk of having asthma or an allergic disease.

33. A composition for preventing or treating asthma or an allergic disease, comprising an agent that inhibits Wnt signaling and a pharmaceutically acceptable carrier.

34. A composition for preventing or treating asthma or an allergic disease, comprising an agent that inhibits Hippo signaling and a pharmaceutically acceptable carrier.

35. A composition for preventing or treating asthma or an allergic disease, comprising an agent that inhibits GDF15 and a pharmaceutically acceptable carrier.

36. The composition of claims 33–34, further comprising an agent that inhibits GDF 15.

37. The composition of any of claims 33–36, further comprising an agent that inhibits Notch4.

38. The composition of claim 35, further comprising an agent that inhibits Wnt signaling and an agent that inhibits Hippo signaling.

39. An agent that inhibits the Wnt signaling pathway.

40. An agent that inhibits the Hippo signaling pathway.

41. An agent that inhibits GDF 15.

42. An agent that inhibits Notch4.

43. A method for treating asthma or an allergic disease, the method comprising:
d. obtaining a biological sample from a subject;
e. measuring the level of Notch4 in the biological sample of (a);
f. comparing the level of (b) with a reference level, wherein a subject is identified as having asthma or an allergic disease if the level of (b) is greater than a reference level; and
d. administering to the subject identified as having at risk asthma or an allergic disease any of the compositions of claims 33–38, or agents of claims 39–42.

44. A method for preventing asthma or an allergic disease, the method comprising:
d. obtaining a biological sample from a subject;
e. measuring the level of Notch4 in the biological sample of (a);
f. comparing the level of (b) with a reference level, wherein a subject is identified as being at risk of having asthma or an allergic disease if the level of (b) is greater than a reference level; and
d. administering to the subject identified as having at risk asthma or an allergic disease any of the compositions of claims 33–38, or agents of claims 39–42.
